(19) United States
(12) Patent Application Publication
(10) Pub. No.: US 2023/0279185 A1
(43) Pub. Date: Sep. 7, 2023

(54) ENGINEERED COMPOSITION OF PHENOLIC COMPOUNDS

(71) Applicant: KATHOLIEKE UNIVERSITEIT LEUVEN, Leuven (BE)

(72) Inventors: Bert LAGRAIN, Kessel-Lo (BE); Bert SELS, Westerlo (BE); Joost VAN AELST, Hasselt (BE); Korneel VAN AELST, Antwerpen (BE); Sander VAN DEN BOSCH, Boortmeerbeek (BE)

(21) Appl. No.: 18/018,120

(22) PCT Filed: Jul. 29, 2021
(86) PCT No.: PCT/EP2021/071357

§ 371 Date: Jan. 26, 2023

(30) Foreign Application Priority Data
Jul. 29, 2020 (GB) 2011741.2

(51) Int. Cl. C08H 7/00; C08J 3/11; C08J 11/10

(52) U.S. Cl. CPC C08H 6/00; C08J 3/11; C08J 11/105; C08J 2397/02

(57) ABSTRACT

A complex non-naturally occurring phenolic compounds mixtures or engineered phenolic compounds compositions, from catalytic degradation of lignocellulose, and the use thereof.

### FIELD OF THE INVENTION

The present invention relates to non-naturally occurring phenolic compound compositions obtainable by applying standard chemical engineering techniques, such as catalysis, hydrolysis, hydrogenation, pressure, temperature and depolymerisation to lignins, lignocellulosic biomasses in particular, which compound mixtures can be directly used as additives for epoxy-resins, phenol-formaldehyde resins, polyurethanes and flame retardants and as starting materials or intermediary compositions in the production of epoxy-resins, lignin-formaldehyde resins, polyurethanes and flame retardants.

### BACKGROUND OF THE INVENTION

Aromatic molecules are key compounds in chemical industry as they are used to produce a range of important chemicals and polymers. Their fossil-based nature drives the search for renewable alternatives. In this context, lignin is regarded as a promising sustainable alternative for fossil-based aromatics, as it is derived from lignocellulosic material. Lignocellulosic material includes, but is not limited to, wood, wood sawdust, wood chips, timber, waste wood, bark, genetically engineered wood, herbaceous crops, corn stover, straw, flax shives, sugar cane bagasse, and brewery spent grain.

Lignin, as found in the lignocellulosic material, is considered to be a structurally complex amorphous, aromatic polymer formed by radical polymerization, resulting in a polymer with different types of inter-phenolic linkages, including, but not limited to the so called β-O-4, β-5 phenylcoumarane, β-β resinol, and β-1 resinol inter-unit linkage.

To enable valorization of lignin, this native lignin material is often extracted from the lignocellulosic material, whereby its molecular structure is altered. During such extractions, the lignin is often degraded, resulting in higher molecular weight lignin fragments with less interesting molecular structures, which impedes valorization and/or results in lower-value lignin products.

A particular lignin structure is the resulting lignin from the pulp and paper industry. This lignin can be regarded as a highly degraded lignin, which has a low value for further valorization and is primary incinerated for energy recuperation. Typically, this lignin has little native β-O-4 in its structure, as under the applied conditions the β-O-4 is being cleaved, while many new—and unknown—carbon-carbon linkages are being formed. Besides, part of the other native inter-unit linkages are either conserved or converted into other newly formed linkages. This results in the production of high molecular weight lignin fragments, of which a part of the present molecules in this lignin have a molecular weight higher than 10000 g/mol (P. C. A. Bruijnincx et al., Green Chemistry, 2016, volume 18, pages 2651-2665).

Another particular lignin structure is the resulting lignin from so called organosolv processes, as disclosed in US20160024712A1. Also this resulting organosolv lignin has a degraded structure with little native β-O-4 in its structure and many new, unknown carbon-carbon linkages. Besides, part of the other native inter-unit linkages are either conserved or converted into other newly formed linkages. As a result, high molecular weight lignin fragments with low functionality are present in this lignin, of which a part of the present molecules in this lignin have a molecular weight higher than 10000 g/mol (P. C. A. Bruijnincx et al., Green Chemistry, 2016, 18, 2651-2665).

Yet another lignin structure is the so-called hydrolysis-lignin, which is a byproduct from the emerging cellulosic bioethanol industry. In this hydrolysis-lignin, the native lignin structure is partly retained and partly degraded. Thus, it still contains a considerable amount of native lignin inter-unit linkages, resulting in a low phenolic functionality as part of the phenolic units remain etherified (S. J. Horn et al. Biotechnology for Biofuels, 2018, volume 11, article 61).

Yet another lignin structure is the resulting lignin from the so-called acetal-stabilized lignin, as disclosed in US20210107851A1. This acetal stabilized lignin typically has a lignin structure resembling the native lignin structure, as the native β-O-4 is protected by acetal formation. Moreover, most of the other native inter-unit linkages are conserved. As a result, this lignin has a high molecular weight, of which part of the present molecules in this lignin have a molecular weight higher than 10000 g/mol, and a very low phenolic functionality as most of the phenolic units are etherified (J. S. Luterbacher et al., Chemical Science, 2019, volume 10, pages 8135-8142).

Yet another lignin structure is the resulting lignin from the so-called high alcohol organosolv process (J. S. Luterbacher et al., Nature Reviews Chemistry, 2020, 4, 311-330). This alcohol stabilized lignin typically has a lignin structure resembling the native lignin structure, as the native β-O-4 is protected by α-alkoxylation. Moreover, most of the other native inter-unit linkages are conserved. Also this lignin contains molecules with a high molecular weight and a very low phenolic functionality, as most of the phenolic units remain etherified (K. Barta, Green Chemistry, 2017, volume 19, pages 2774-2782).

There is a need for more functional and low molecular weight lignin structures, directly derived from lignocellulosic biomass, possessing a high number of reactive molecular groups, such as aromatic and/or phenolic hydroxyl functionalities. Such functionalities can be employed to synthesize value added chemicals starting from these engineered lignin structures. A particular interesting example is the incorporation of phosphorus into the lignin backbone. Indeed, phosphorylated lignin has the potential to be used as a biobased flame retardant, wherein the amount of phosphorous incorporated in the lignin structure is the pivotal step as it adds to the flame retardancy of the final product. Another particular interesting example is the use of such high functional, low molecular weight lignin structures as a polymer precursor, wherein the density of the reactive polymer groups can play an important role for the final polymer properties. A higher content of specific functional groups thus impacts the valorization potential of lignin.

Therefore, there is an unmet need for an engineered, low molecular weight, highly functional lignin, which can be used as a precursor in the production of a wide variety of lignin-based products.

### SUMMARY OF THE INVENTION

The objective technical problem solved by the present invention is the realization of highly reactive engineered phenolic compound compositions, which can be used as a precursor in the synthesis of a wide variety of lignin-based products.

An advantage of the present invention is the use of highly functional low molecular weight lignin as a starting material or intermediate include, but are not limited to, the production of thermoplastic polymers, thermosetting polymers, polymer additives, flame retardants, additives and antioxidants.

Another advantage of the phenolic compound compositions according to the present invention is an increased reactivity in comparison with prior art phenolic compound compositions derived from lignin or lignocellulose conversion techniques.

Another advantage of the phenolic compound compositions according to the present invention is the higher number of aliphatic hydroxyl groups.

Another advantage of the phenolic compound compositions according to the present invention is an increased covalent urethane bond formation upon reaction with isocyanates opening the possibility of more densely crosslinked polyurethanes.

Another advantage of the phenolic compound compositions according to the present invention is a higher quantity of covalently bound phosphorus.

According to a first aspect of the present invention an engineered composition comprising aromatic compounds is provided, whereby the molecular mass of the aromatic compounds is between 90 g/mol and 10000 g/mol, wherein the aromatic compounds comprise at least one aromatic compound selected from the formulae

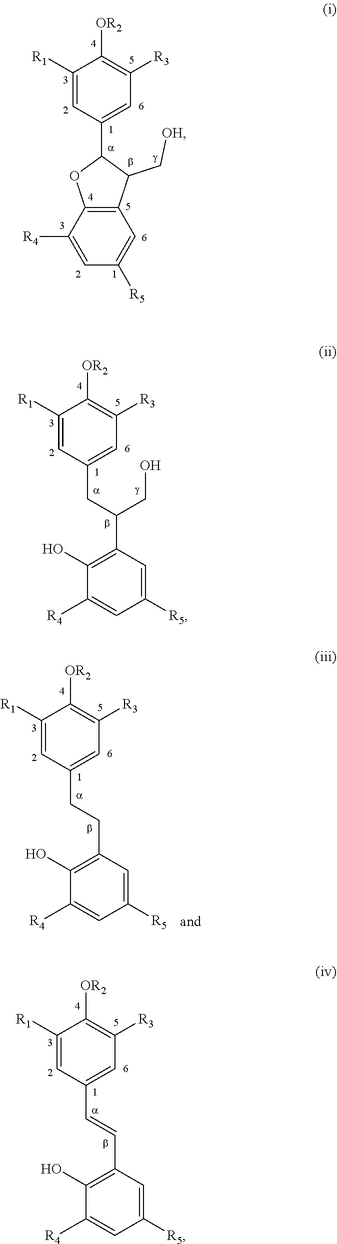

whereof the molecular ratio of ((ii)+(iii))/((i)+(ii)+(iii)+(iv)) is higher than 0.1, wherein each of R₁, R₃, and R₄ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and wherein the aromatic compounds comprise at least one aromatic compound selected from the formulae

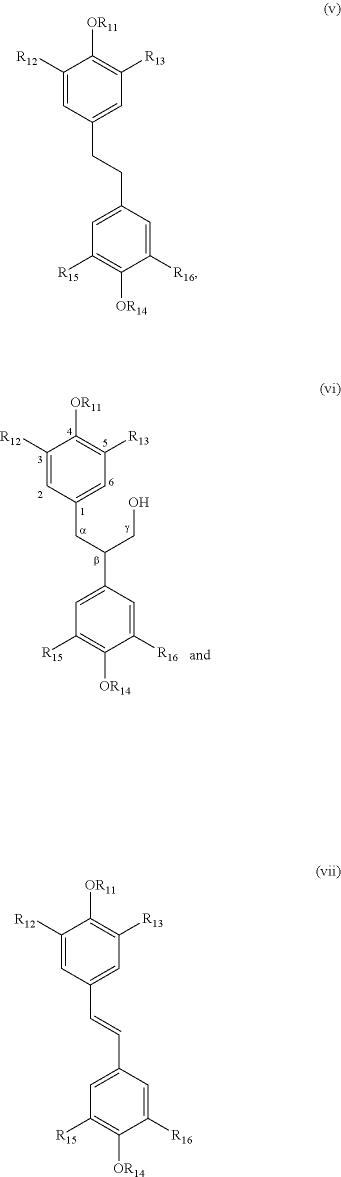

whereof the molecular ratio of ((v)+(vi))/((vi)+(vi)+(vii)) is higher than 0.15, wherein each of R₁₂, R₁₃, R₁₅ and R₁₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, wherein each of R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and wherein at the aromatic compounds comprise at least one aromatic compound selected from the formulae according to

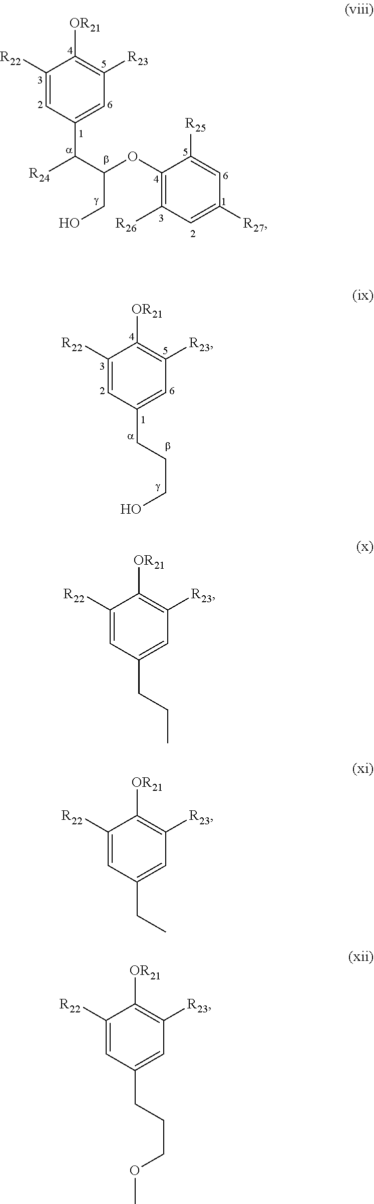

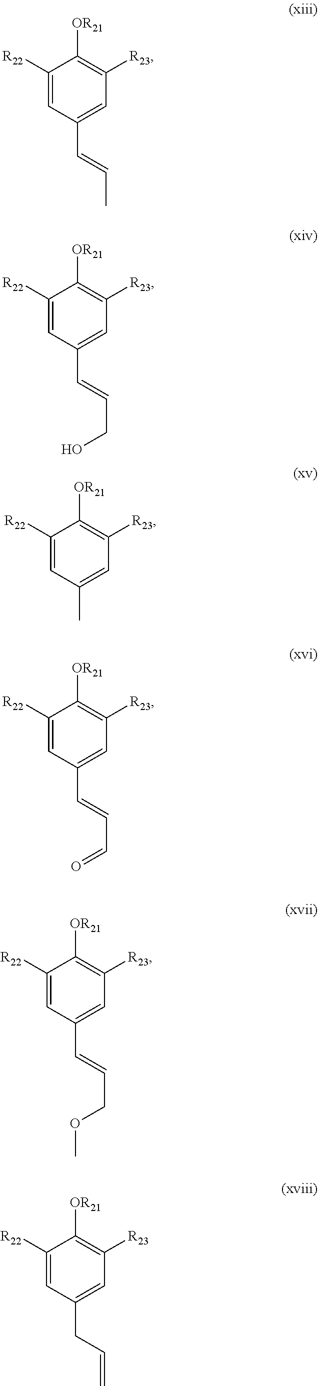

and whereby the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.5, wherein each of R₂₂, R₂₃, R₂₅ and R₂₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer, wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, wherein R₂₄ is independently chosen from —H, —OH, or —O-Alkyl wherein the alkyl group is derived from the alcohol solvent of the process, wherein R₂₇ is independently chosen from —H, a —O-4 linkage to an aromatic monomer or aromatic oligomer, a β-5 linkage to an aromatic monomer or aromatic oligomer, a 1-3 linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CH—CH₂O(CH₂)₃CH₃, a carbon linkage to an aromatic monomer or an aromatic oligomer.

According to a second aspect of the present invention a non-naturally occurring composition is provided, according to the first aspect of the present invention.

According to a third aspect of the present invention an additive for resins is provided, according to the first aspect of the present invention.

According to a fourth aspect of the present invention an additive for epoxy-resins is provided, according to the first aspect of the present invention.

According to a fifth aspect of the present invention an additive for phenol-formaldehyde resins is provided, according to the first aspect of the present invention.

According to a sixth aspect of the present invention an intermediary composition in the production of resins is provided, according to the first aspect of the present invention.

According to a seventh aspect of the present invention an intermediary composition in the production of epoxy-resins is provided, according to the first aspect of the present invention.

According to an eighth aspect of the present invention an intermediary composition in the production of lignin-formaldehyde resins is provided, according to the first aspect of the present invention.

According to a ninth aspect of the present invention a starting material in the production of resins is provided, according to the first aspect of the present invention.

According to a tenth aspect of the present invention a starting material in the production of epoxy-resins is provided, according to the first aspect of the present invention.

According to an eleventh aspect of the present invention a starting material in the production of lignin-formaldehyde resins is provided, according to the first aspect of the present invention.

According to a twelfth aspect of the present invention an intermediary composition in the production of polyurethanes is provided, according to the first aspect of the present invention.

According to a thirteenth aspect of the present invention an intermediary composition in the production of flame retardants is provided, according to the first aspect of the present invention.

According to a fourteenth aspect of the present invention a starting material in the production of polyurethanes is provided, according to the first aspect of the present invention.

According to a fifteenth aspect of the present invention a starting material in the production of flame retardants is provided, according to the first aspect of the present invention.

According to a sixteenth aspect of the present invention, a catalytic process for producing aromatic compound compositions from lignin biomass is provided by dispersing the biomass with a catalyst in an alcohol or alcohol/water solvent in a pressurisable container, providing a hydrogen gas pressure greater than 1 bar at room temperature in said container and heating said dispersion to at least 150° C. and heating at said temperature for at least 0 minutes, wherein said catalyst comprises at least one metal selected from the group consisting of ruthenium, palladium, nickel, copper, platinum, iridium, rhodium, cobalt, iron and osmium.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed. Some statements of the invention are set forth in claim format directly below:

1) An engineered composition comprising aromatic compounds, whereby the molecular mass of the aromatic compounds is between 90 g/mol and 10000 g/mol, wherein the aromatic compounds comprise at least one aromatic compound selected from the formulae

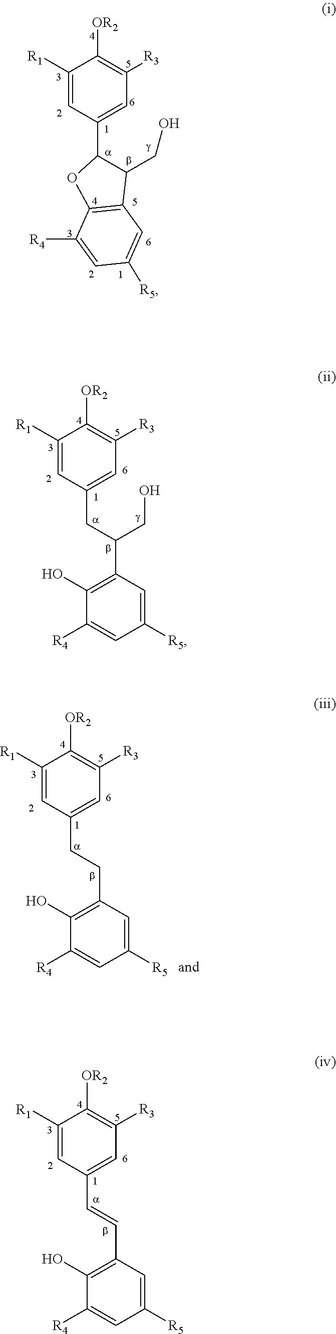

whereby the molecular ratio of ((ii)+(iii))/((i)+(ii)+(iii)+(iv)) is higher than 0.1, wherein each of R₁, R₃, and R₄ is independently chosen from —H, —OH, —O—CH₃, a 4-O-5 linkage to an aromatic monomer or aromatic oligomer, a 5-5 linkage to an aromatic monomer or aromatic oligomer, a 4-O-5 linkage to an aromatic monomer or aromatic oligomer, a carbon linkage to an aromatic monomer or aromatic oligomer, or a carbon-oxygen linkage to an aromatic monomer or aromatic oligomer, wherein R_{z} is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer and wherein R₅ is selected from —H, a β-O-4 linkage to an aromatic monomer or aromatic oligomer, a 13-5 linkage to an aromatic monomer or aromatic oligomer, a β-β linkage to an aromatic monomer or aromatic oligomer, a β-1 linkage to an aromatic monomer or aromatic oligomer, an ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and wherein the aromatic compounds comprise at least one aromatic compound selected from the formulae

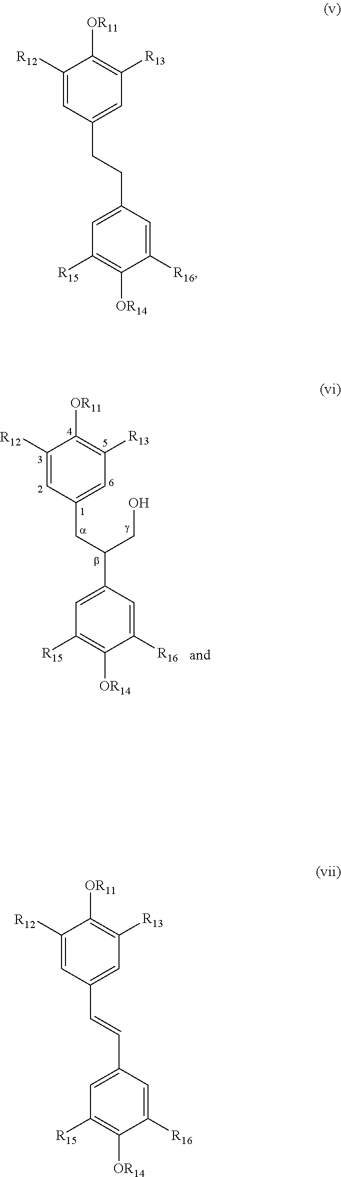

and whereby the molecular ratio of ((v)+(vi))/((v)+(vi)+(vii)) is higher than 0.15, wherein each of R₁₂, R₁₃, R₁₅ and R₁₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer and wherein each of R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer; and wherein the aromatic compounds comprise at least one aromatic compound selected from the formulae

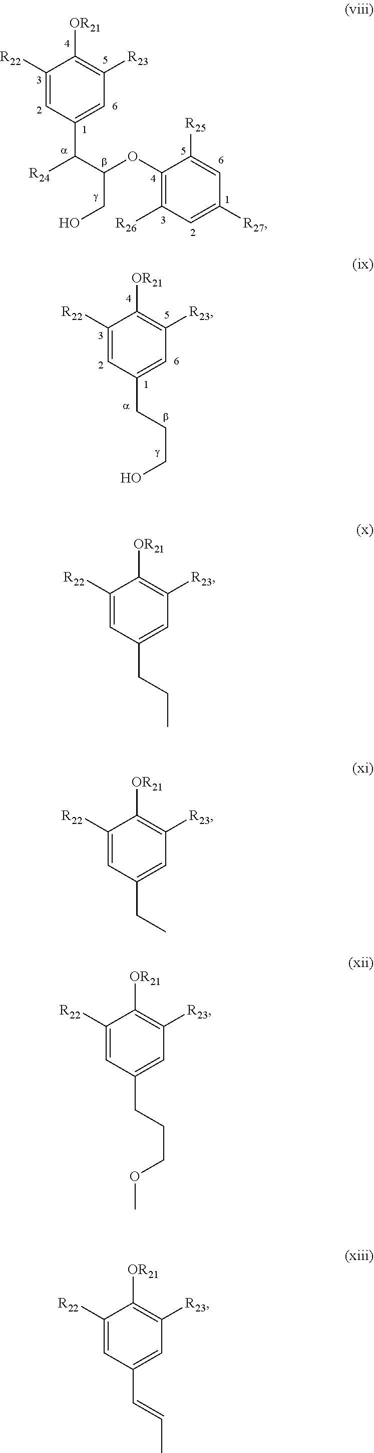

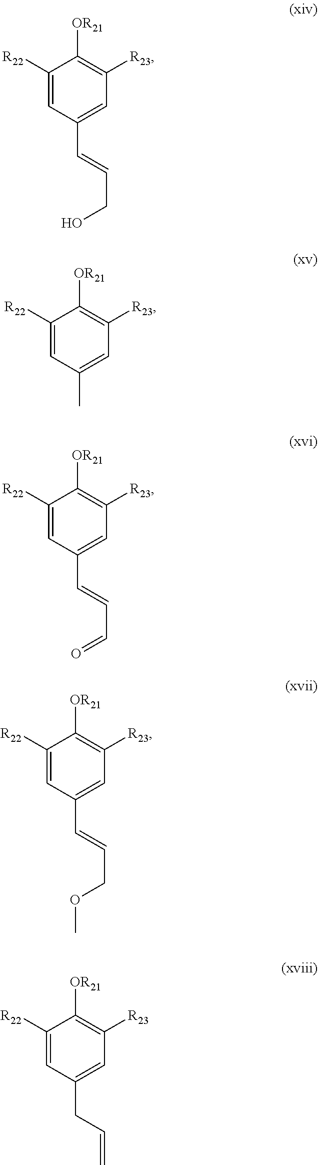

and whereby the molecular ratio of ((ix)+(x)+(xi)+(xii))/((viii)+(ix)+(x)+(xi)+(xii)+(xiiii)+(xiv)+(xv)+(xvi)+(xvii)+(xviii)) in the aromatic mixture is higher than 0.5, wherein each of R₂₂, R₂₃, R₂₅ and R₂₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer and wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer and wherein R₂₄ is independently chosen from —H, —OH, or —O-Alkyl wherein the alkyl group is derived from the alcohol solvent of the process and wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

2) The engineering composition according to statement 1, characterized in that R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an ‘end-unit’ selected of CH3, CH2-CH3, (CH2)2CH3, CH2CHCH2, (CH)2CH3, (CH2)2CH2OH, (CH2)2CHO, (CH)2CH2OH, (CH2)2CH2OCH3, (CH)2)CH2OCH3, (CH2)CH2OCH2CH3, (CH)CH2OCH2CH3, (CH2)CH2O(CH2)2CH3, (CH)CH2O(CH2)2CH3, (CH2)CH2OCH(CH3)2, (CH)CH2OCH(CH3)2, (CH2)CH2O(CH2)3CH3, (CH)CH2O(CH2)3CH3, or a carbon linkage to an aromatic monomer or aromatic oligomer.

3.) The engineering composition according to statement 1 or 2, wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected of CH3, CH2-CH3, (CH2)2CH3, CH2CHCH2, (CH)2CH3, (CH2)2CH2OH, (CH2)2CHO, (CH)2CH2OH, (CH2)2CH2OCH3, (CH)2)CH2OCH3, (CH2)CH2OCH2CH3, (CH)CH2OCH2CH3, (CH2)CH2O(CH2)2CH3, (CH)CH2O(CH2)2CH3, (CH2)CH2OCH(CH3)2, (CH)CH2OCH(CH3)2, (CH2)CH2O(CH2)3CH3, (CH)CH2O(CH2)3CH3, a carbon linkage to an aromatic monomer or an aromatic oligomer.

4.) The engineered composition according to any of the preceding statements, characterized in that the aromatic compounds comprise at least one aromatic compound selected from the formulae

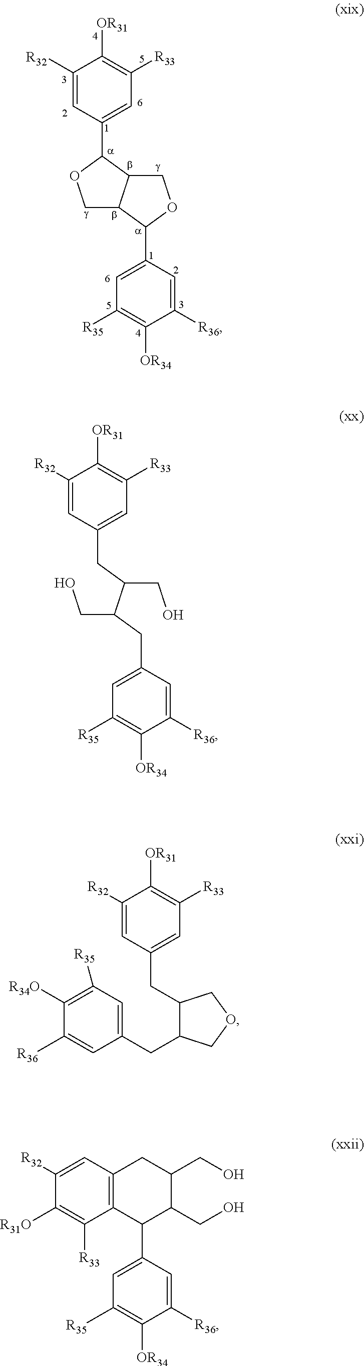

and whereby the molecular ratio of ((xx)+(xxi)+(xxii))/((xix)+(xx)+(xxi)+(xxii)) is higher than 0.25, wherein each of R₃₂, R₃₃, R₃₅ and R₃₆ is independently chosen from —H, —OH, —O—CH₃, 4-O-5 linkage to an aromatic monomer or aromatic oligomer, a 5-5 linkage to aromatic monomer or aromatic oligomer, a β-5 linkage to an aromatic monomer or aromatic oligomer, a carbon linkage to an aromatic monomer or aromatic oligomer, a carbon-oxygen linkage to an aromatic monomer or aromatic oligomer and wherein each of R₃₁ and R₃₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer.

5) The engineered composition according to any one of the statements 1 to 4, characterized in that this composition is composed of aromatic compounds, whereby the molecular mass of the aromatic compounds is between 90 g/mol and 10000 g/mol.

6) The engineered composition according to any one of the statements 1 to 4, characterized in that this composition is consisting of aromatic compounds, whereby the molecular mass of the aromatic compounds is between 90 g/mol and 10000 g/mol.

7) The engineered composition according to any one of the statements 1 to 6, wherein the aromatic compounds comprise at least one aromatic compound selected from the formulae

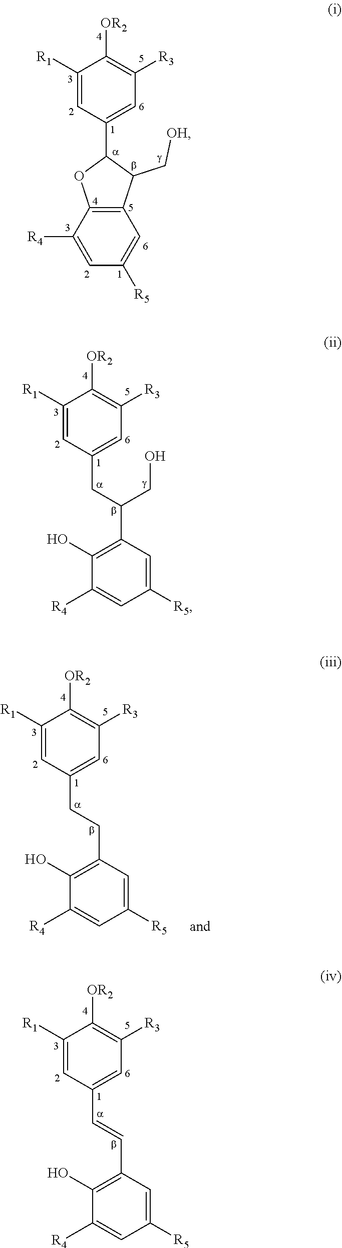

and whereby the molecular ratio of ((ii)+(iii))/((i)+(ii)+(iii)+(iv)) is higher than 0.7

8) The engineered composition to any one of the statements 1 to 6, wherein the aromatic compounds comprise at least one aromatic compound selected from the formulae

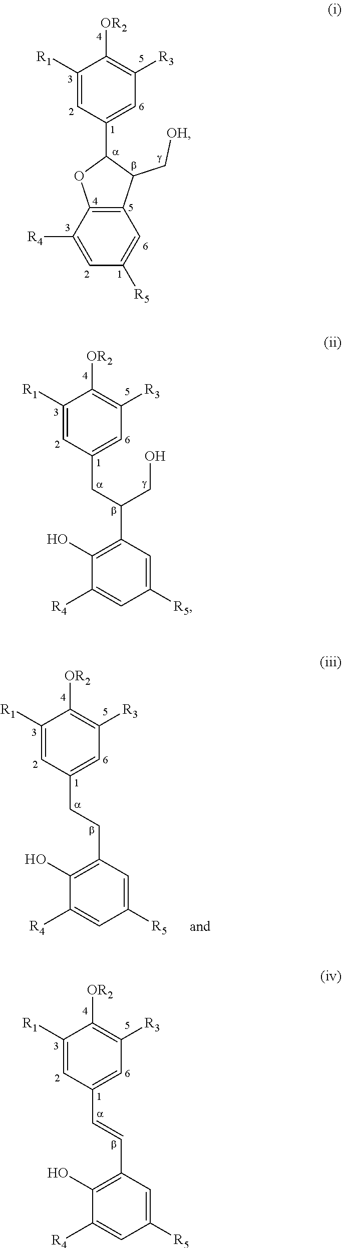

and whereby the molecular ratio of ((ii)+(iii))/((i)+(ii)+(iii)+(iv)) is higher than 0.9.

9) The engineered composition to any one of the statements 1 to 8, wherein the aromatic compounds comprise at least one aromatic compound selected from the formulae

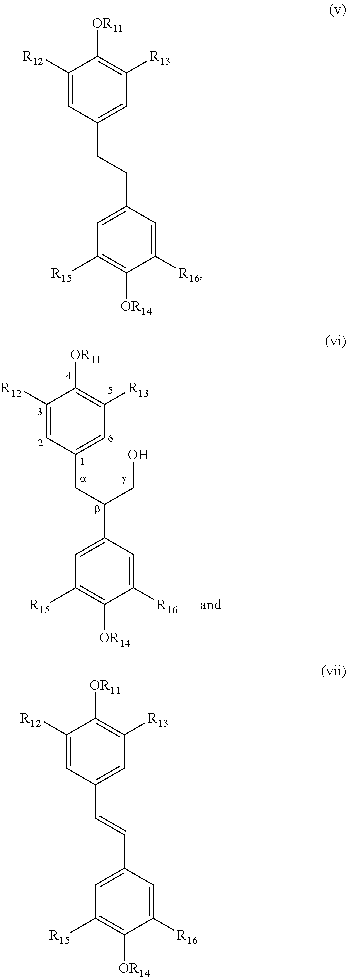

and whereby the molecular ratio of ((v)+(vi))/((v)+(vi)+(vii)) is higher than 0.6.

10) The engineered composition to any one of the statements 1 to 8, wherein the aromatic compounds comprise at least one aromatic compound selected from the formulae

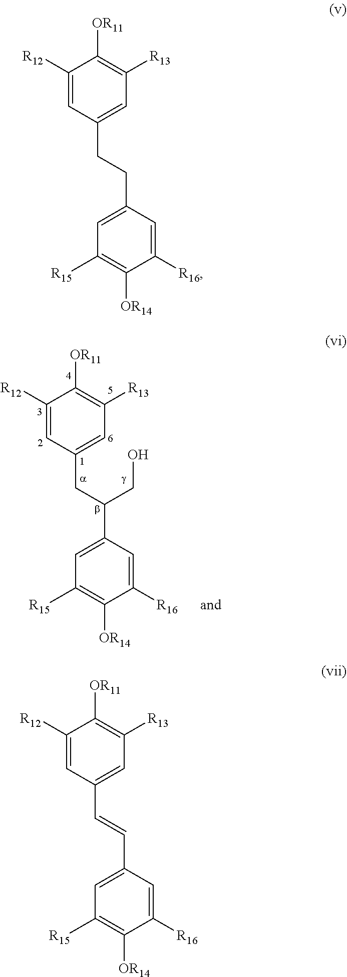

and whereby the molecular ratio of ((v)+(vi))/((v)+(vi)+(vii)) is higher than 0.9

11) The engineered composition to any one of the statements 1 to 10, wherein the aromatic compounds comprise at least one aromatic compound selected from the formulae

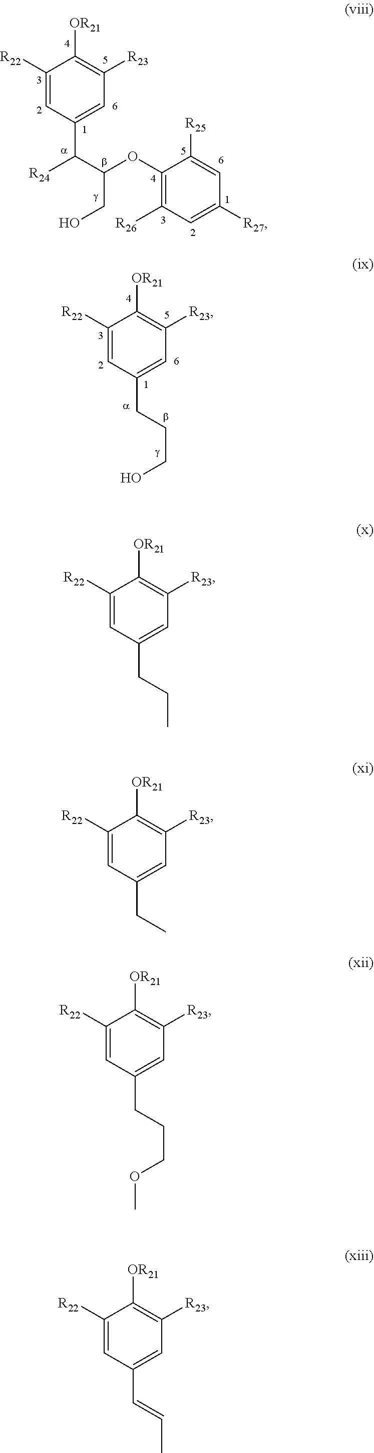

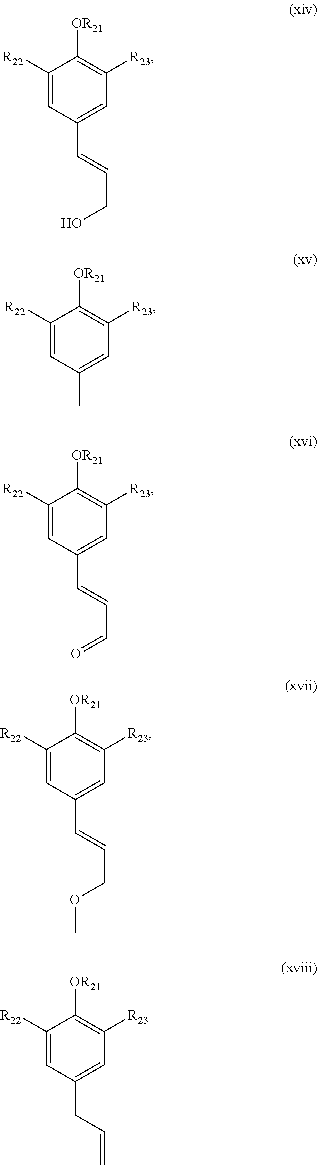

and whereby the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.7

12) The engineered composition to any one of the statements 1 to 10, wherein the aromatic compounds comprise at least one aromatic compound selected from the formulae

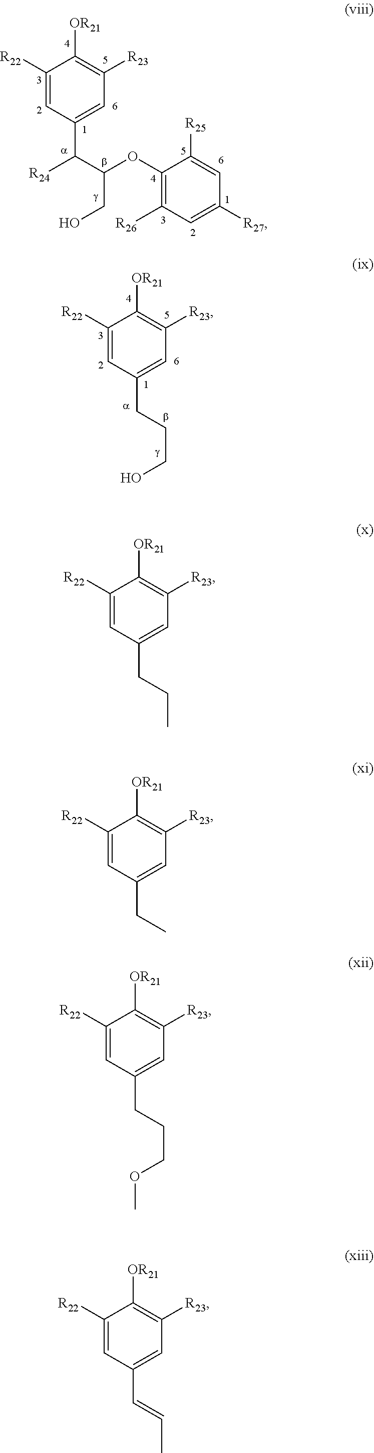

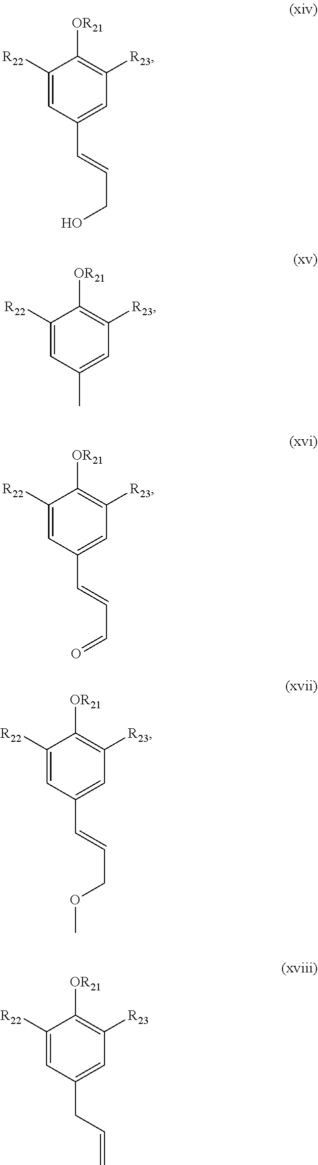

and whereby the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.9

13) The engineered composition to any one of the statements 1 to 12, wherein the aromatic compounds comprise at least one aromatic compound selected from the formulae

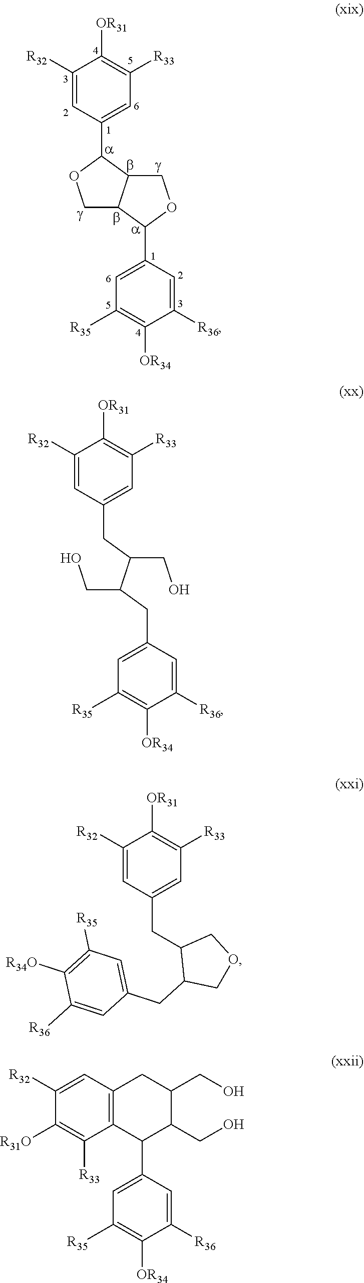

and whereby the molecular ratio of ((xx)+(xxi)+(xxii))/((xix)+(xx)+(xxi)+(xxii)) is higher than 0.5

14) The engineered composition to any one of the statements 1 to 12, wherein the aromatic compounds comprise at least one aromatic compound selected from the formulae

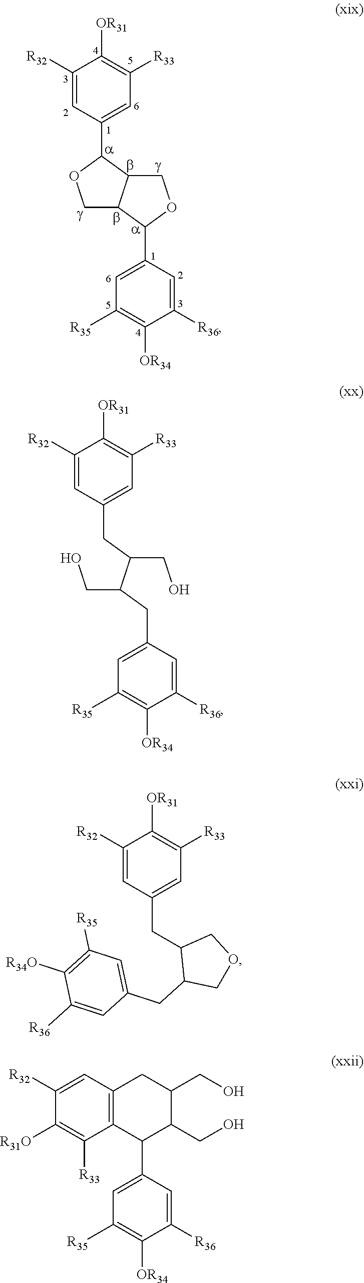

and whereby the molecular ratio of ((xx)+(xxi)+(xxii))/((xix)+(xx)+(xxi)+(xxii)) is higher than 0.9

15) The engineered composition according to any one of the preceding statements 1 to 14, which is a lignin degradation mixture.

16) The engineered composition according to any one of the preceding statements 1 to 14, whereby the aromatic compounds are lignin derived aromatic compounds.

17) The engineered composition according to any one of the preceding statements 1 to 14, which is a lignin conversion in lignin derived aromatic compounds.

18) The engineered composition according to any one of the preceding statements 1 to 14, which is a mixture with lignin derived aromatic compounds from catalytic degradation of lignocellulose.

19) The engineered composition according to any one of the preceding statements 1 to 14, which is an engineered catalytic degradation product of lignocellulose.

20) A non-naturally occurring composition, according to any one of the preceding statements 1 to 19.

21) An additive for resins, according to any one of the preceding statements 1 to 20.

22) An additive for epoxy-resins, according to any one of the preceding statements 1 to 20.

23) An additive for phenol-formaldehyde resins, according to any one of the preceding statements 1 to 20.

24) An intermediary composition in the production of resins, according to any one of the preceding statements 1 to 20.

25) An intermediary composition in the production of epoxy-resins, according to any one of the preceding statements 1 to 20.

26) An intermediary composition in the production of lignin-formaldehyde resins, according to any one of the preceding statements 1 to 20.

27) A starting material in the production of resins, according to any one of the preceding statements 1 to 20.

28) A starting material in the production of epoxy-resins, according to any one of the preceding statements 1 to 20.

29) A starting material in the production of lignin-formaldehyde resins, according to any one of the preceding statements 1 to 20.

30) The engineered composition according to any one of the preceding statements 1 to 14, which has a dispersity index lower than 2.5

31) The engineered composition according to any one of the preceding statements 1 to 15, which has more than 3 mmol aromatic OH per gram of said mixture and more than 1 mmol aliphatic OH per gram of said mixture.

32) The engineered composition according to any one of the preceding statements 1 to 15, which has more than 3 mmol aromatic OH per gram of said mixture and more than 3 mmol aliphatic OH per gram of said mixture

33) The engineered composition according to any one of the preceding statements 1 to 15, which has more than 4 mmol aromatic OH per gram of said mixture and more than 1 mmol aliphatic OH per gram of said mixture

34) The engineered composition according to any one of the preceding statements 1 to 15, which has more than 4 mmol aromatic OH per gram of said mixture and more than 3 mmol aliphatic OH per gram of said mixture.

35) The engineered composition according to any one of the preceding statements 1 to 34, wherein the aromatic compounds are phenols and/or phenol ethers.

36) An additive for polyurethanes, according to any one of the preceding statements.

37) An additive for flame retardants, according to any one of the statements 1 to 35.

38) An intermediary composition in the production of polyurethanes, according to any one of the statements 1 to 35.

39) An intermediary composition in the production of flame retardants, according to any one of the statements 1 to 35.

40) A starting material in the production of polyurethanes, according to any one of the statements 1 to 35.

42) A starting material in the production of flame retardants, according to any one of the statements 1 to 35.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a schematic view showing the ratio for β-5 chemical linkage, whereby the ratio β-5=(β-5 γ-OH+β-5 E)/(β-5 γ-OH+β-5 E+β-5 Stilbene+β-5 Phenylcoumaran) & ratio β-5=((ii)+(iii))/((i)+(ii)+(iii)+(iv)).

FIG. 2 is a schematic view showing the ratio for β-β chemical linkage, whereby ratio β-β=(β-β 2×γ-OH+β-β THF+β-β 2×γ-OHc)/(β-β 2×γ-OH+β-βTHF+β-β 2×γ-OHc+β-β Resinol+β-β epiresinol) & whereby ratio β-β=((xx)+(xxi)+(xxii))/((xix)+(xx)+(xxi)+(xxii))

FIG. 3 is a schematic view showing the ratio for Ratio for β-1 chemical linkage, whereby ratio β-1=(β-1 γ-OH+β-1 E)/(β-1 γ-OH+β-1 E+β-1 Stilbene) and whereby ratio β-1=((v)+(vi))/((v)+(vi)+(vii)).

FIG. 4A is a schematic view showing the Ratio 1 end-groups=[(ix)+(x)+(xi)+(xii)]/[(viii)+(ix)+(x)+(xi)+(xii)+(xiii)+(xiv)+(xv)+(xvi)+(xvii)+(xviii)]

FIG. 4B is a schematic view showing the Ratio 2 end-groups=[(ix)]/[(viii)+(ix)+(x)+(xi)+(xii)+(xiii)+(xiv)+(xv)+(xvi)+(xvii)+(xviii)]

FIG. 4C is a schematic view showing the Ratio 3 end-groups=[(viii)]/[(viii)+(ix)+(x)+(xi)+(xii)+(xiii)+(xiv)+(xv)+(xvi)+(xvii)+(xviii)]

FIG. 4D is a schematic view showing the Ratio 4 end-groups=[(x)]/[(viii)+(ix)+(x)+(xi)+(xii)+(xiii)+(xiv)+(xv)+(xvi)+(xvii)+(xviii)]

FIG. 5. displays the relative abundance of molecular structures of β-1 in the compositions of comparative examples 1-3

FIG. 6. displays the relative abundance of molecular structures of β-5 in the compositions of comparative examples 1-3

FIG. 7. displays the relative abundance of molecular structures of β-β in the compositions of comparative examples 1-3

FIG. 8. displays the relative abundance of end-group ratios in the compositions of comparative examples 1-3

FIG. 9. displays the relative abundance of molecular structures of β-1 in the compositions of comparative examples 4-6

FIG. 10. displays the relative abundance of molecular structures of β-5 in the compositions of comparative examples 4-6

FIG. 11. displays the relative abundance of molecular structures of β-β in the compositions of comparative examples 4-6

FIG. 12. displays the relative abundance of end-group ratios in the compositions of comparative examples 4-6

FIG. 13. displays the relative abundance of molecular structures of β-1 in the compositions of invention examples 1-5

FIG. 14. displays the relative abundance of molecular structures of β-5 in reactions in the compositions of invention examples 1-5

FIG. 15. displays the relative abundance of molecular structures of β-β in reactions in the compositions of invention examples 1-5

FIG. 16. displays the relative abundance of end-group ratios in reactions in the compositions of invention examples 1-5

FIG. 17. displays the relative abundance of molecular structures of β-1 in the compositions of invention examples 5-11

FIG. 18. displays the relative abundance of molecular structures of β-5 in the compositions of invention examples 5-11

FIG. 19. displays the relative abundance of molecular structures of β-β in the compositions of invention examples 5-11

FIG. 20. displays the relative abundance of end-group ratios in the compositions of invention examples 5-11

FIG. 21. displays the relative abundance of molecular structures of β-β in the compositions of invention examples 12-16

FIG. 22. displays the relative abundance of molecular structures of β-1 in the compositions of invention examples 12-16

FIG. 23. displays the relative abundance of molecular structures of β-5 in the compositions of invention examples 12-16

FIG. 24. displays the relative abundance of end-group ratios in the compositions of invention examples 12-16

FIG. 25. displays the relative abundance of molecular structures of β-1 in the compositions of invention examples 17-21

FIG. 26. displays the relative abundance of molecular structures of β-5 in the compositions of invention examples 17-21

FIG. 27. displays the relative abundance of molecular structures of β-β in the compositions of invention examples 17-21

FIG. 28. displays the relative abundance of end-group ratios in the compositions of invention examples 17-21

FIG. 29. displays the relative abundance of molecular structures of β-1 in the compositions of invention examples 27-30

FIG. 30. displays the relative abundance of molecular structures of β-5 in the compositions of invention examples 27-30

FIG. 31. displays the relative abundance of molecular structures of β-β in the compositions of invention examples 27-30

FIG. 32. displays the relative abundance end-group ratios in the compositions of invention examples 27-30

FIG. 33. displays the relative abundance of molecular structures of β-1 in the compositions of invention examples 22-26

FIG. 34. displays the relative abundance of molecular structures of β-5 in the compositions of invention examples 22-26

FIG. 35. displays the relative abundance of molecular structures of β-β in the compositions of invention examples 22-26

FIG. 36. displays the relative abundance of end-group ratios in the compositions of invention examples 22-26

FIG. 37. displays the relative abundance of molecular structures of β-1 in the compositions of invention examples 31-34

FIG. 38. displays the relative abundance of molecular structures of β-5 in the compositions of invention examples 31-34

FIG. 39. displays the relative abundance of molecular structures of β-β in the compositions of invention examples 31-34

FIG. 40. displays the relative abundance of end-group ratios in the compositions of invention examples 31-34

FIG. 41. displays the relative abundance of molecular structures of β-1 in the compositions of invention examples 34 and 40-42

FIG. 42. displays the relative abundance of molecular structures of β-5 in the compositions of invention examples 34 and 40-42

FIG. 43. displays the relative abundance of molecular structures of β-β in the compositions of invention examples 34 and 40-42

FIG. 44. displays the relative abundance end-group ratios in the compositions of invention examples 34 and 40-42

FIG. 45. displays the relative abundance of molecular structures of β-1 in the compositions of invention examples 35-39

FIG. 46. displays the relative abundance of molecular structures of β-5 in the compositions of invention examples 35-39

FIG. 47. displays the relative abundance of molecular structures of β-β in the compositions of invention examples 35-39

FIG. 48. displays the relative abundance end-group ratios in the compositions of invention examples 35-39

FIG. 49. displays the relative phenolic and aliphatic OH content of the compositions of invention example 5 (Pd/C), invention example 21 (Ru/C), invention example 34 (Ni—Al₂O₃) and comparative example 3 (No catalyst)

FIG. 50. displays the relative phenolic and aliphatic OH content of the mixtures of invention example 16 (Pd/C), invention example 26 (Ru/C), invention example 39 (Ni—Al₂O₃) and comparative example 6 (No catalyst)

FIG. 51. Gel Permeation Chromatogram of the compositions of invention examples 1-5.

FIG. 52. Gel Permeation Chromatogram of the compositions of invention examples 12-16.

FIG. 53. Gel Permeation Chromatogram of the compositions of invention examples 17-21.

FIG. 54. Gel Permeation Chromatogram of the compositions of invention examples 22-26.

FIG. 55. Gel Permeation Chromatogram of the compositions of invention examples 31-34.

FIG. 56. Gel Permeation Chromatogram of the compositions of invention examples 35-39

### DETAILED DESCRIPTION

### Detailed Description of Embodiments of the Invention

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to the devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Other embodiments of the invention will be apparent to those skilled in the art from consideration of the specification and practice of the invention disclosed herein.

It is intended that the specification and examples be considered as exemplary only.

Each and every claim is incorporated into the specification as an embodiment of the present invention. Thus, the claims are part of the description and are a further description and are in addition to the preferred embodiments of the present invention.

Each of the claims set out a particular embodiment of the invention.

The following terms are provided solely to aid in the understanding of the invention.

### Definitions

It is to be understood that the terminology used herein for the purpose of describing particular aspects only and is not intended to be limiting. As used in the specification and in the claims, the term “comprising” can include the aspects “consisting of” and “consisting essentially of”. Unless defined otherwise, all technical and scientific terms used herein have the same meaning as commonly understood by one or ordinary skill in the art which this invention belongs. In this specification and in the claims which follow, reference will be made to a number of terms which shall be defined herein.

In the present invention, the term “engineered composition” means a composition whose composition is predictably adjusted by varying (tuning) its preparation condition.

In the present invention, the term “biomass” is used for the term “lignocellulosic material” and lignocellulosic material may be in the meaning of lignocellulose or material comprising lignocellulose.

In the present invention, the term “aromatic monomer” means molecules with one aromatic group. The mixture comprises molecules or compounds that result from the chemical modification of lignin. Hence the minimal starting material is lignin or a material that comprises lignin. Hence these molecules or compounds can be referred to as “lignin-derived monophenolics”, “lignin-derived monomers”, “lignin monomers”, “phenolic monomers”, “aromatic monomers”, or “lignin-derived aromatic monomers”. These terms are used interchangeably. Chemical modification herein means depolymerisation and/or hydrogenolysis and/or decarbonylation and/or hydrolysis and/or dehydrogenation and/or partial reduction The lignin-derived monophenolics comprise compounds having the formula of

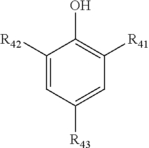

wherein each of R₄₁ and R₄₂ is independently chosen from —H, —OH or —OCH₃, and R₄₃ is chosen from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CH—CH₂O(CH₂)₃CH₃—CH₃, with selection from —CH₃, CH₂—CH₃, (CH₂)₂CH₃, —CH₂CHCH₂, —(CH)₂CH₃, (CH₂)₂CH₂OH, (CH₂)₂CHO, (CH)₂CH₂OH, (CH₂)₂CH₂OCH₃, (CH)₂)CH₂OCH₃, (CH₂)₂CH₂OCH₂CH₃, (CH)₂)CH₂OCH₂CH₃, CH₂)₂CH₂O(CH₂)₂CH₃, (CH)₂)CH₂O(CH₂)₂CH₃, CH₂)₂CH₂OCH(CH₃)₂, (CH)₂)CH₂OCH(CH₃)₂, CH₂)₂CH₂O(CH₂)₃CH₃ and (CH)₂)CH₂O(CH₂)₃CH₃ being preferred. Phenolic monomers wherein R₂ is —H and R₃ is —OCH₃ are referred to as guaiacols, abbreviated with G. Phenolic monomers wherein R₂ is —OCH₃ and R₃ is —OCH₃ are referred to as syringols, abbreviated with S.

In the present invention, the term “aromatic oligomers” means molecules with two or more aromatic centers chemically linked to each other. These aromatic oligomers result from the chemical modification of lignin. Hence, they are referred to as “lignin-derived oligomers”, “lignin-derived oligoaromatics”, ‘lignin-derived aromatic oligomers”, “lignin oligomers” or “aromatic oligomers”. These terms are used interchangeably. Chemical modification herein means depolymerisation and/or hydrogenolysis and/or decarbonylation and/or hydrolysis and/or dehydrogenation and/or partial reduction.

In the present invention, the term ‘aromatic mixture’ refers to a mixture of lignin monomers and lignin oligomers.

In the present invention, the term “dispersity index” is the ratio of the weight average molecular weight, M_{w}, over the number average molecular weight, Mₙ, and is a measure of the width of the molecular weight distribution.

In the present invention, methanol is abbreviated as MeOH, ethanol as EtOH, n-butanol as BuOH, ethyl acetate as EtOAc and tetrahydrofuran as THF.

The chemical linkages between two aromatic centers in the lignin oligomers can be divided in different groups.

The first group of chemical linkages between two aromatic centers in the lignin oligomers is a β-β linkage wherein two aromatics are linked by a substituted 4 carbon spacer. In the present invention the following four R-β linkages can be present in the aromatic oligomers and their selectivities can be tuned.

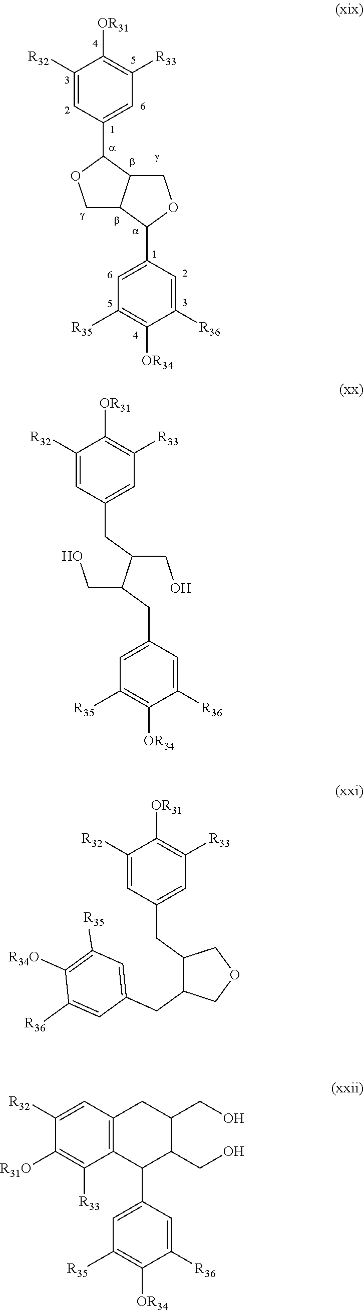

wherein each of R₃₂, R₃₃, R₃₅ and R₃₆ can be independently chosen from —H, —OH, O—CH₃, a 4-O-5 linkage to a lignin-derived monomer or lignin-derived oligomer, a 5-5 linkage to a lignin-derived monomer or lignin-derived oligomer, a β-5 linkage to a lignin-derived monomer or lignin-derived oligomer, any carbon linkage to a lignin-derived monomer or lignin-derived oligomer, any carbon-oxygen linkage to a lignin-derived monomer or lignin-derived oligomer.

Wherein each of R₃₁ and R₃₄ can be independently chosen from —H, a β-O-4 linkage to a lignin-derived monomer or lignin-derived oligomer, a 4-O-5 linkage to a lignin-derived monomer or lignin-derived oligomer, an α-O-4 linkage to a lignin-derived monomer or lignin-derived oligomer, or any carbon-oxygen linkage to a lignin-derived monomer or lignin-derived oligomer.

Linkage (xix) is also referred to as β-β resinol or β-β(xix), Linkage (xx) is also referred to as β-β 2×γ-OH or β-β(xx), Linkage (xxi) is also referred to as β-βTHF or β-β(xxi), Linkage (xxii) is also referred to as β-β 2×γ-OH condensed, β-βc 2×γ-OH, β-β 2×γ-OHc, β-β 2×γ-OHc, β-β2×c γ-OH or β-β(xxii).

The ratio β-β is defined as

$$Ratio β - β = \frac{β - β ( xx ) + β - β ( xxi ) + β - β ( xxii )}{β - β ( xix ) + β - β ( xx ) + β - β ( xxi ) + β - β ( xxii )}$$

The second group of chemical linkages between two aromatic centers is a β-5 linkage wherein two aromatics are linked by a substituted 2 carbon spacer. In the present invention the following four β-5 linkages can be present in the aromatic oligomers and their selectivities can be tuned.

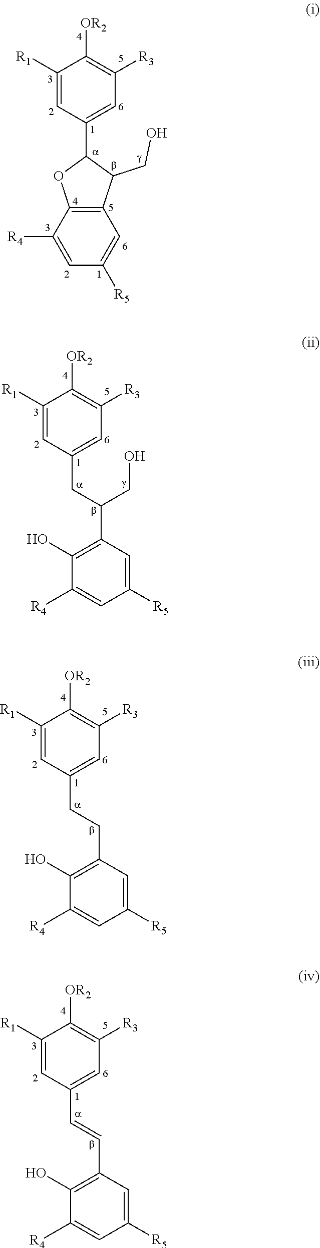

Wherein each of R₁, R₃, and R₄ can be independently chosen from —H, —OH, —O—CH₃, a 4-O-5 linkage to a lignin-derived monomer or lignin-derived oligomer, a 5-5 linkage to a lignin-derived monomer or lignin-derived oligomer, a β-5 linkage to a lignin-derived monomer or lignin-derived oligomer, any carbon linkage to a lignin-derived monomer or lignin-derived oligomer, or any carbon-oxygen linkage to a lignin-derived monomer or lignin-derived oligomer.

Wherein R₂ can be independently chosen from —H, a β-O-4 linkage to a lignin-derived monomer or lignin-derived oligomer, a 4-O-5 linkage to a lignin-derived monomer or lignin-derived oligomer, an α-O-4 linkage to a lignin-derived monomer or lignin-derived oligomer, or any carbon-oxygen linkage to a lignin-derived monomer or lignin-derived oligomer.

Wherein R₅ can be independently chosen from —H, a β-O-4 linkage to a lignin-derived monomer or lignin-derived oligomer, a β-5 linkage to a lignin-derived monomer or lignin-derived oligomer, a β-β linkage to a lignin-derived monomer or lignin-derived oligomer, a β-1 linkage to a lignin-derived monomer or lignin-derived oligomer, an ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or any carbon linkage to a lignin-derived monomer or lignin-derived oligomer.

Linkage (i) is also referred to as β-5 phenylcoumaran or β-5 (i), Linkage (ii) is also referred to as β-5 γ-OH or β-5 (ii), Linkage (iii) is also referred to as β-5 E or β-5 (iii), Linkage (iv) is also referred to as β-5 stilbene or β-5 (iv).

The ratio β-5 is defined as

$$Ratio β - 5 = \frac{( β - 5 ( ii ) + β - 5 ( iii ) )}{( β - 5 ( iv ) + β - 5 ( i ) + β - 5 ( ii ) + β - 5 ( iii ) )}$$

The third group of chemical linkages between two aromatic centers is a β-1 linkage wherein two aromatics are linked by a substituted 2 carbon spacer. In the present invention the following three β-1 linkages can be present in the aromatic oligomers and their selectivities can be tuned.

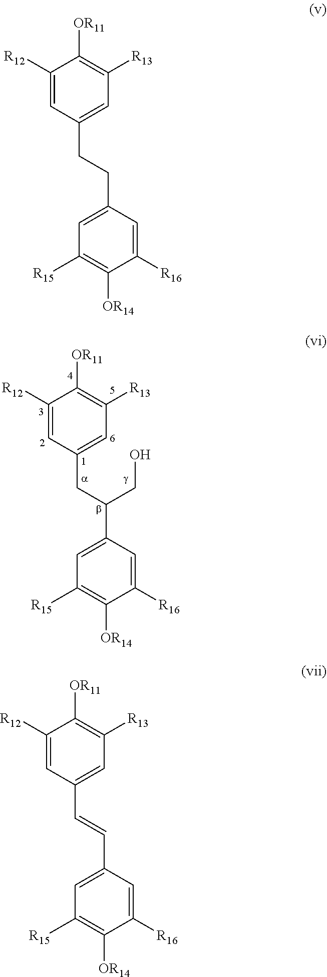

Wherein each of R₁₂, R₁₃, R₁₅ and R₁₆ can be independently chosen from —H, —OH, —O—CH₃, a 4-O-5 linkage to a lignin-derived monomer or lignin-derived oligomer, a 5-5 linkage to a lignin-derived monomer or lignin-derived oligomer, a β-5 linkage to a lignin-derived monomer or lignin-derived oligomer, any carbon linkage to a lignin-derived monomer or lignin-derived oligomer, any carbon-oxygen linkage to a lignin-derived monomer or lignin-derived oligomer.

Wherein each of R₁₁ and R₁₄ can be independently chosen from —H, a β-O-4 linkage to a lignin-derived monomer or lignin-derived oligomer, a 4-O-5 linkage to a lignin-derived monomer or lignin-derived oligomer, an α-O-4 linkage to a lignin-derived monomer or lignin-derived oligomer, or any carbon-oxygen linkage to a lignin-derived monomer or lignin-derived oligomer.

Linkage (v) is also referred to as β-1 E or β-1 (v), Linkage (vi) is also referred to as β-1 γ-OH or β-1 (vi), Linkage (vii) is also referred to as β-1 stilbene or β-1 (vii).

The ratio β-1 is defined as

$$Ratio β - 1 = \frac{β - 1 ( v ) + β - 1 ( vi )}{β - 1 ( v ) + β - 1 ( vi ) + β - 1 ( vii )}$$

The fourth group of tunable chemical linkages are β-O-4 linkages wherein two aromatics are linked by a substituted 2-carbon spacer of one aromatic on the phenolic group of the other aromatic and ‘end-units’, wherein the ‘end-units’ are various substituted aliphatics according to structures (ix)-(xviii). In the present invention the following β-O-4 linkages and ‘end units’ can be present in the aromatic monomers and oligomers and their selectivities can be tuned.

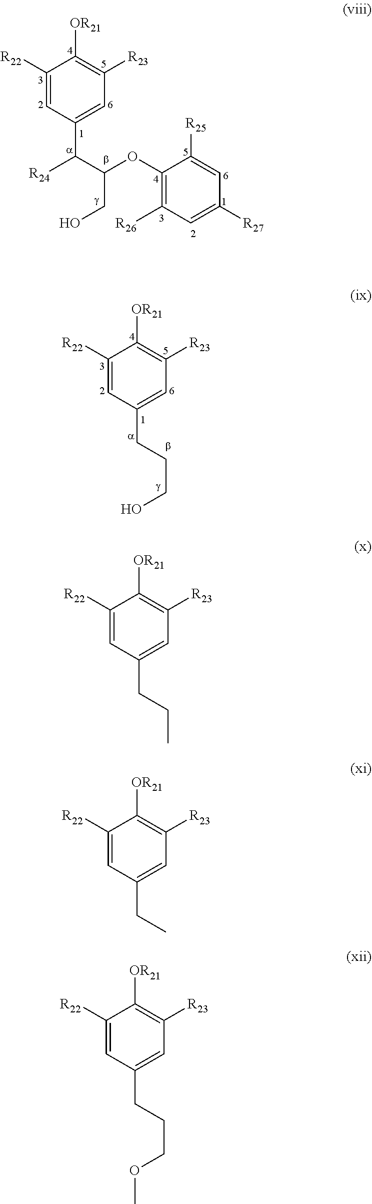

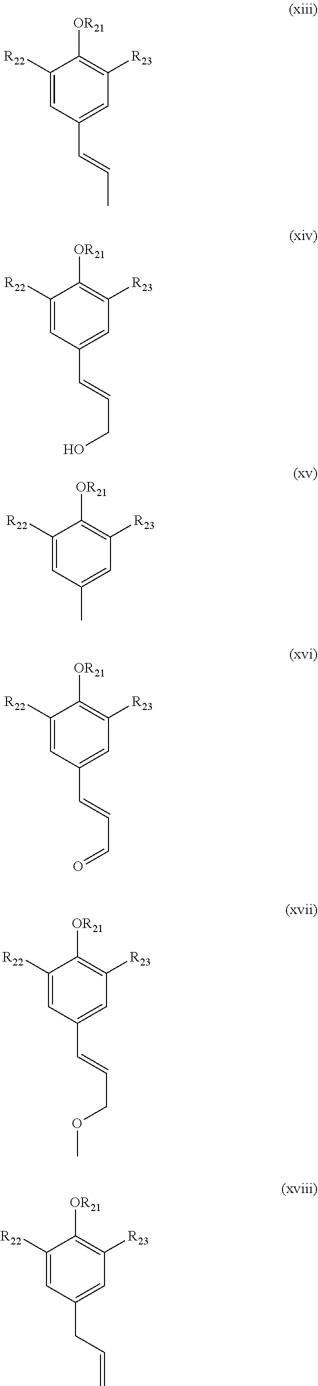

Wherein each of R₂₂, R₂₃, R₂₅ and R₂₆ can be independently chosen from —H, —OH, —O—CH₃, a 4-O-5 linkage to a lignin-derived monomer or lignin-derived oligomer, a 5-5 linkage to a lignin-derived monomer or lignin-derived oligomer, a β-5 linkage to a lignin-derived monomer or lignin-derived oligomer, any carbon linkage to a lignin-derived monomer or lignin-derived oligomer, any carbon-oxygen linkage to a lignin-derived monomer or lignin-derived oligomer.

Wherein R₂₁ can be independently chosen from —H, a β-O-4 linkage to a lignin-derived monomer or lignin-derived oligomer, a 4-O-5 linkage to a lignin-derived monomer or lignin-derived oligomer, an α-O-4 linkage to a lignin-derived monomer or lignin-derived oligomer, or any carbon-oxygen linkage to a lignin-derived monomer or lignin-derived oligomer.

Wherein R₂₄ can be independently chosen from —H, —OH, or —O-Alkyl wherein the alkyl group is derived from the alcohol solvent of the process.

Wherein R₂₇ can be independently chosen from —H, a β-O-4 linkage to a lignin-derived monomer or lignin-derived oligomer, a β-5 linkage to a lignin-derived monomer or lignin-derived oligomer, a β-β linkage to a lignin-derived monomer or lignin-derived oligomer, a β-1 linkage to a lignin-derived monomer or lignin-derived oligomer, any ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃ and any carbon linkage to a lignin-derived monomer or lignin-derived oligomer.

Linkage (viii) is also referred to as β-O-4 or End-unit (viii), Linkage (ix) is also referred to as propanol or End-unit (ix), Linkage (x) is also referred to as propyl or End-unit (x), Linkage (xi) is also referred to as ethyl or End-unit (xi), Linkage (xii) is also referred to as 3-methoxypropyl or End-unit (xii), Linkage (xiii) is also referred to as propenyl or End-unit (xiii), Linkage (xiv) is also referred to as propenol or End-unit (xiv), Linkage (xv) is also referred to as methyl or End-unit (xv), Linkage (xvi) is also referred to as propenon or End-unit (xvi), Linkage (xvii) is also referred to as 3-methoxypropenyl or End-unit (xvii), Linkage (xviii) is also referred to as methoxypropenyl or End-unit (xviii).

The ratio 1 end-groups is defined as

$$Ratio 1 end - groups = \frac{\begin{matrix}End - unit ( ix ) + End - unit ( x ) + \\ End - unit ( xi ) + End - unit ( xii )\end{matrix}}{\begin{matrix}[ End - unit ( viii ) + End - unit ( ix ) + End - unit ( x ) + \\ End - unit ( xi ) + End - unit ( xii ) + End - unit ( xiii ) + \\ End - unit ( xiv ) + End - unit ( xv ) + End - unit ( xvi ) + \\ End - unit ( xvii ) + End - unit ( xviii ) ]\end{matrix}}$$

The ratio 2 end-groups is defined as

$$Ratio 2 end - groups = \frac{End - unit ( ix )}{\begin{matrix}[ End - unit ( viii ) + End - unit ( ix ) + End - unit ( x ) + \\ End - unit ( xi ) + End - unit ( xii ) + End - unit ( xiii ) + \\ End - unit ( xiv ) + End - unit ( xv ) + End - unit ( xvi ) + \\ End - unit ( xvii ) + End - unit ( xviii ) ]\end{matrix}}$$

The ratio 3 end-groups is defined as

$$Ratio 3 end - groups = \frac{End - unit ( viii )}{\begin{matrix}[ End - unit ( viii ) + End - unit ( ix ) + End - unit ( x ) + \\ End - unit ( xi ) + End - unit ( xii ) + End - unit ( xiii ) + \\ End - unit ( xiv ) + End - unit ( xv ) + End - unit ( xvi ) + \\ End - unit ( xvii ) + End - unit ( xviii ) ]\end{matrix}}$$

The ratio 4 end-groups is defined as

$$Ratio 4 end - groups = \frac{End - unit ( x )}{\begin{matrix}[ End - unit ( viii ) + End - unit ( ix ) + End - unit ( x ) + \\ End - unit ( xi ) + End - unit ( xii ) + End - unit ( xiii ) + \\ End - unit ( xiv ) + End - unit ( xv ) + End - unit ( xvi ) + \\ End - unit ( xvii ) + End - unit ( xviii ) ]\end{matrix}}$$

### Process for Preparing Phenolic Compound Compositions

Phenolic compound compositions (lignin oils) according to the present invention are obtained by a preparation process comprising the following step: subjecting a mixture of (A) a feedstock of lignocellulosic material in a feedstock medium comprising an alcohol or alcohol/water mixture and (B) a catalytic medium comprising an alcohol or alcohol/water mixture, hydrogen gas and a catalyst to a temperature of at least 150° C. This may be embodied as (i) subjecting lignocellulose, lignocellulosic material or a feedstock comprising lignocellulose in a medium of alcohol or alcohol/water mixture to a temperature of at least 150° C. and (ii) separately) subjecting to a temperature of at least 150° C. a medium comprising a metal catalyst in an alcohol or alcohol/water mixture under a hydrogen atmosphere and iii) supplying the reaction product of the processed lignocellulosic material to the catalyst medium. The catalytic medium is preferably pressurized under hydrogen gas. The catalytic medium may receive the hydrogen gas from an external source. An example of suitable catalysts are catalysts comprising ruthenium and/or nickel and/or palladium and/or other transition metals such as cupper, platinum, iridium, rhodium, cobalt, iron, osmium and the like.

In a particular embodiment, the feedstock medium and the catalytic medium are in separate vessels. In yet another particular embodiment the feedstock medium and the catalytic medium are in the same vessel. In another embodiment the reaction vessel is pressurized under hydrogen gas or the reaction vessels are pressurized under the hydrogen gas. By using the inventive method described above it is possible to produce the mixture of phenolic compounds.

In a preferred embodiment of some of the methods described above, the externally supplied hydrogen gas is at a partial pressure of 1 bar or higher at room temperature, with the externally supplied hydrogen gas being at a partial pressure of 10 bar or higher at room temperature being particularly preferred and the externally supplied hydrogen gas being at a partial pressure between 10 and 30 bar at room temperature being especially preferred so that:
A) the ratio 1 end-groups is higher than 0.5,
B) and/or the ratio β-5 is higher than 0.1
C) and/or the ratio β-β is higher than 0.25
D) and/or the ratio β-1 is higher than 0.15

In another preferred embodiment of some of the methods described above the contact time at the reaction temperature of the reaction product of the processed lignocellulosic material with the catalytic medium is higher than 0.0001 h with a contact time of 0.05 h or higher being preferred so that:
A) the ratio 1 end-groups is higher than 0.5
B) and/or the ratio β-5 is higher than 0.1
C) and/or the ratio β-β is higher than 0.25
D) and/or the ratio β-1 is higher than 0.15

In a yet another preferred embodiment of some of the methods described above the catalyst comprises palladium so that:
A) the ratio 1 end-groups is higher than 0.8
B) and/or the ratio β-5 is higher than 0.8
C) and/or the ratio β-β is higher than 0.6
D) and/or the ratio β-1 is higher than 0.8
E) and/or the ratio 2 end-groups is higher than 0.7 if sufficient hydrogen (>10 bar) is provided to the reaction medium
F) and/or the ratio 4 end-groups is lower than 0.1

In yet another embodiment of some of the methods described above the catalyst comprises ruthenium so that:
A) the ratio 1 end-groups is higher than 0.7
B) and/or the ratio β-5 is higher than 0.7
C) and/or the ratio β-β is higher than 0.6
D) and/or the ratio β-1 is higher than 0.8
E) and/or the ratio 2 end-groups is lower than 0.25 if an alcohol solvent is used
F) and/or the ratio 2 end-groups is higher than 0.7 if an alcohol/water mixture is used as a solvent
G) and/or the ratio 4 end-groups is higher than 0.4 if an alcohol solvent is used
H) and/or the ratio 4 end-groups is lower than 0.2 if an alcohol/water solvent is used.

In yet another embodiment of some of the methods described above the catalyst comprises nickel so that:
A) the ratio 1 end-groups is higher than 0.5
B) and/or the ratio β-5 is higher than 0.25
C) and/or the ratio β-1 is higher than 0.5
D) and/or the ratio 2 end-groups is higher than 0.35
E) and/or the ratio 4 end-groups is lower than 0.3.

In yet another embodiment of some of the methods described above the alcohol solvent is a mono- or difunctional alcohol such as: methanol, ethanol, n-propanol, 2-propanol, n-butanol, iso-butanol, tert-butanol, 2-butanol, 1-pentanol, 2-pentanol, 3-methylbutano-1-ol, 2-ethyl hexan-1-ol, ethylene glycol, propylene glycol or a mixture thereof.

In yet another embodiment of some of the methods described above the alcohol/water solvent is mixture of water and a a mono- or difunctional alcohol solvent such as: methanol, ethanol, n-propanol, 2-propanol, n-butanol, iso-butanol, tert-butanol, 2-butanol, 1-pentanol, 2-pentanol, 3-methylbutano-1-ol, 2-ethyl hexan-1-ol, ethylene glycol, propylene glycol or a mixture thereof.

In yet another embodiment of some of the methods described above the mass of the resulting phenolic compounds is between 90 and 10000 g/mol.

In yet another embodiment of some of the methods described above the dispersity index of the resulting phenolic compounds is lower than 2.5, with a dispersity index lower than 2 being preferred.

In yet another embodiment of some of the methods described above the catalyst comprises palladium so that:
A) The ratio 1 end-groups is higher than 0.8 due to the selective solvolytic and hydrogenolytic cleavage of the β-O-4 linkage and the selective hydrogenation to the resulting 4-propanol end-unit in the oligomeric products, if sufficient hydrogen is present, so that the ratio 2 end-groups is higher than 0.2
B) The ratio β-5 is higher than 0.8 due to the selective solvolytic and hydrogenolytic cleavage of the β-5 phenylcoumaran linkage, whereby the molecular ratio of β-5 γ-OH to β-5 E is lower than 10.
C) The ratio β-β is higher than 0.6 due to the selective solvolytic and hydrogenolytic cleavage of the β-β resinol linkage, whereby the molecular ratio of β-β 2×γ-OH to β-βTHF is higher than 0.1.
D) The ratio β-1 is higher than 0.8 due to the selective solvolytic and hydrogenolytic action, whereby the molecular ratio of β-1 γ-OH to β-1 E is higher than 0.1.
E) The phenolic and aliphatic OH content of the lignin mixture is both higher than 2.5 mmol OH per gram of said lignin mixture.

In a preferred embodiment, the catalyst comprises palladium in the presence of a hydrogen pressure higher than 5 bar so that
A) The ratio 1 end-groups is higher than 0.8 due to the selective solvolytic and hydrogenolytic cleavage of the β-O-4 linkage and the selective hydrogenation to the resulting 4-propanol end-unit in the oligomeric products, if sufficient hydrogen is present, so that the ratio 2 end-groups is higher than 0.7
B) The ratio β-5 is higher than 0.8 due to the selective solvolytic and hydrogenolytic cleavage of the β-5 phenylcoumaran linkage, whereby the molecular ratio of β-5 γ-OH to β-5 E is lower than 5.
C) The ratio β-β is higher than 0.6 due to the selective solvolytic and hydrogenolytic cleavage of the β-β resinol linkage, whereby the molecular ratio of β-β2×γ-OH to β-β THF is higher than 1.
D) The ratio β-1 is higher than 0.8 due to the selective solvolytic and hydrogenolytic action, whereby the molecular ratio of β-1 γ-OH to β-1 E is higher than 0.2.
E) The phenolic OH content of the lignin mixture is higher than 4 mmol OH per gram of said lignin mixture and the aliphatic OH content of the lignin mixture is higher than 3.5 mmol OH per gram of said lignin mixture.

With the catalyst comprising palladium in the presence of a hydrogen pressure higher than 10 bar and the reaction time higher than 0.5 h being particularly preferred so that:
A) The ratio 1 end-groups is higher than 0.9 due to the selective solvolytic and hydrogenolytic cleavage of the β-O-4 linkage and the selective hydrogenation to the resulting 4-propanol end-unit in the oligomeric products, if sufficient hydrogen is present, so that the ratio 2 end-groups is higher than 0.8
B) The ratio β-5 is higher than 0.9 due to the selective solvolytic and hydrogenolytic cleavage of the β-5 phenylcoumaran linkage, whereby the molecular ratio of β-5 γ-OH to β-5 E is lower than 5.
C) The ratio β-β is higher than 0.9 due to the selective solvolytic and hydrogenolytic cleavage of the β-β resinol linkage, whereby the molecular ratio of β-β2×γ-OH to 0-f THF is higher than 1.
D) The ratio β-1 is higher than 0.9 due to the selective solvolytic and hydrogenolytic action, whereby the molecular ratio of β-1 γ-OH to β-1 E is higher than 0.2.
E) The phenolic OH content of the lignin mixture is higher than 4 mmol OH per gram of said lignin mixture and the aliphatic OH content of the lignin mixture is higher than 3.5 mmol OH per gram of said lignin mixture.

In yet another embodiment of some of the methods described above the catalyst comprises ruthenium so that:
A) The ratio 1 end-groups is higher than 0.7 due to the selective solvolytic and hydrogenolytic cleavage of the β-O-4 linkage and the selective hydrogenolysis and hydrogenation to the resulting 4-propyl end-unit, if sufficient hydrogen is present, so that the ratio 4 end-groups is higher than 0.2
B) The ratio β-5 is higher than 0.7 due to the selective solvolytic and hydrogenolytic cleavage of the β-5 phenylcoumaran linkage, whereby the molecular ratio of β-5 γ-OH to β-5 E is lower than 10.
C) The ratio β-β is higher than 0.6 due to the selective solvolytic and hydrogenolytic cleavage of the β-β resinol linkage, whereby the molecular ratio of β-β2×γ-OH to β-βTHF is higher than 0.1.
D) The ratio β-1 is higher than 0.8 due to the selective solvolytic and hydrogenolytic action, whereby the molecular ratio of β-1 γ-OH to β-1 E is higher than 0.1.
E) The phenolic OH content of the lignin mixture is higher than 3 mmol OH per gram of said lignin mixture and the aliphatic OH content of the lignin mixture is higher than 1 mmol OH per gram of said lignin mixture.

In a preferred embodiment, the catalyst comprises ruthenium in the presence of a hydrogen pressure higher than 5 bar so that
A) The ratio 1 end-groups is higher than 0.8 due to the selective solvolytic and hydrogenolytic cleavage of the β-O-4 linkage and the selective hydrogenation to the resulting 4-propyl end-unit, if sufficient hydrogen is present, so that the ratio 4 end-groups is higher than 0.5
B) The ratio β-5 is higher than 0.8 due to the selective solvolytic and hydrogenolytic cleavage of the β-5 phenylcoumaran linkage, whereby the molecular ratio of β-5 γ-OH to β-5 E is lower than 5.
C) The ratio β-β is higher than 0.6 due to the selective solvolytic and hydrogenolytic cleavage of the β-β resinol linkage, whereby the molecular ratio of 0-2×γ-OH to 0-f THF is higher than 1.
D) The ratio β-1 is higher than 0.8 due to the selective solvolytic and hydrogenolytic action, whereby the molecular ratio of β-1 γ-OH to β-1 E is higher than 0.2.
E) The phenolic OH content of the lignin mixture is higher than 3.5 mmol OH pr gram of said lignin mixture and the aliphatic OH content of the lignin mixture is higher than 1.5 mmol OH per gram of said lignin mixture.

In another preferred embodiment, the catalyst comprises ruthenium in the presence of a hydrogen pressure higher than 10 bar and the reaction time is higher than 0.5 h so that:
A) The ratio 1 end-groups is higher than 0.8 due to the selective solvolytic and hydrogenolytic cleavage of the β-O-4 linkage and the selective hydrogenation to the resulting 4-propyl end-unit, if sufficient hydrogen is present, so that the ratio 4 end-groups is higher than 0.5
B) The ratio β-5 is higher than 0.8 due to the selective solvolytic and hydrogenolytic cleavage of the β-5 phenylcoumaran linkage, whereby the molecular ratio of β-5 γ-OH to β-5 E is lower than 5.
C) The ratio β-β is higher than 0.7 due to the selective solvolytic and hydrogenolytic cleavage of the β-β resinol linkage, whereby the molecular ratio of β-β2×γ-OH to β-βTHF is higher than 1.
D) The ratio β-1 is higher than 0.9 due to the selective solvolytic and hydrogenolytic action, whereby the molecular ratio of β-1 γ-OH to β-1 E is higher than 0.2.
E) The phenolic OH content of the lignin mixture is higher than 3.5 mmol OH program of said lignin mixture and the aliphatic OH content of the lignin mixture is higher than 1.5 mmol OH per gram of said lignin mixture.

In yet another preferred embodiment, the catalyst comprises ruthenium in the presence of a hydrogen pressure higher than 5 bar and an alcohol/water solvent so that:
A) The ratio 1 end-groups is higher than 0.8 due to the selective solvolytic and hydrogenolytic cleavage of the β-O-4 linkage and the selective hydrogenation to the resulting 4-propanol end-unit, if sufficient hydrogen is present, so that the ratio 2 end-groups is higher than 0.5
B) The ratio β-5 is higher than 0.7 due to the selective solvolytic and hydrogenolytic cleavage of the β-5 phenylcoumaran linkage, whereby the molecular ratio of β-5 γ-OH to β-5 E is lower than 10.
C) The ratio β-β is higher than 0.6 due to the selective solvolytic and hydrogenolytic cleavage of the β-β resinol linkage
D) The ratio β-1 is higher than 0.8 due to the selective solvolytic and hydrogenolytic action, whereby the molecular ratio of β-1 γ-OH to β-1 E is higher than 0.2.
E) The phenolic OH content of the lignin mixture is higher than 3.5 mmol OH per gram of said lignin mixture and the aliphatic OH content of the lignin mixture is higher than 3 mmol OH per gram of said lignin mixture.

In yet another preferred embodiment, the catalyst comprises ruthenium in the presence of a hydrogen pressure higher than 5 bar, an alcohol/water solvent and a reaction time higher than 0.5 h so that:
A) The ratio 1 end-groups is higher than 0.9 due to the selective solvolytic and hydrogenolytic cleavage of the β-O-4 linkage and the selective hydrogenation to the resulting 4-propanol end-unit, if sufficient hydrogen is present, so that the ratio 2 end-groups is higher than 0.6
B) The ratio β-5 is higher than 0.7 due to the selective solvolytic and hydrogenolytic cleavage of the β-5 phenylcoumaran linkage, whereby the molecular ratio of β-5 γ-OH to β-5 E is lower than 5.
C) The ratio β-β is higher than 0.6 due to the selective solvolytic and hydrogenolytic cleavage of the β-β resinol linkage
D) The ratio β-1 is higher than 0.9 due to the selective solvolytic and hydrogenolytic action, whereby the molecular ratio of β-1 γ-OH to β-1 E is higher than 0.2.
E) The phenolic OH content of the lignin mixture is higher than 3.5 mmol OH per gram of said lignin mixture and the aliphatic OH content of the lignin mixture is higher than 3 mmol OH per gram of said lignin mixture.

In yet another embodiment of some of the methods described above the catalyst comprises nickel so that:
A) The ratio 1 end-groups is higher than 0.5 due to the selective solvolytic and hydrogenolytic cleavage of the β-O-4 linkage and the selective hydrogenation to the resulting 4-propanol end-unit, if sufficient hydrogen is present, so that the ratio 2 end-groups is higher than 0.2
B) The ratio β-5 is higher than 0.25, whereby the molecular ratio of β-5 γ-OH to β-5 E is lower than 10.
C) The ratio β-1 is higher than 0.5 due to the selective solvolytic and hydrogenolytic action, whereby the molecular ratio of 3-1 γ-OH to β-1 E is lower than 10
D) The phenolic OH and aliphatic OH content of the lignin mixture are both higher than 2.5 mmol OH per gram of said lignin mixture

In a preferred embodiment, the catalyst comprises nickel in the presence of a hydrogen pressure higher than 5 bar so that:
A) The ratio 1 end-groups is higher than 0.7 due to the selective solvolytic and hydrogenolytic cleavage of the β-O-4 linkage and the selective hydrogenation to the resulting 4-propanol end-unit, if sufficient hydrogen is present, so that the ratio 2 end-groups is higher than 0.5
B) The ratio β-5 is higher than 0.5 due to the selective solvolytic and hydrogenolytic cleavage of the β-5 phenylcoumaran linkage, whereby the molecular ratio of β-5 γ-OH to β-5 E is lower than 2.
C) The ratio β-1 is higher than 0.5 due to the selective solvolytic and hydrogenolytic action, whereby the molecular ratio of β-1 γ-OH to β-1 E is lower than 2.
D) The phenolic OH and aliphatic OH content of the lignin mixture are both higher than 3 mmol OH per gram of said lignin mixture

In yet another preferred embodiment, the catalyst comprises nickel in the presence of a hydrogen pressure higher than 10 bar, the reaction time is higher than 0.5 h and the feedstock is a softwood so that:
A) The ratio 1 end-groups is higher than 0.7 due to the selective solvolytic and hydrogenolytic cleavage of the β-O-4 linkage and the selective hydrogenation to the resulting 4-propanol end-unit, if sufficient hydrogen is present, so that the ratio 2 end-groups is higher than 0.5
B) The ratio β-5 is higher than 0.5 due to the selective solvolytic and hydrogenolytic cleavage of the β-5 phenylcoumaran linkage, whereby the molecular ratio of β-5 γ-OH to β-5 E is lower than 2.
C) The ratio β-1 is higher than 0.5 due to the selective solvolytic and hydrogenolytic action, whereby the molecular ratio of β-1 γ-OH to β-1 E is lower than 2.
D) The phenolic OH and aliphatic OH content of the lignin mixture are both higher than 3 mmol OH per gram of said lignin mixture
E) The ratio β-3 is higher than 0.3 due to the selective solvolytic and hydrogenolytic cleavage of the β-β resinol linkage

According to a sixteenth aspect of the present invention, a catalytic process for producing aromatic compound compositions from lignin biomass is provided by dispersing the biomass with a catalyst in an alcohol or alcohol/water solvent in a pressurisable container, providing a hydrogen gas pressure greater than 1 bar at room temperature in said container and heating said dispersion to at least 150° C. and heating at said temperature for at least 0 minutes, wherein said catalyst comprises at least one metal selected from the group consisting of ruthenium, palladium, nickel, copper, platinum, iridium, rhodium, cobalt, iron and osmium.

According to a preferred embodiment of the sixteenth aspect of the present invention, said hydrogen pressure at room temperature is between 10 and 50 bar, with between 20 and 40 bar being preferred.

According to another preferred embodiment of the sixteenth aspect of the present invention, the biomass/catalyst weight ratio is between 5 and 20.

According to another preferred embodiment of the sixteenth aspect of the present invention, the dispersion is heated at the temperature for between 0 and 180 minutes, with 30 to 180 minutes being preferred.

According to another preferred embodiment of the sixteenth aspect of the present invention, the temperature is between 200 and 250° C.

According to another preferred embodiment of the sixteenth aspect of the present invention, the at least one metal is selected from the group consisting of ruthenium, palladium and nickel.

In certain embodiments, the bioaromatic composition, according to the present invention, has an increased reactivity in comparison to other aromatic compositions derived from lignin or lignocellulose conversion techniques. This increased reactivity is the consequence of the engineered structure and can be expressed by a higher reaction rate or higher reaction rate constant.

In further embodiments, the bioaromatic composition, according to the present invention, is completely soluble in polar organic solvents such as: ethyl acetate, methyl acetate, methanol, ethanol, propanol, isopropanol, butanol, 2-butanol, tert-butanol, tetrahydrofuran, dioxane, gamma-valerolactone, acetone, acetonitrile, dichloromethane, and chloroform.

### Use of the Phenolic Compound Composition

In certain embodiments, the phenolic compoundcomposition, according to the present invention, can be used as:
An additive for resins.
An additive for epoxy-resins.
An additive for phenol-formaldehyde resins.
An intermediary composition in the production of resins.
An intermediary composition in the production of epoxy-resins.
An intermediary composition in the production of lignin-formaldehyde resins.
A starting material in the production of resins.
A starting material in the production of epoxy-resins.
A starting material in the production of lignin-formaldehyde resins.
An additive for polyurethanes.
An additive for flame retardants
An intermediary composition in the production of polyurethanes
An intermediary composition in the production of flame retardants
A starting material in the production of polyurethanes
A starting material in the production of flame retardants.

Such applications enhance and exploit the characteristics of the phenolic compound compositions, according to the present invention. The inherent flame-retardant properties endowed by the presence of covalently bound phosphorus in the phenolic compound compositions, according to the present invention, can be readily enhanced by reacting the aliphatic hydroxy groups present in the phenolic compound compositions with any kind of phosphorus halogen. Reaction of isocyanates with the more numerous aliphatic hydroxy groups present in the phenolic compound compositions, according to the present invention, results in urethane-bond formation opening applications as an additive or intermediary composition in the production of polyurethanes and also more densely crosslinked polyurethanes.

### EXAMPLES

The following examples are put forth so as to provide those of ordinary skill in the art with a complete disclosure and description of how the compounds, articles, devices and/or methods claimed herein are made and evaluated, and are intended to be purely exemplary and are not intended to limit the disclosure. Efforts have been made to ensure accuracy with respect to numbers (e.g. amounts, temperature, etc.), but some errors and deviations should be accounted for.

There are numerous variations and combinations of reaction conditions, e.g., desired solvents, solvent mixtures, temperatures, hydrogen pressures, catalyst combinations, reaction times and other reaction ranges and conditions that can be used to optimize the product selectivity's obtained from the described process. Only reasonable and routine experimentation will be required to optimize such process conditions.

### Example 1 Preparation

All materials and reagents were used as received from the supplier unless otherwise indicated.

The specified amount of lignocellulose material was loaded into a 100 mL stainless steel batch reactor together with the specified amount of catalyst and the specified amount of solvent. Subsequently, the reactor was sealed, flushed three times with N₂ (10 bar) and then pressurized with the specified amount of H₂. Next, the reaction mixture was stirred (600 rpm) and simultaneously heated to the specified temperature. After the specified reaction time, the reactor was cooled and depressurized at room temperature. The reactor contents were quantitatively collected by washing the reactor with acetone.

The solid pulp was separated by filtration and washed thoroughly with acetone. Next, the resulting filtrate was evaporated and a brown oil was obtained, which was subjected to a threefold liquid-liquid extraction using ethylacetate (EtOAc) and water. To obtain the lignin oil, the EtOAc-extracted phase was dried.

Due to a low volatility of lignin dimers and oligomers, the selectivity's of interphenolic linkages can only by measured by NMR. GPC/SEC measurements were performed to confirm the presence of lignin oligomers.

The distribution of the molar mass of the lignin products was investigated using gel permeation chromatography—size exclusion (GPC/SEC). Therefore, a lignin sample was dissolved in THF (5 mg mL⁻¹) and subsequently filtered with a 0.2 μm PTFE membrane to remove any particulate matter to prevent plugging of the column. GPC/SEC analyses were performed at 40° C. on a Waters E2695 equipped with a PL-Gel 3 μm Mixed-E column with at length of 300 mm, using THF as a solvent with a flow of 1 mL min⁻¹. The detection was UV based at a wavelength of 280 nm. Calibration were based on calibration with commercial polystyrene standards of Agilent.

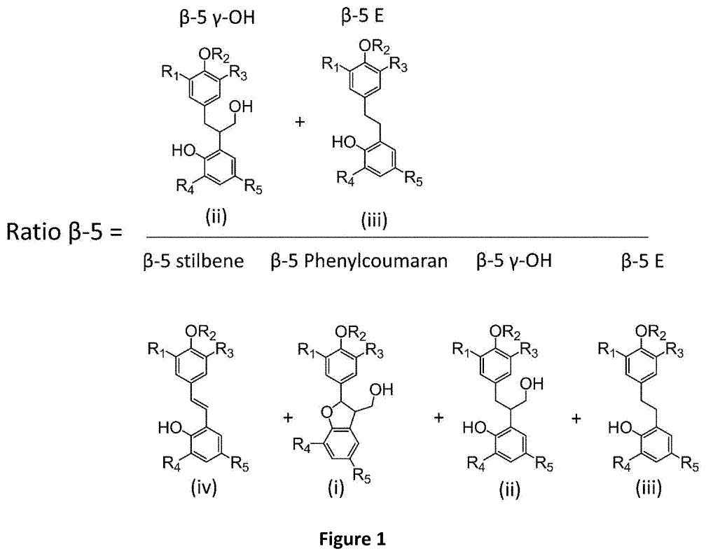

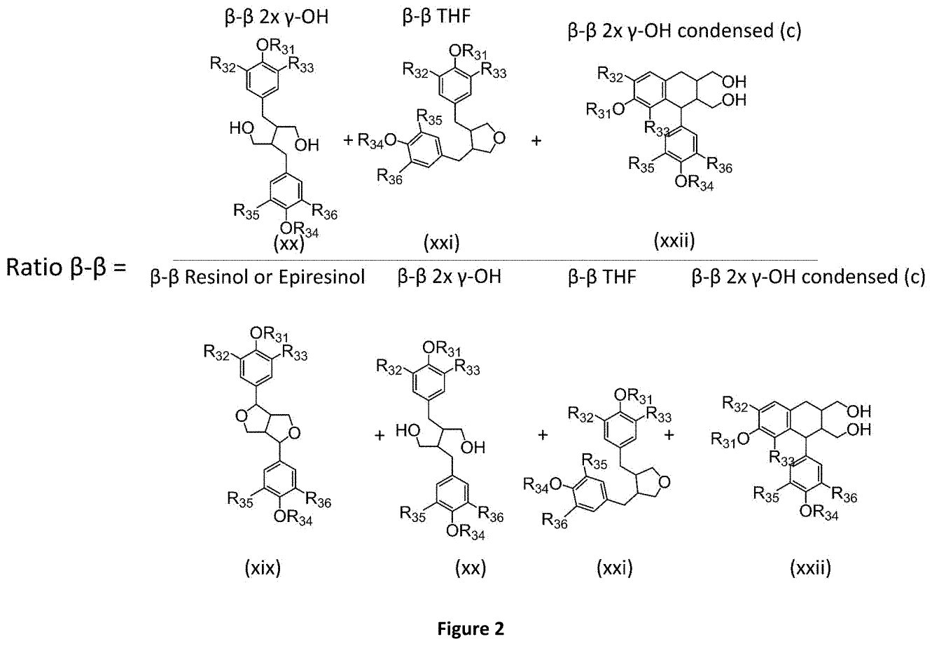

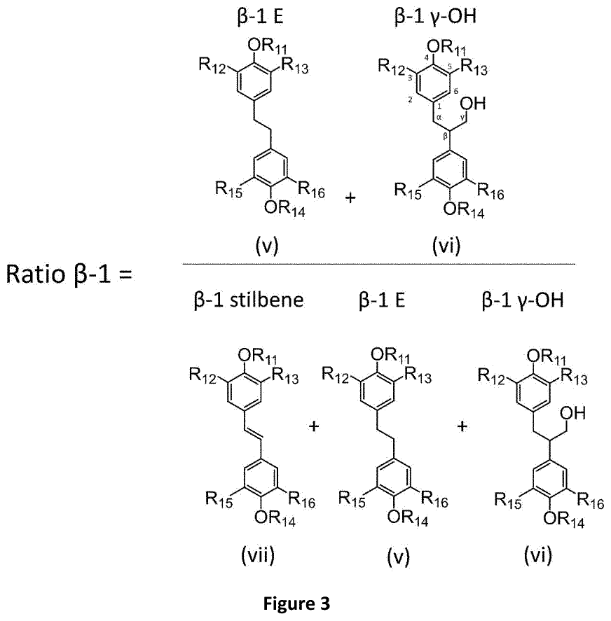

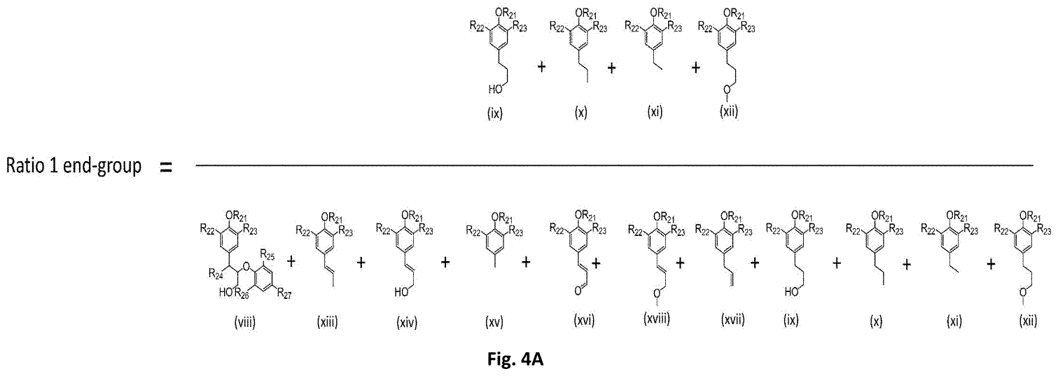

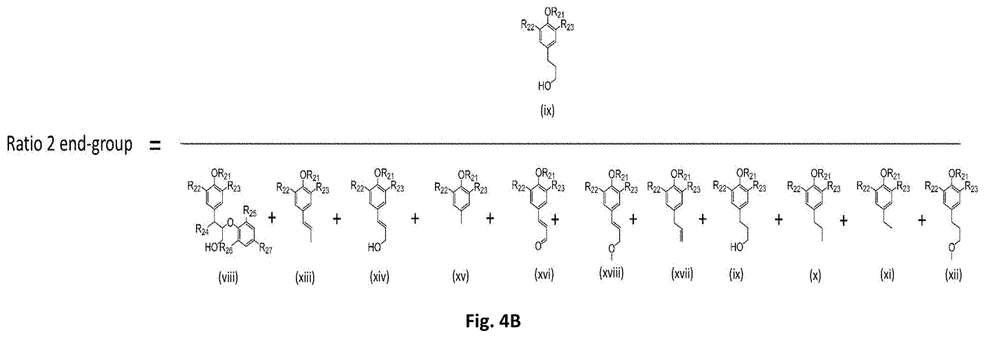

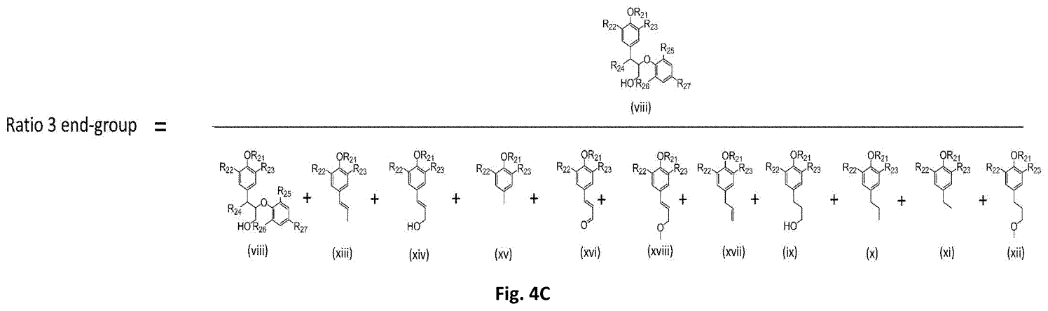

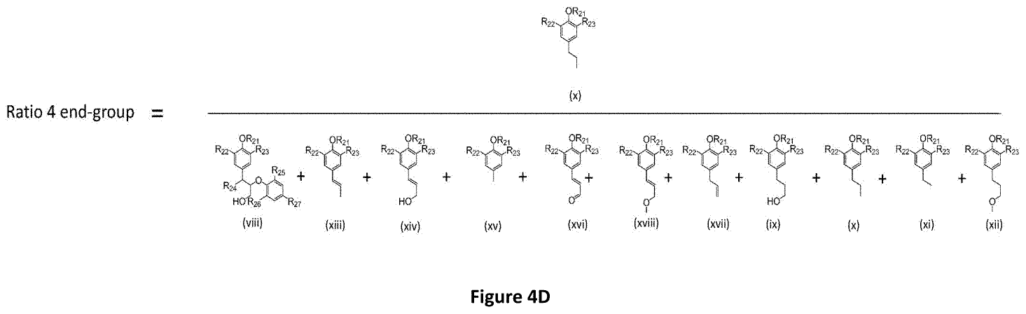

To get insight in the selectivity's of the interphenolic linkages, liquid phase ¹H-¹³C Heteronuclear single quantum coherence spectroscopy (HSQC) NMR was acquired. Approximately 70 mg of the lignin sample was dissolved in 0.6 mL DMSO-d₆ and loaded in an NMR tube. The two-dimensional 1H-¹³C HSQC NMR experiment was conducted at 298K using a Bruker Avance III HD 400 MHz console with a Bruker Ascend™ 400 Magnet, equipped with a 5 mm PABBO probe. A Bruker standard pulse sequence (‘hsqcetgpsp.3’) was used for semi-quantification with the following parameters: spectral width in F2 dimension (¹H) of 13 ppm using 2048 data points, a spectral width in F1 dimension (¹³C) of 165 ppm, using 256 data points, a total of 16 scans were recorded with a 2 s interscan delay (D1). Bruker's Topspin 4.0.2 software was used for data processing and volume integration. The spectra was processed in 2048 data points in the F2 and F1 dimension (with one level of linear prediction and 32 coefficients). The solvent peak of DMSO was used as the internal reference (δ_{C}/δ_{H}: 39.5 ppm/2.49 ppm) following by manually phasing and automatic baseline correction. The volumes with the chemical shifts indicated at Table 1 were integrated to quantitatively obtain information about the selectivity's. These volumetric integrals were divided by an integer factor correcting for the amount of C—H pairs of one chemical shift (Table 1, column ‘Factor’) and used to define the following ratio's.
(1) Ratio for β-5 chemical linkage is as provided in FIG. 1
(2) Ratio for β-β chemical linkage is as provided in FIG. 2
(3) Ratio for Ratio for β-1 chemical linkage is as in FIG. 3
(4) Ratio for end-groups is as in FIG. 4

To quantitatively obtain data on the aliphatic and phenolic hydroxyl content ³¹P-NMR was measured. ³¹P-NMR measurements were performed in triplicate using a standard phosphitylation procedure. A solvent solution (1.6 pyridine: 1 CDCl₃) was used to make stock solutions of the internal standard (cholesterol, 20 mg mL⁻¹) and relaxation agent (chromium acetylacetonate, 10 mg mL⁻¹). An amount of lignin (approximately 20 mg) was accurately weighed and 100 μl of the internal standard solution and 50 μl of the relaxation agent solution was added, next to 400 μl of solvent solution. Subsequently, 75 μl of 2-chloro-4,4,5,5-tetramethyl-,1,3,2-dioxaphospholane (TMDP) was added and the sample was thoroughly mixed before transferring them to the NMR-tube. ³¹P-NMR spectra were obtained on a Bruker Avance III 400 MHz NMR using a standard phosphorous pulse program (inverse gated, 128 scans, 5 s interscan delay, O1P 140 ppm). The chemical shifts were calibrated by assigning the sharp peak of residual water+TMDP at 132.2 ppm and automatic baseline correction was applied.

### COMPARATIVE EXAMPLES (1-6)

The lignin oils of comparative examples (1-6) were obtained using the general procedures of general methods and materials and the specific reaction conditions as specified in Table 2. The resulting lignin oils were analyzed according to the procedure in the general methods and materials.

Comparative examples 1-6 are included to establish base values for the different ratio's (ratio 3-5, ratio β-β, ratio β-1 and ratio (1-4) end-groups) from two different lignocellulose feedstocks without the use of a catalyst.

Ratio β-1 is smaller than 0.15 for all the compositions of the six comparative examples. The main compound in this group of inter-unit linkages is the β-1 stilbene structure, with an abundance >85% in all comparative examples.

Ratio β-5 is 0 for all the compositions of the six comparative examples. Only the non-unique β-5 phenylcoumaran and β-5 stilbene are present in these samples

Ratio β-β- is lower than 0.55 in all the compositions of comparative examples. In the case of a softwood lignocellulose feedstock, the ratio β-β is lower than 0.55, whilst if a hardwood lignocellulose feedstock is used, the ratio β-β is lower than 0.1.

Ratio 1 end-groups is lower than 0.3 in all the compositions of comparative examples.

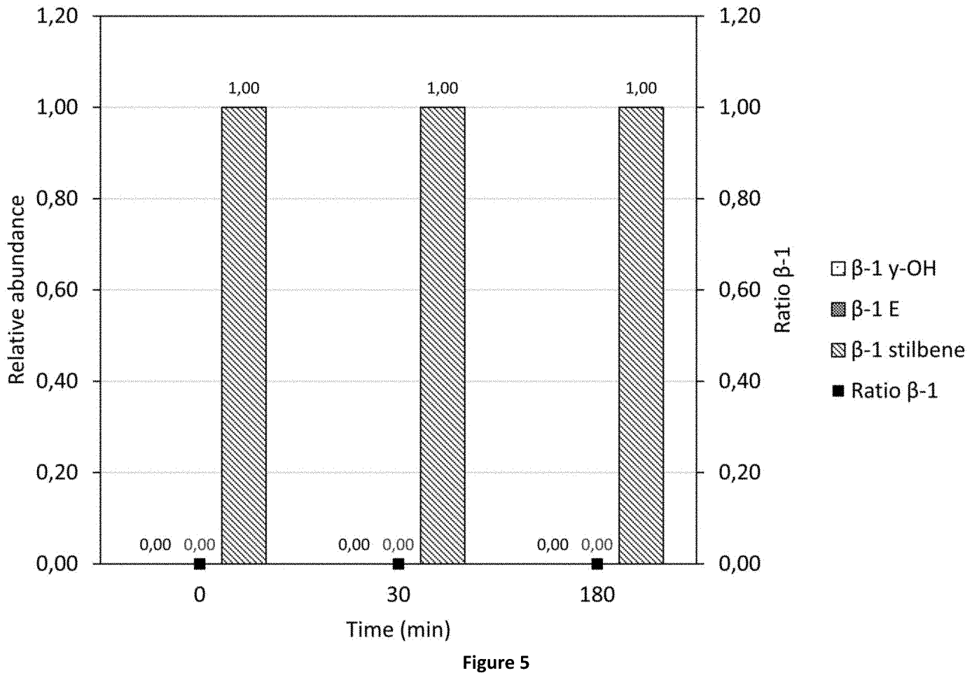

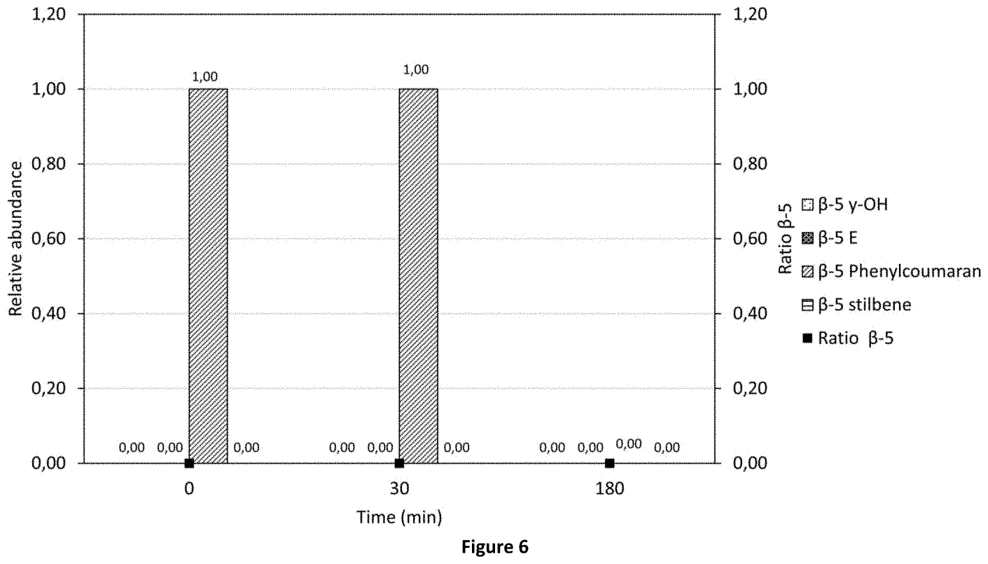

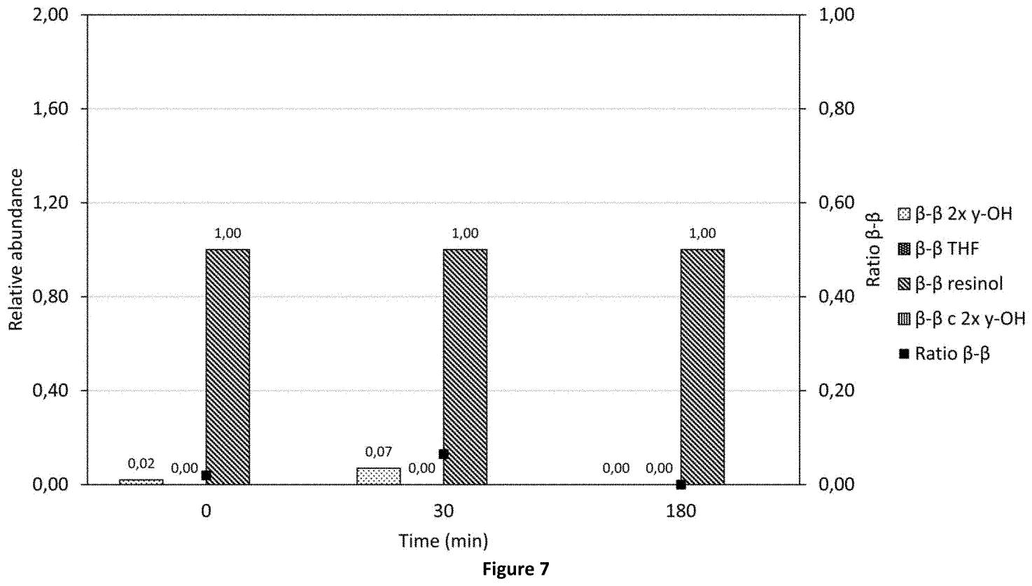

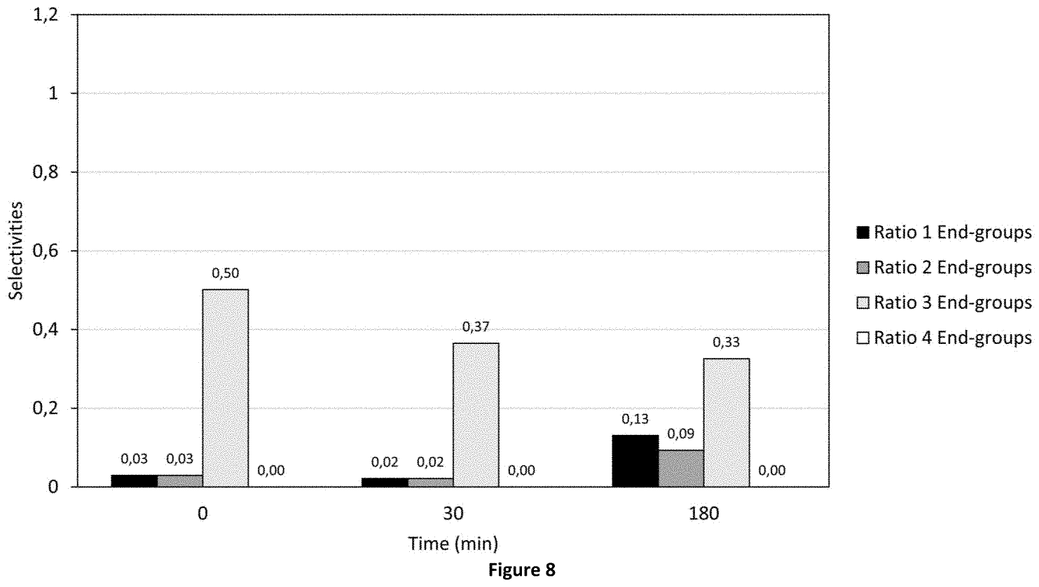

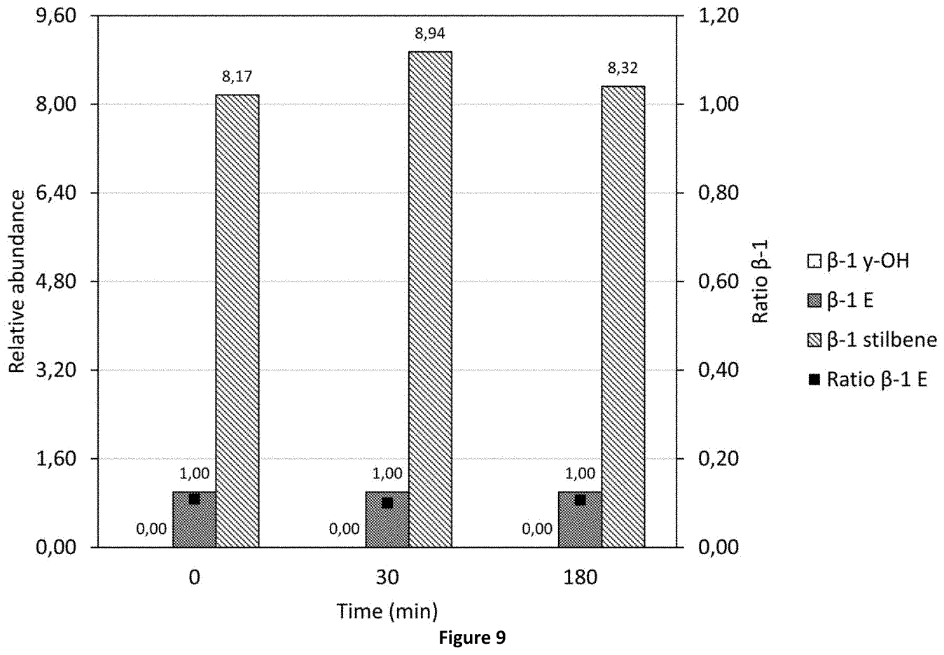

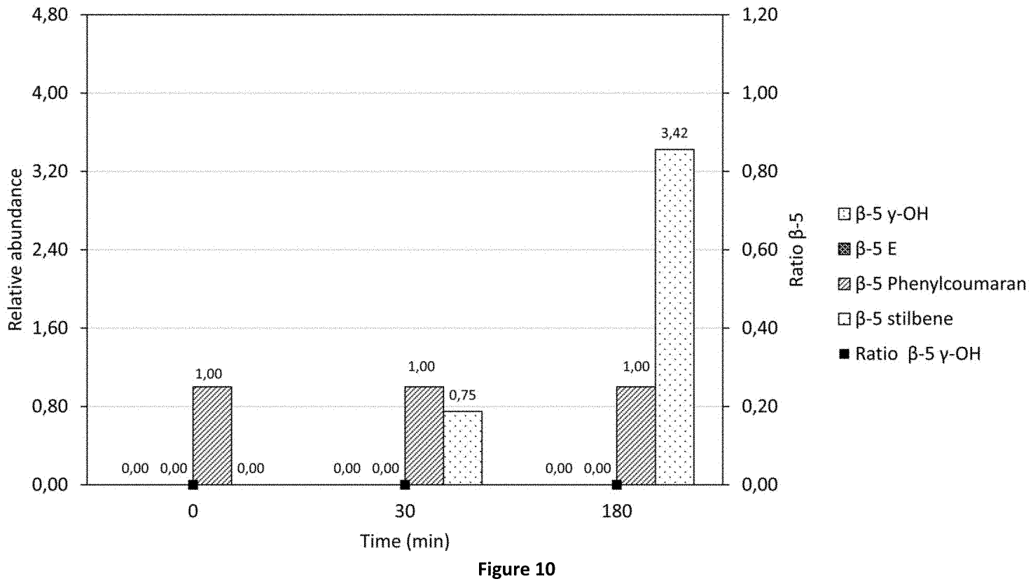

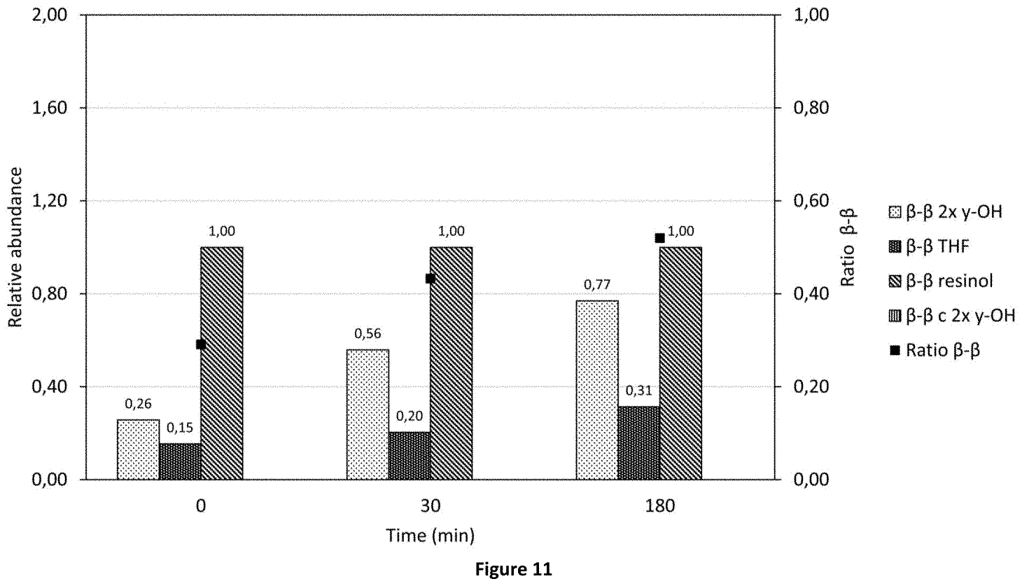

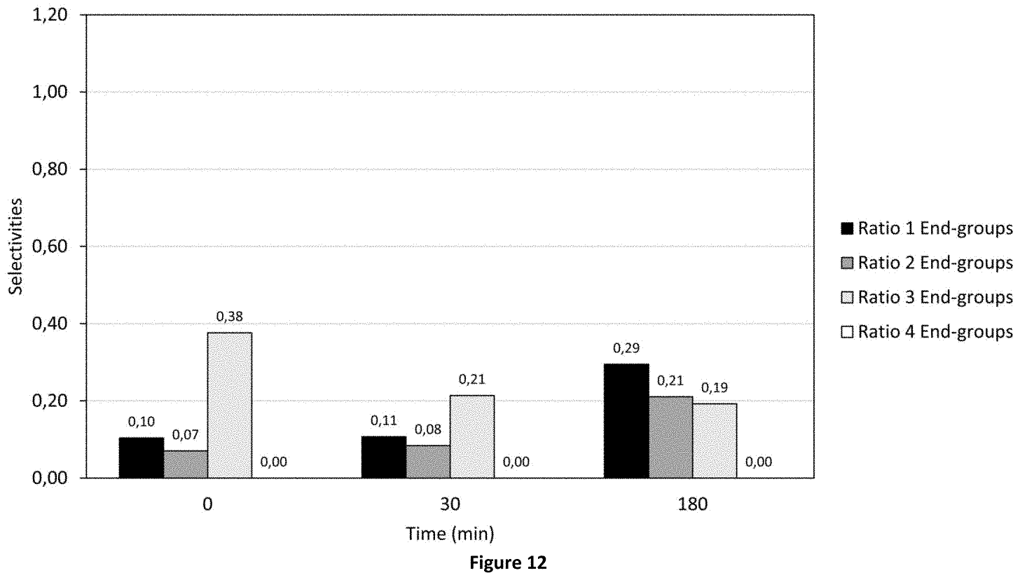

More detailed results on the molecular composition of the different groups are provided in Table 3 and FIGS. 5-12.

### Invention Examples 1-16

The lignin oils of the examples (1-16) were obtained using the general procedures of general methods and materials and the specific reaction conditions as specified in Table 4. The resulting lignin oils were analyzed according to the procedure in the general methods and materials. The examples (1-16), using a Pd/C catalyst on two different types of biomass with varying reaction times, varying reaction temperature and varying hydrogen pressure, are put forth to establish clear similarities in all ratio's (ratio 3-5, ratio β-β, ratio β-1 and ratio (1-4) end-groups), irrespective of the biomass or reaction conditions (temperature, pressure, reaction time). In comparison to the compositions of comparative examples 1-6, all ratio s are higher, showing the clear effect of the catalyst' addition.

Ratio β-1 is equal to 1 for all provided examples (1-16). In comparison to the compositions of comparative examples (1-6), this ratio is clearly higher. This higher ratio is the result of the molecular composition. As shown by examples (1-16), the relative abundances of the unique β-1 E and, β-1 γ-OH structures can be tuned, depending on the desired properties.

Ratio β-5 is higher than 0.8 for all provided examples (1-16). In comparison to the compositions of comparative examples (1-6), this ratio is clearly higher. Moreover, this ratio can be easily increased to 1 as shown. This higher ratio is the result of changes in the molecular composition. As shown by examples (1-16), the relative abundances of the unique β-5 E and, β-5 γ-OH structures can be tuned, depending on the desired properties.

Ratio β-β is higher than 0.5 for all provided examples (1-16). In comparison to the compositions of comparative examples (1-6), the ratio's are clearly higher except for the lignin with entry 6. The ratio β-β can be easily increased to 1 as shown. This higher ratio is the result of changes in the molecular composition. As shown by the compositions of invention examples (1-16), the relative abundances of the unique β-βTHF and, β-β2×γ-OH structures can be tuned, depending on the desired properties.

The ratio 1 end-groups was higher than 0.9 for all the compositions of invention examples 1-16. Furthermore, it is evident that the ratio 2 end-groups is higher than 0.8 if sufficient hydrogen is provided to the catalyst medium. Both ratio 3 end-groups and ratio 4 end-groups are lower than 0.1 in all the compositions of invention examples 1-16.

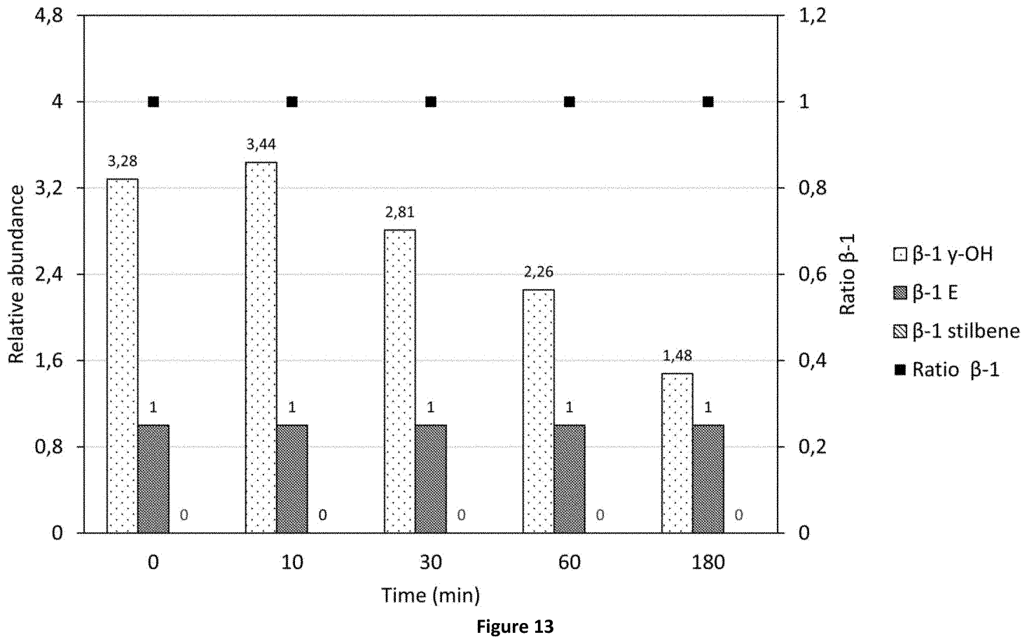

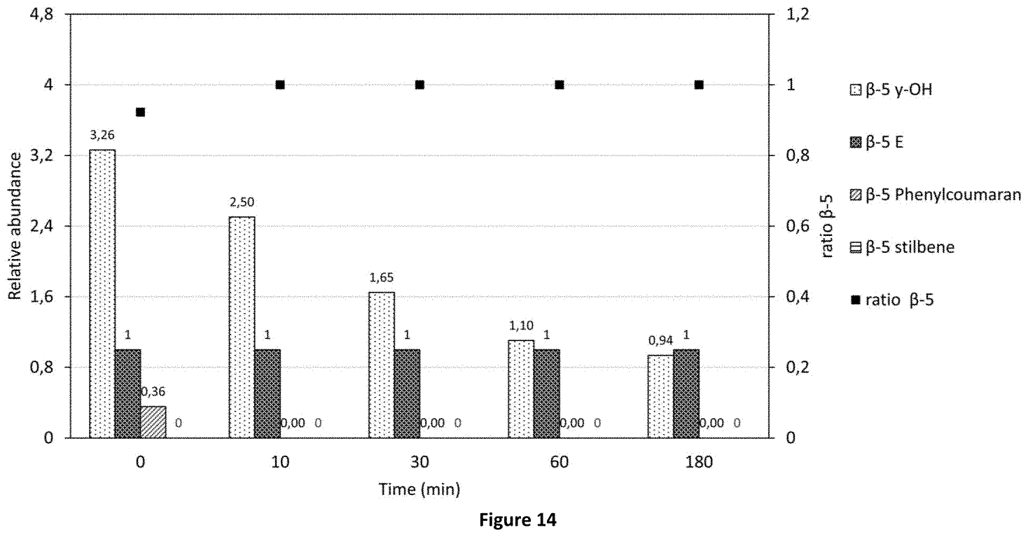

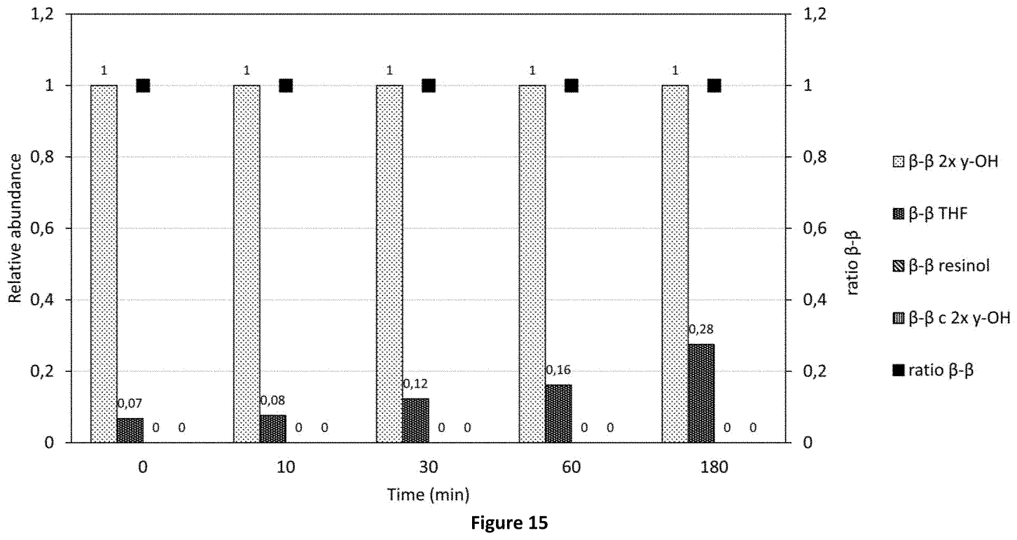

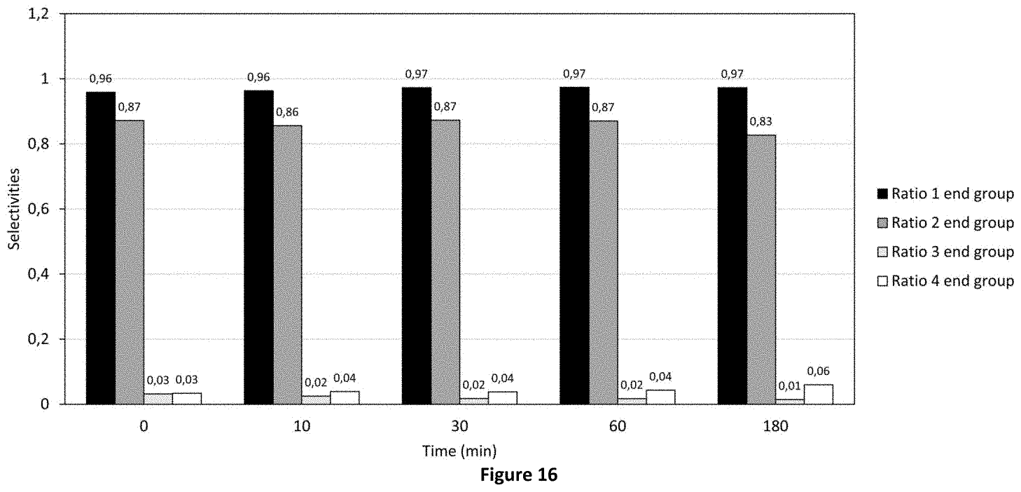

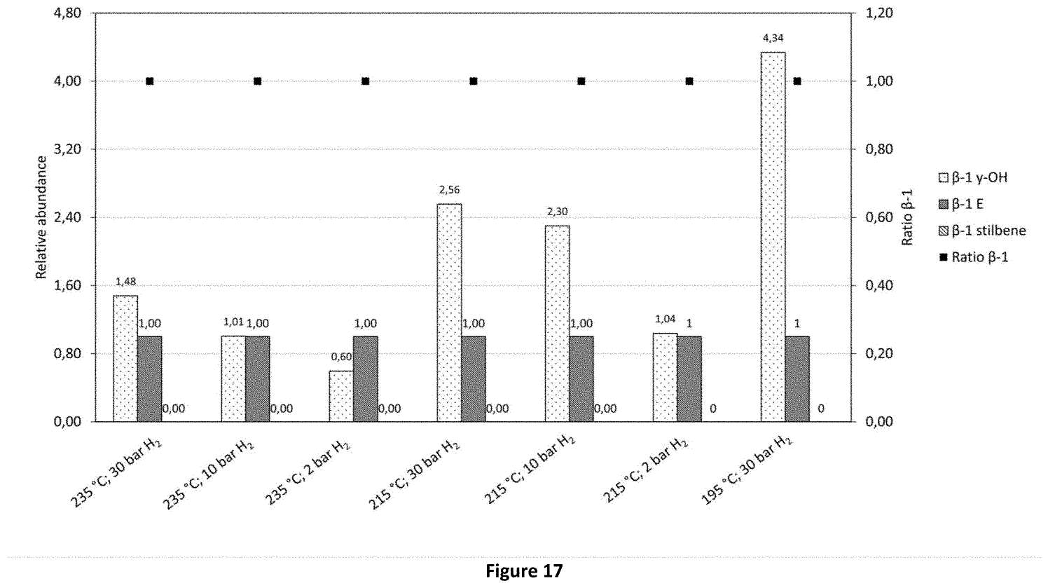

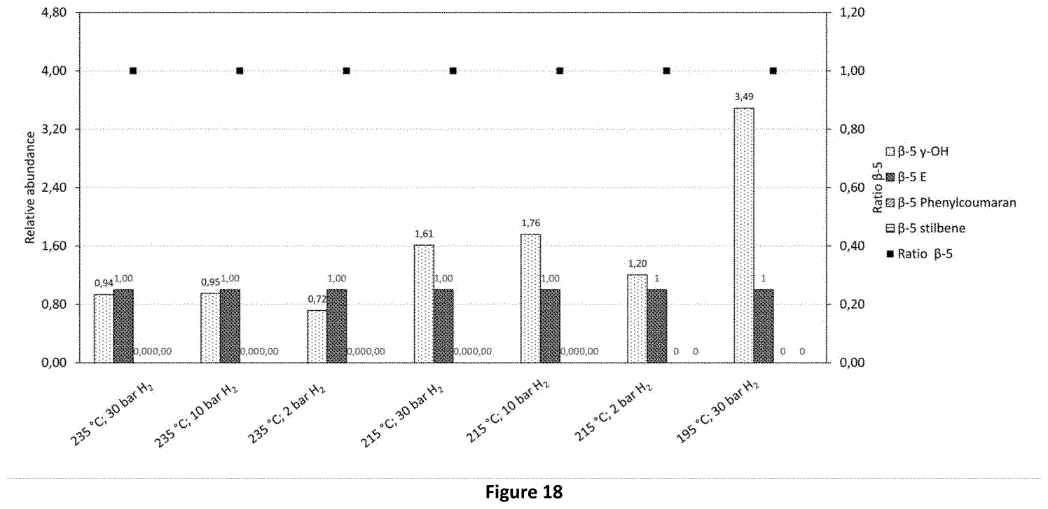

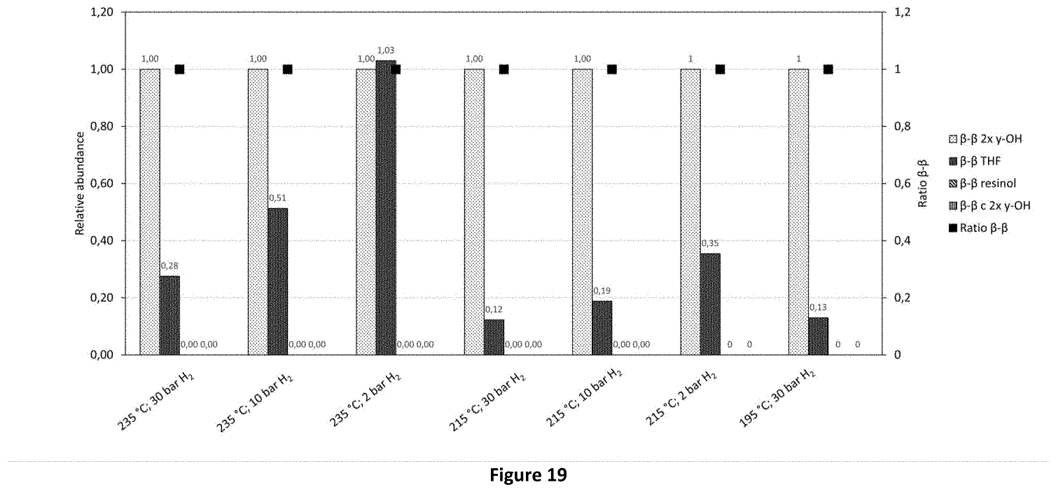

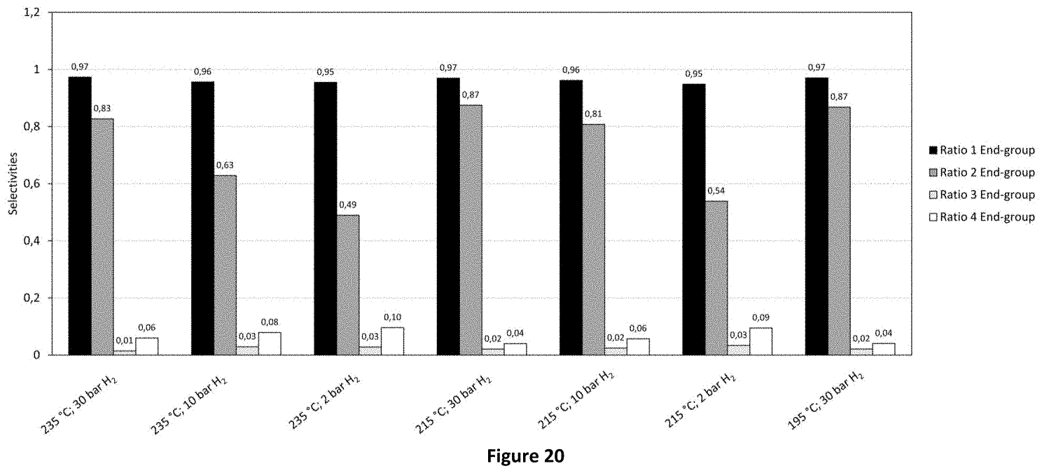

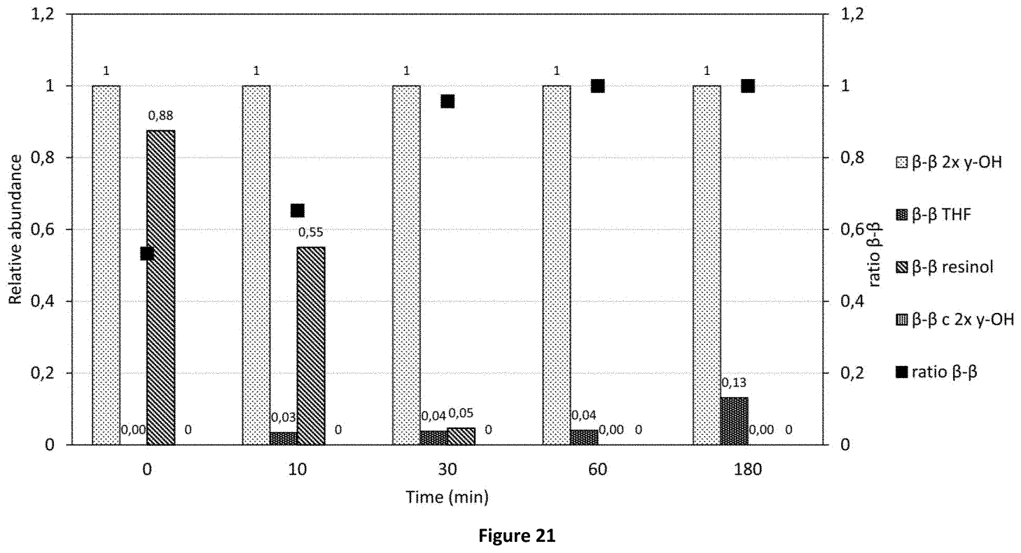

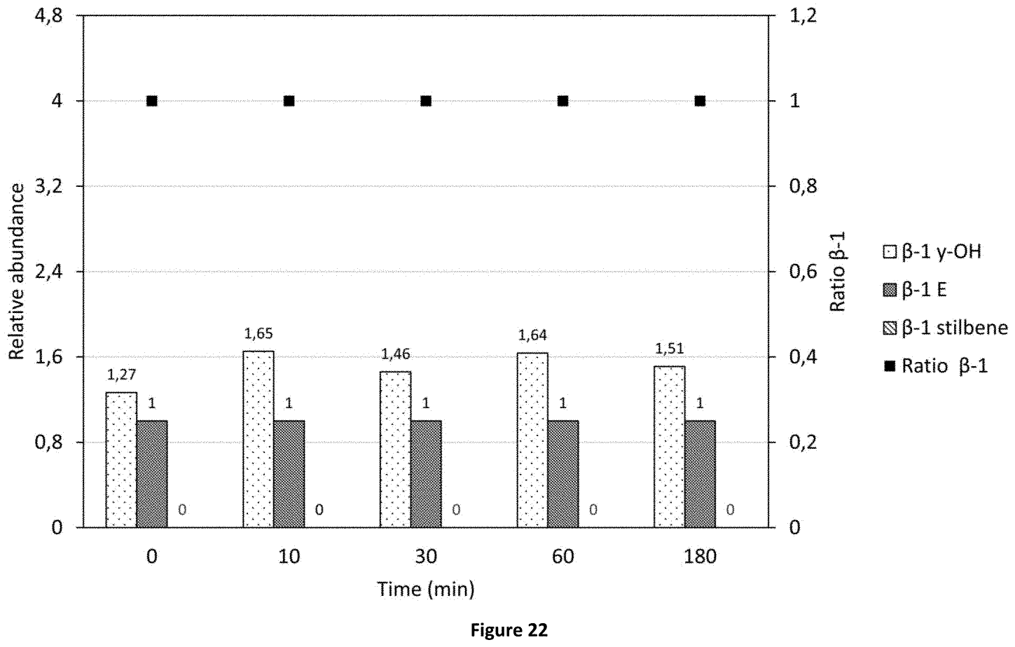

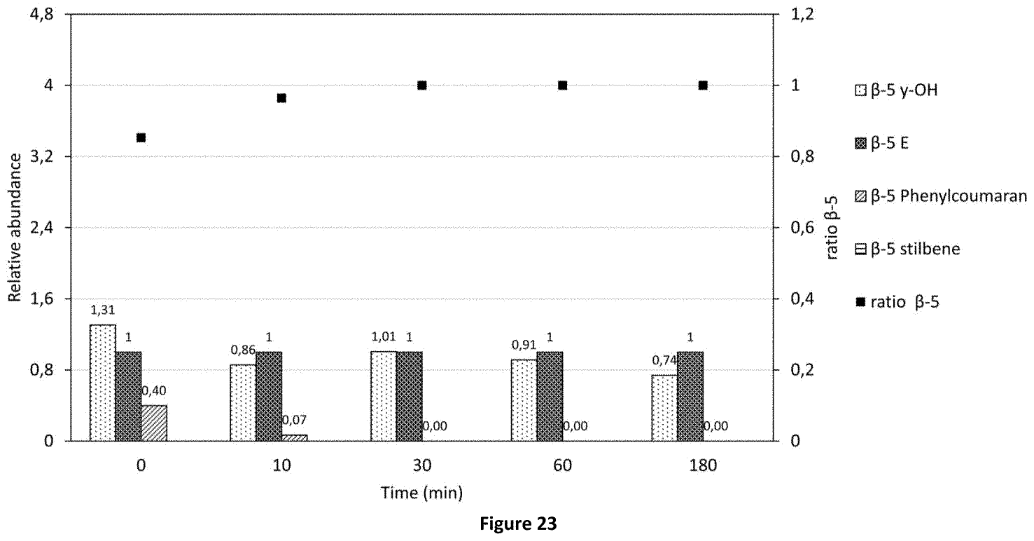

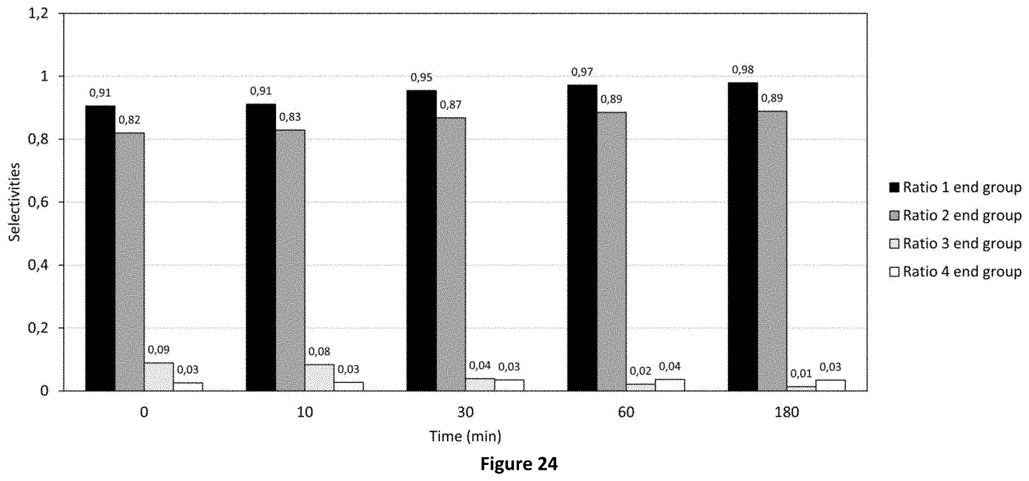

Detailed results on the molecular composition of the different groups are provided in Table 5 and FIGS. 13-24

### Invention Examples 17-30

The lignin oils of the invention examples 17-30 were obtained using the general procedures of general methods and materials and the specific reaction conditions specified in Table 6. The resulting lignin oils were analyzed according to the procedure in the general methods and materials. The compositions of invention examples 17-30 obtained using a Ru/C catalyst on 3 different types of biomass with varying reaction times, varying reaction temperature and varying reaction solvents (methanol and butanol/water (50 volume %/50 volume %)), exhibited clear similarities in ratio's (ratio β-5, ratio β-β, ratio β-1 and ratio 1 end-groups) irrespective of the biomass or reaction conditions (temperature, solvent) and irrespective of the redox catalyst compared with invention examples 1-16. Compared with the compositions of comparative examples 1-6, most ratios were higher, showing the clear effect of catalyst addition.

Ratio β-1 is higher than 0.95 for all invention examples 17-30. In comparison to the comparative examples 1-6, this ratio is clearly higher. This higher ratio is the result of changes in the molecular composition. Invention examples 17-30, show that the relative abundances of the unique β-1 E and, β-1 γ-OH structures can be tuned, depending on the desired properties. Moreover, these relative abundances can be adjusted to obtain similar values to those obtained in the compositions of invention examples 1-16 by adjusting the process conditions, indicating that similar molecular compositions of β-1 are achievable by different metal catalysts.

Ratio β-5 is higher than 0.5 for all the compositions of invention examples 17-30 and higher than 0.7 in the compositions of all the invention examples 17-30 except for invention example 22. In comparison to the compositions of comparative examples 1-6, this ratio is clearly higher. Moreover, this ratio can be easily increased to 1 as illustrated by the compositions of invention examples 22, 26-28 and 30. This higher ratio is the result of differences in the molecular composition. As shown by the compositions of invention examples 17-30, the relative abundances of the unique β-5 E and β-5 γ-OH structures can be tuned, depending on the desired properties. Moreover, these relative abundances can be adjusted to obtain similar values as shown by the compositions of invention examples (1-15) by adjusting the process conditions, indicating that similar molecular compositions of β-5 are achievable by different metal catalysts.

Ratio β-β is higher than 0.25 for all the compositions of the invention examples, except for that of invention example 22. Compared with the compositions of comparative examples 1-6, the ratios are clearly higher. The ratio β-β can be easily increased to 1 as shown. This higher ratio is the result of changes in the molecular composition. As shown by the compositions of invention examples (17-30), the relative abundances of the unique β-βTHF, β-β2×γ-OH c and, β-β2×γ-OH structures can be tuned, depending on the desired properties. Moreover, these relative abundances can be adjusted to obtain similar values as shown by the compositions of invention examples 1-16 by adjusting the process conditions, indicating that similar molecular compositions of β-β are achievable with different metal catalysts. Example 22 is included to show the necessity of prolonged reaction times (more than 0 h) when using Ru/C in combination with hardwoods to obtain the desired ratio β-β.

The ratio 1 end-groups were higher than 0.7 for all the compositions of invention examples 17-30. Furthermore, it is evident that the ratio 2 end-groups was lower than 0.5 if only an alcohol was used as solvent. This is different to the use of Pd as catalyst (compositions of invention examples 1-16) wherein this ratio was higher than 0.8. In contrast, ratio 4 end-groups could be easily increased to values higher than 0.5 or even 0.6 whereas this value was lower than 0.1 if Pd was used as a catalyst. On the other hand, using alcohol/water mixtures as solvent increased the ratio 2 end-groups and ratio 4 end-groups to values similar to those of the compositions of invention examples 1-16.

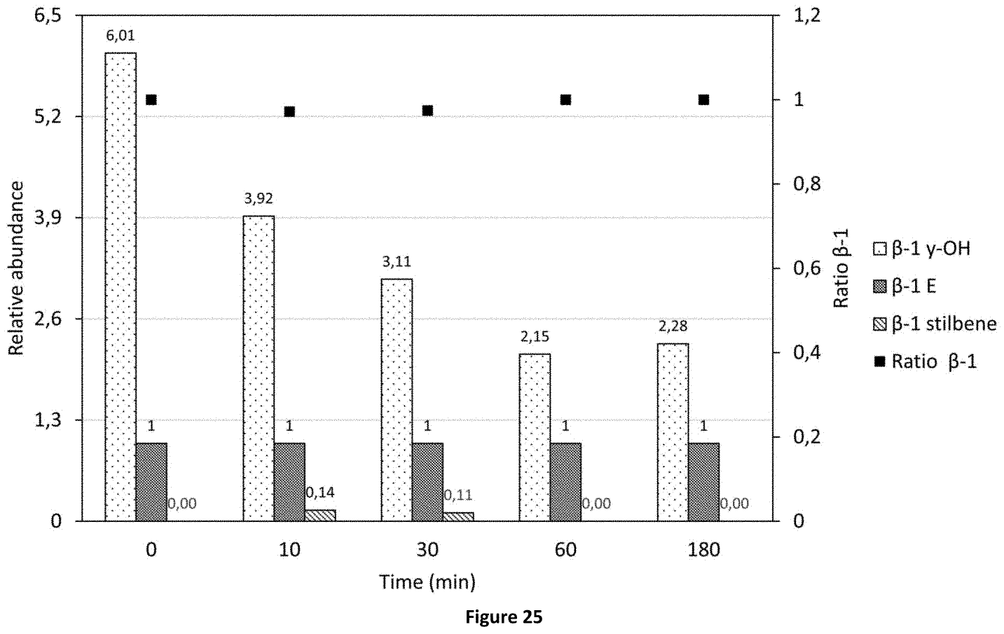

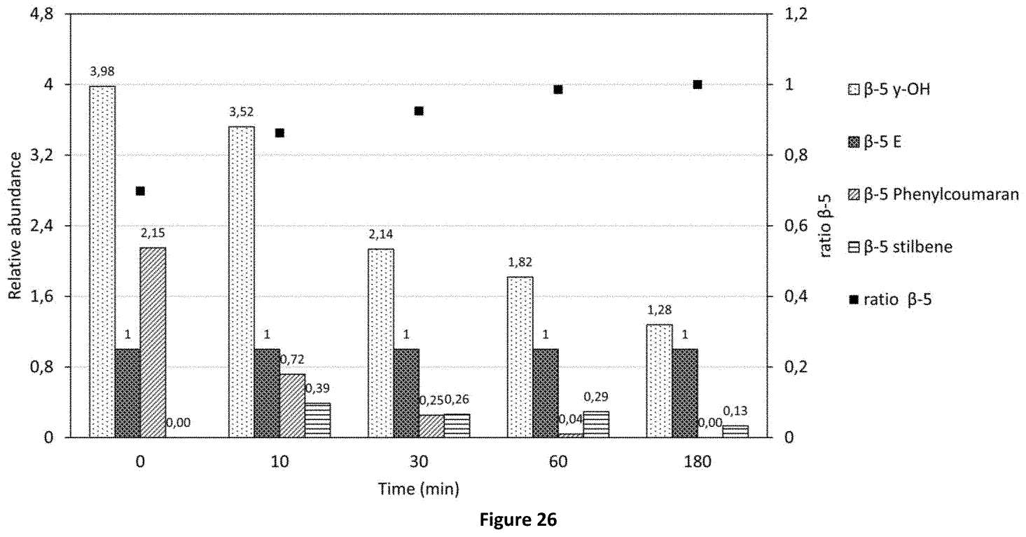

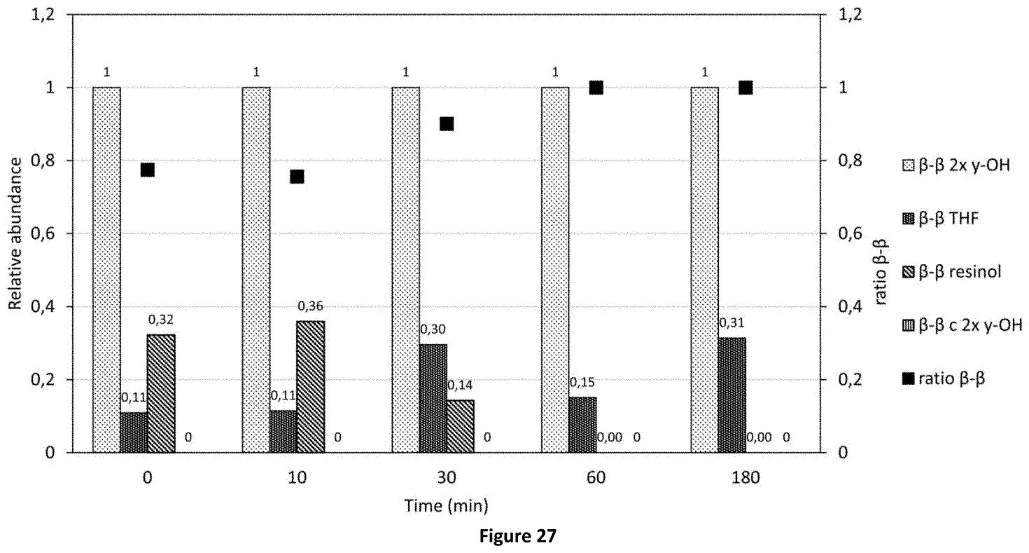

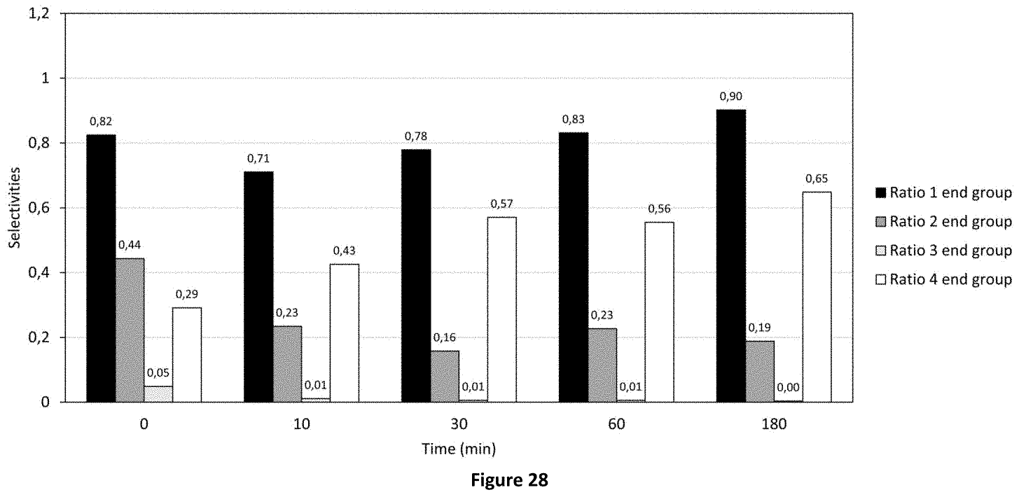

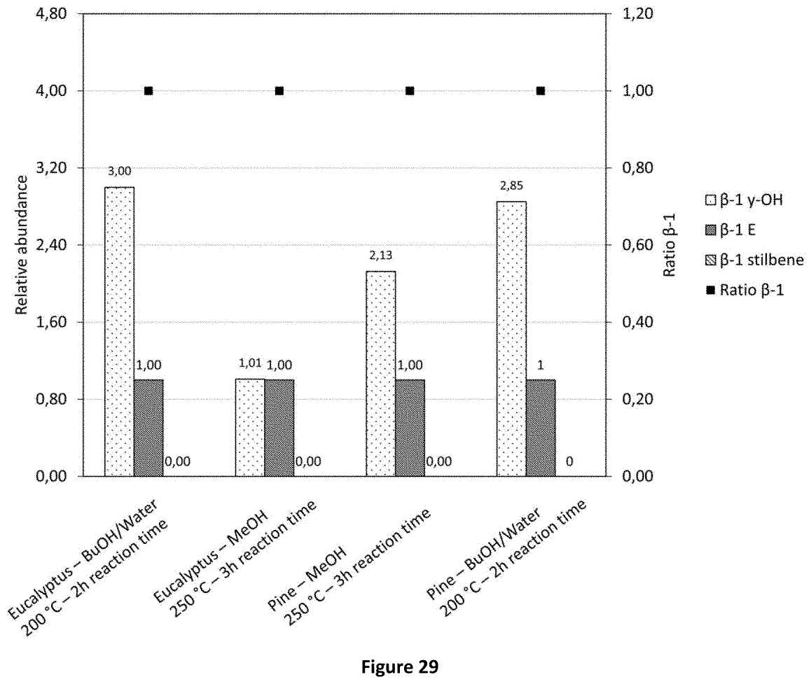

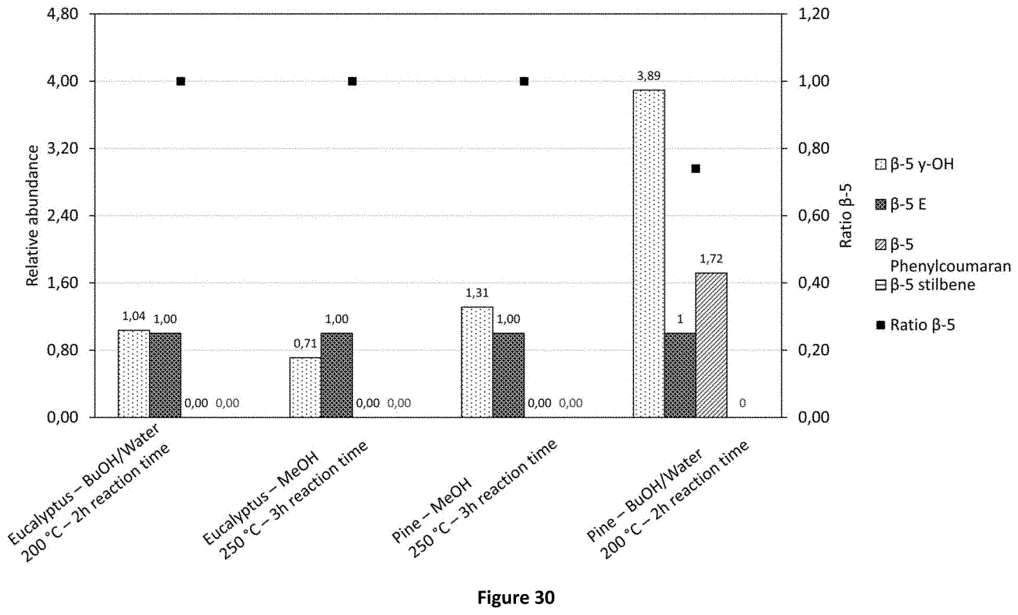

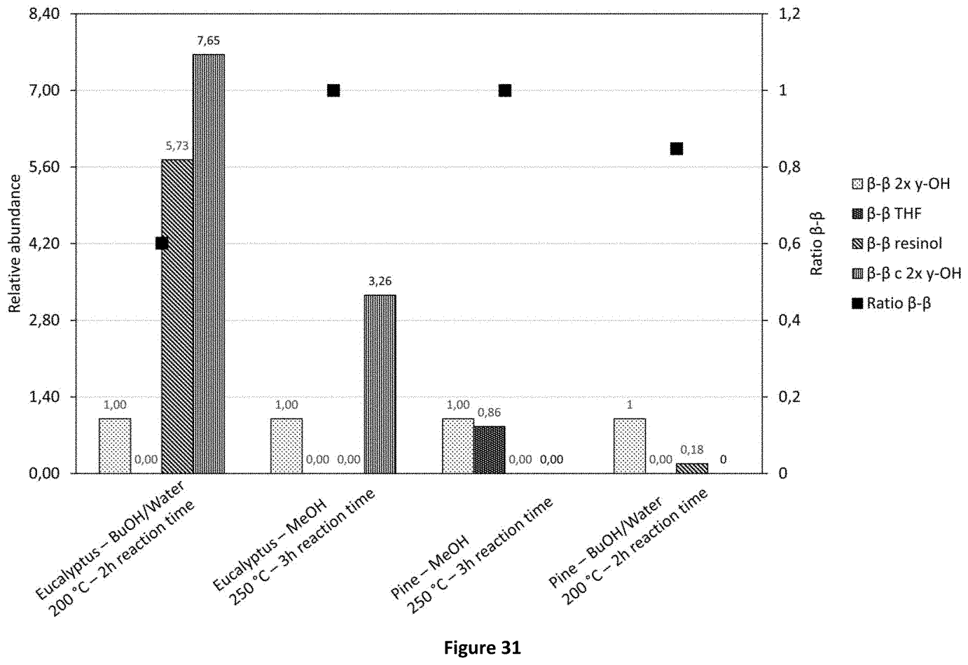

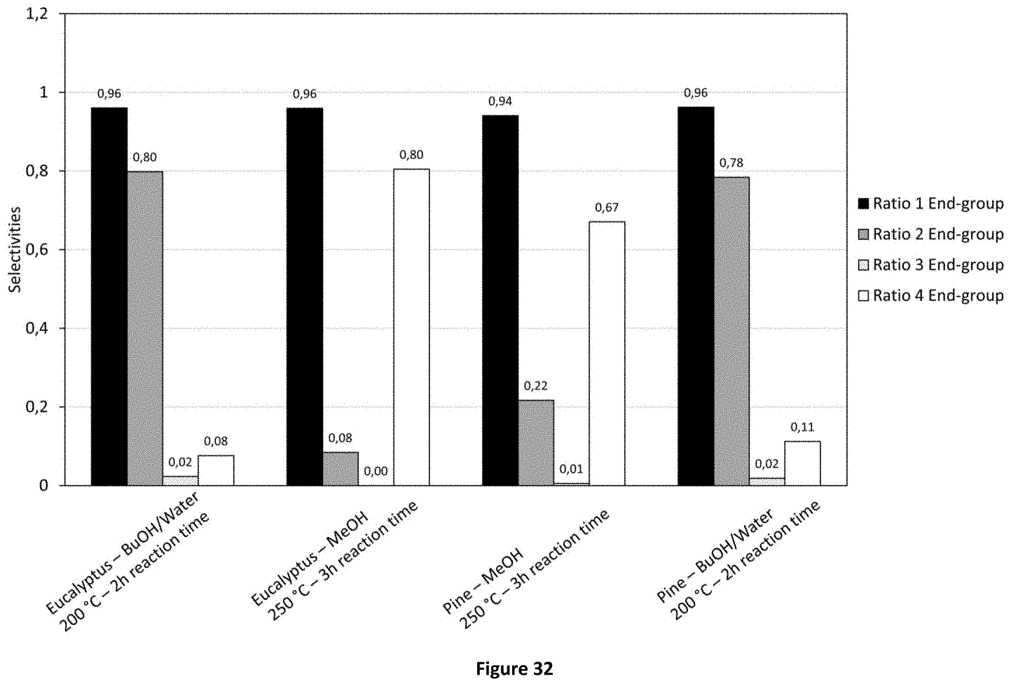

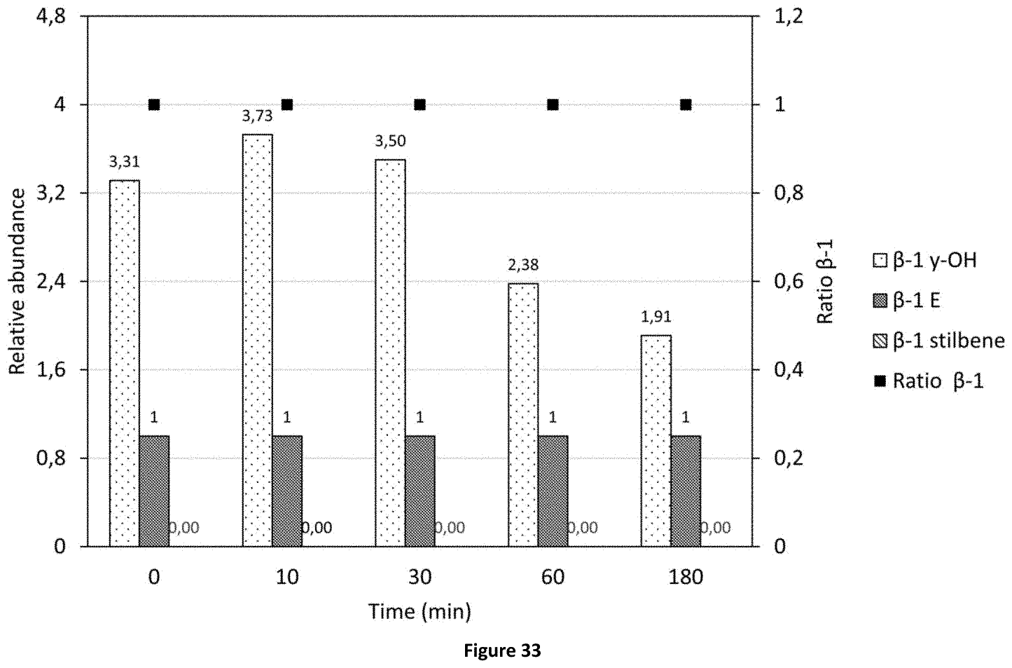

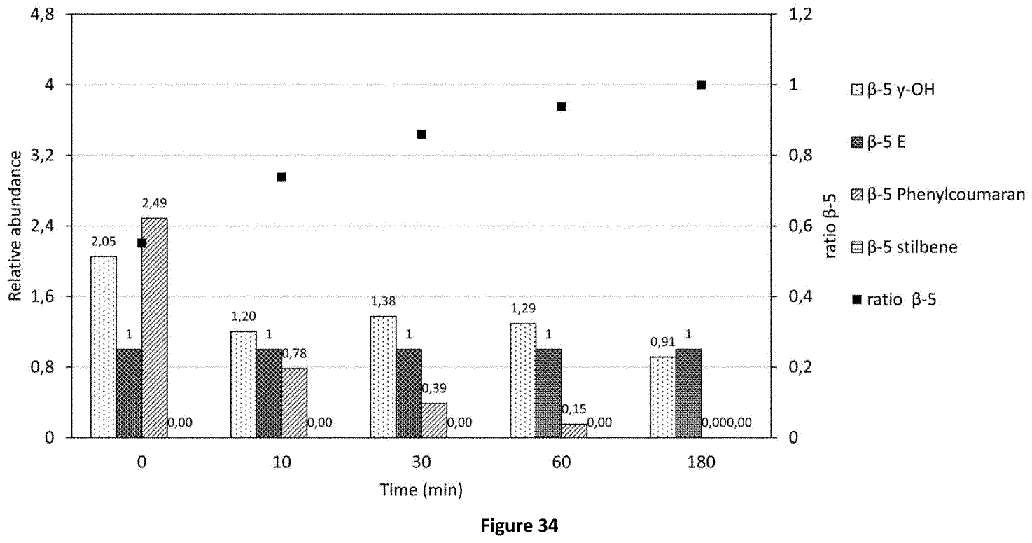

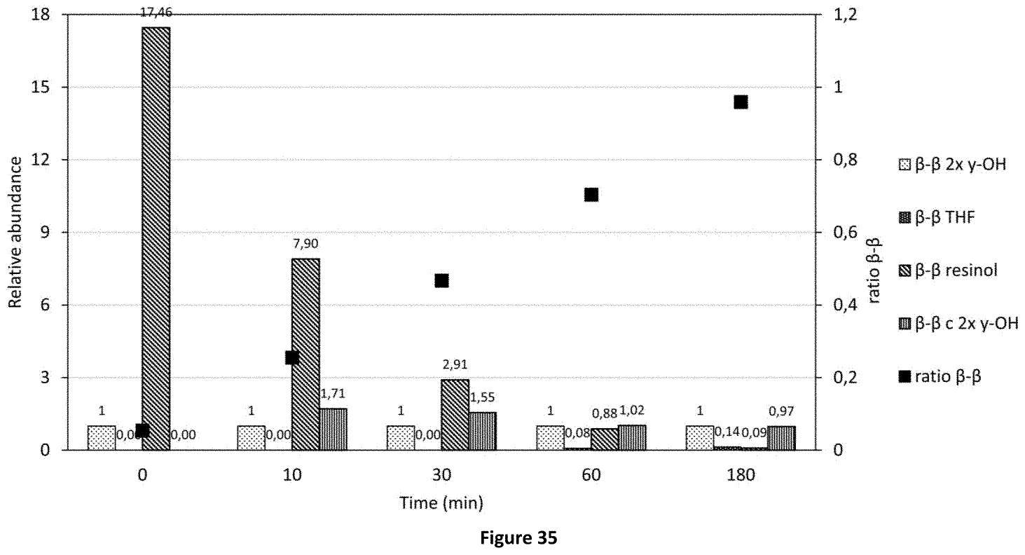

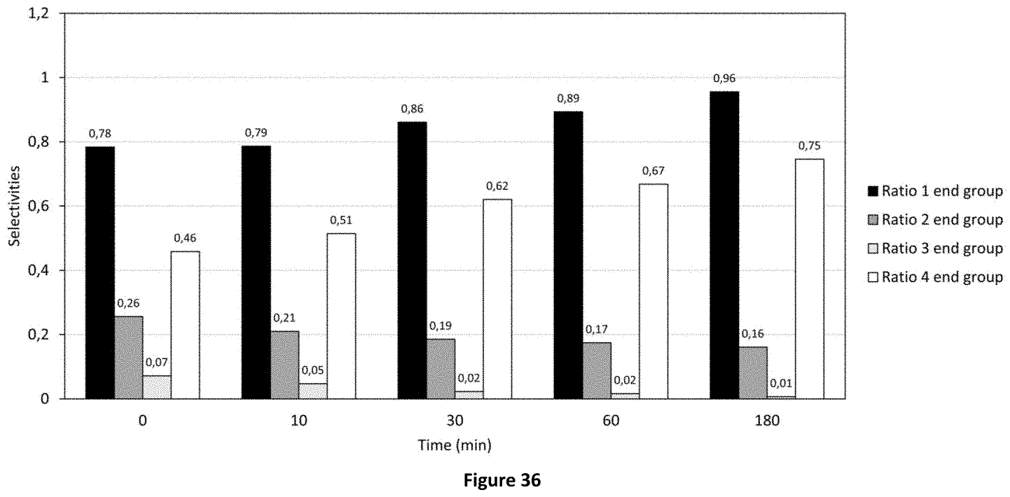

Detailed results on the molecular composition of the different groups are provided in Table 7 and FIGS. 25-36.

### Invention Examples 31-42

The lignin oils of the examples 31-42 were obtained using the general procedures of general methods and materials and the specific reaction conditions as specified in Table 8. The resulting lignin oils were analyzed according to the procedure in the general methods and materials. The examples 31-42, using a Ni/Al₂O₃ catalyst on 2 different types of biomass with varying reaction times, varying reaction temperature and varying hydrogen pressure are put forth to establish similarities in ratio's (ratio β-5, ratio β-β, ratio β-1 and ratio 1 end-groups) irrespective of the biomass or reaction conditions (temperature, solvent) and irrespective of the redox catalyst (in comparison with the compositions of invention examples 1-30).

Ratio β-1 was higher than 0.25 for all the compositions of invention examples 31-42 and was higher than 0.65 except for the compositions of invention examples 40 and 41. Compared with the compositions of comparative examples 1-6, this ratio was clearly higher. This higher ratio was the result of changes in the molecular composition. As shown by compositions of invention examples 31-42, the relative abundances of the unique β-1 E and, β-1 γ-OH structures could be tuned, depending on the desired properties.

Ratio β-5 was higher than 0.2 for all the compositions of invention examples 31-42. Compared with the compositions of comparative examples 1-6, this ratio was higher. This higher ratio was the result of changes in the molecular composition. As shown by the compositions of invention examples 31-42, the relative abundances of the unique β-5 E and β-5 γ-OH structures could be tuned, depending on the desired properties.

Ratio β-β was higher than 0.4 for the compositions of invention examples 31-34 and 40-42. The compositions of invention examples 35-39 are included to show the necessity of the correct combination of catalyst metal—biomass feedstock to obtain the desired ratio β-β.

The ratio 1 end-groups was higher than 0.5 for all the compositions of invention examples 31-42 and could be increased by increasing the reaction time. Furthermore, it is shown that the ratio 2 end-groups is higher than 0.35 and could be easily increased by altering the reaction conditions. In contrast, ratio 4 end-groups were lower than 0.2 for all the compositions of invention examples 31-42.

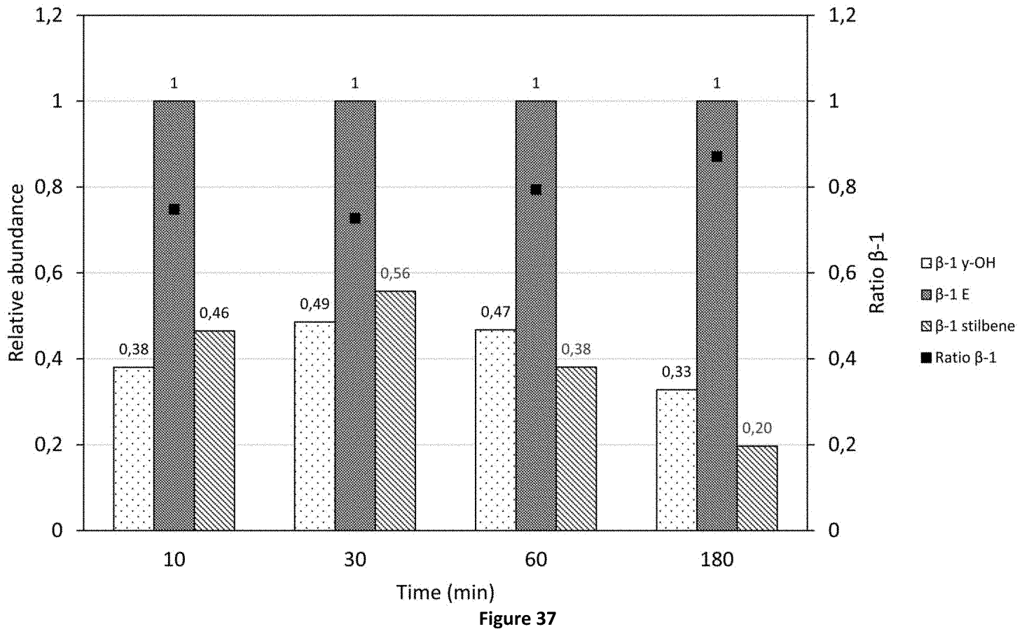

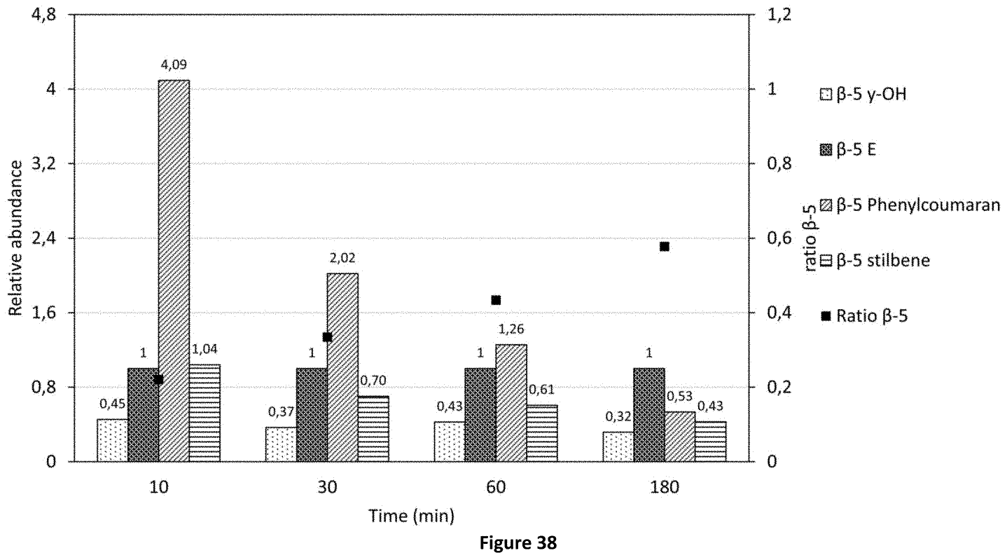

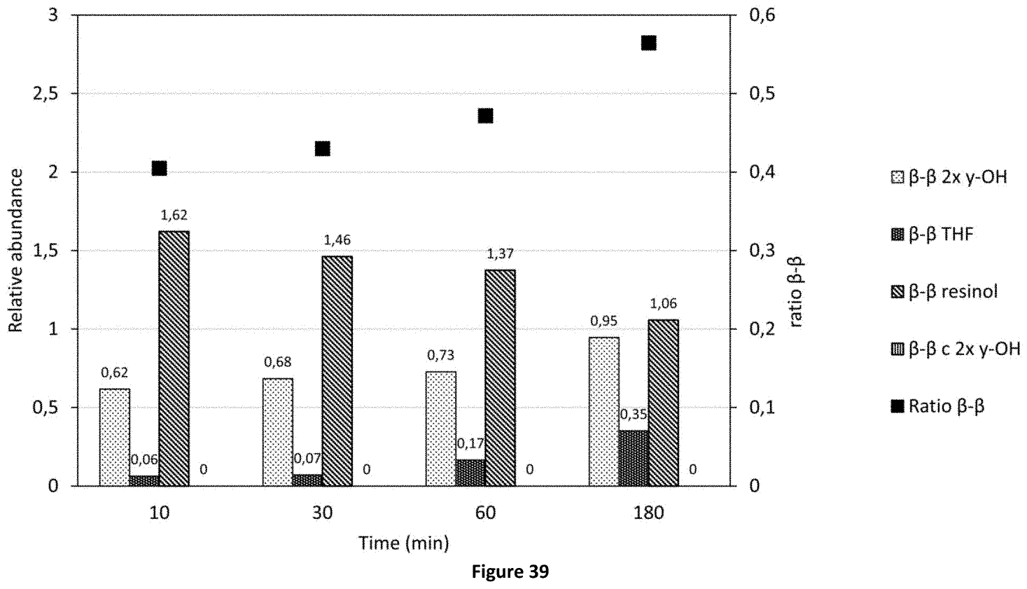

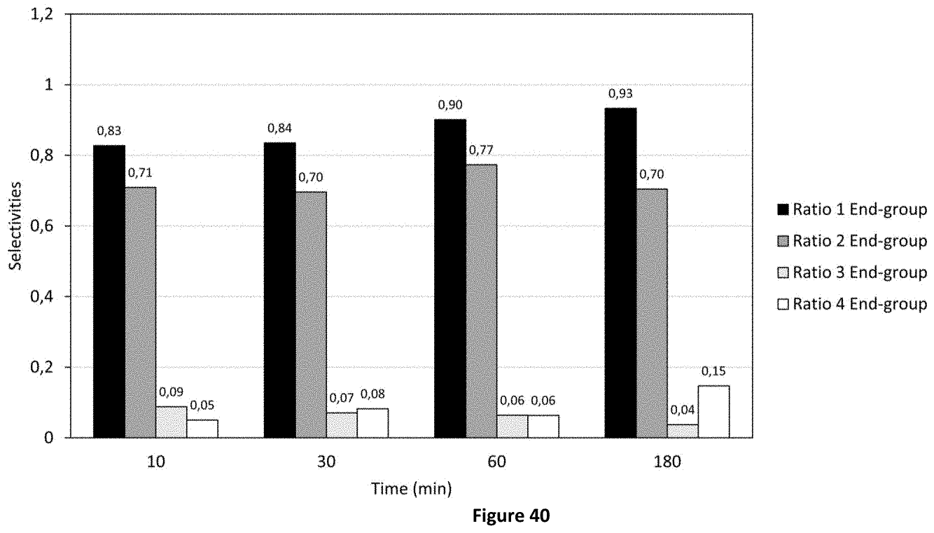

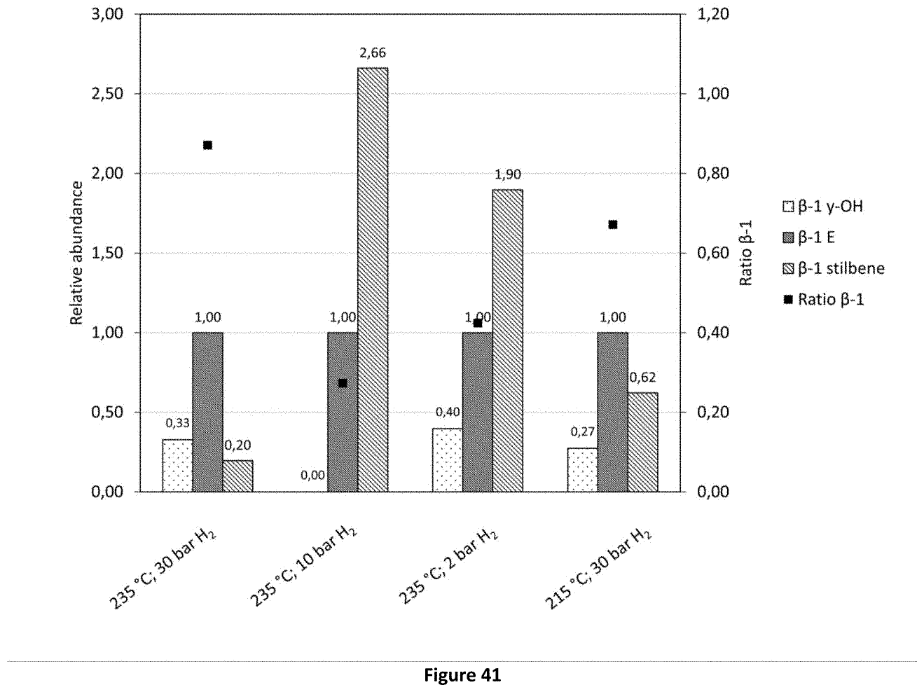

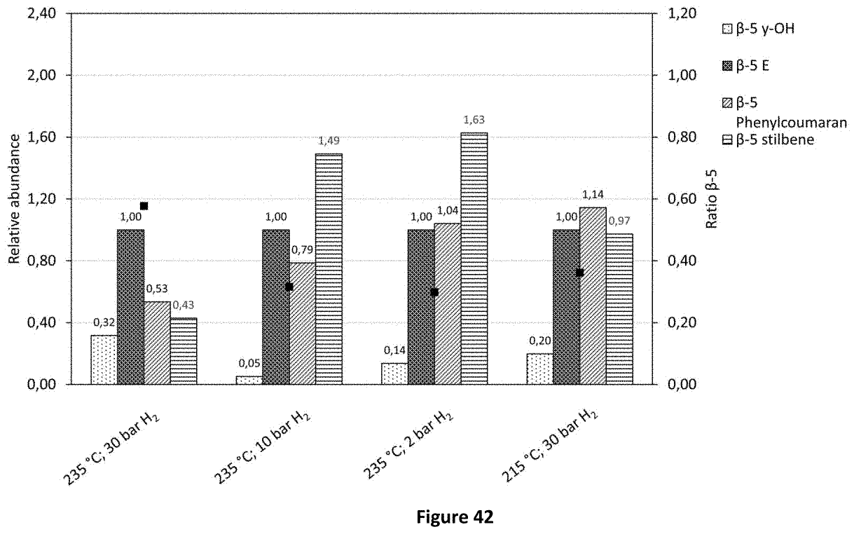

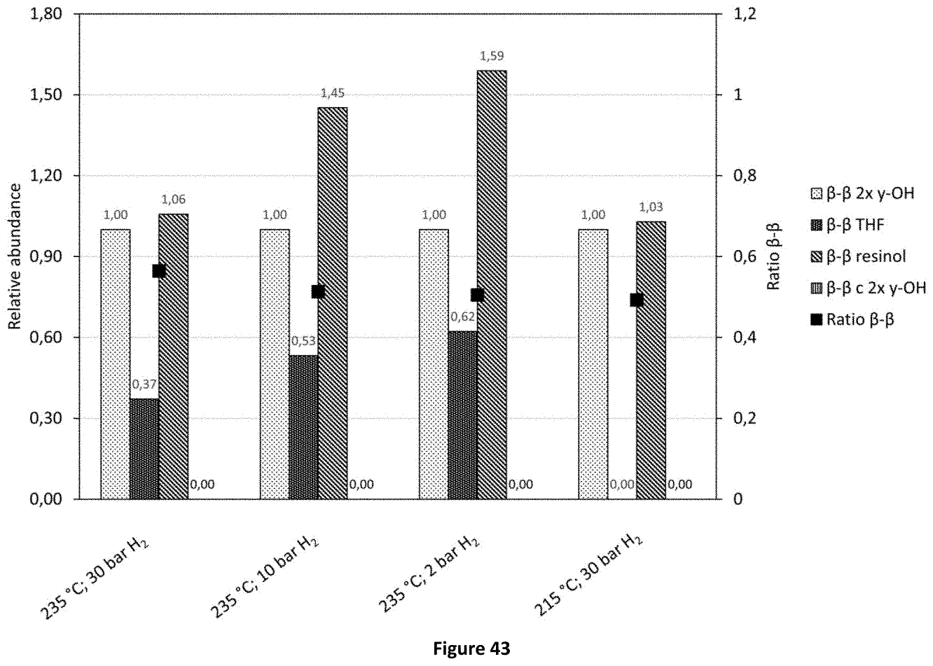

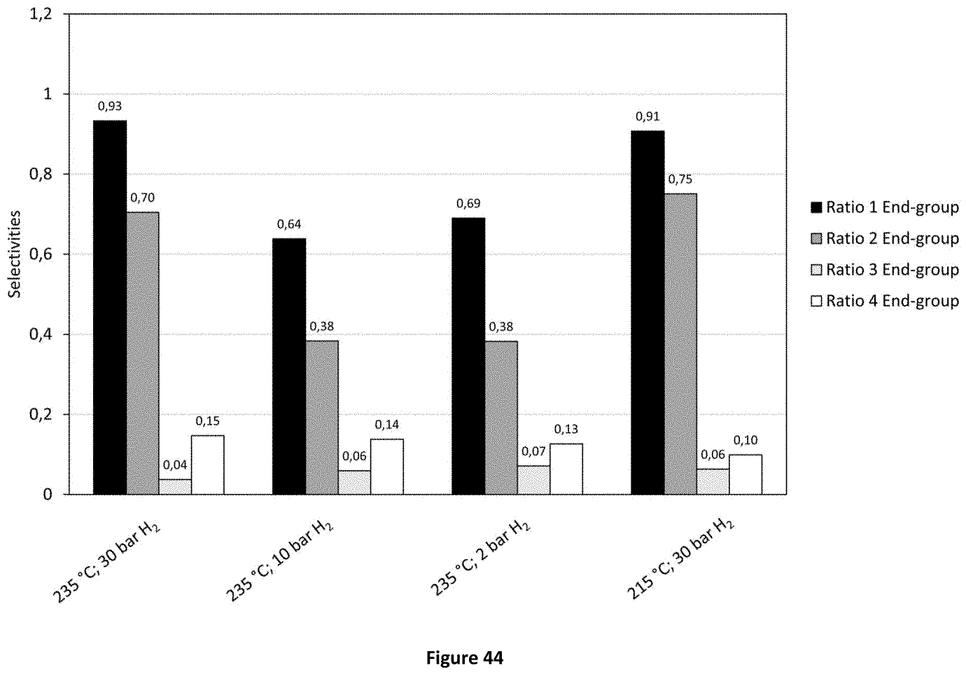

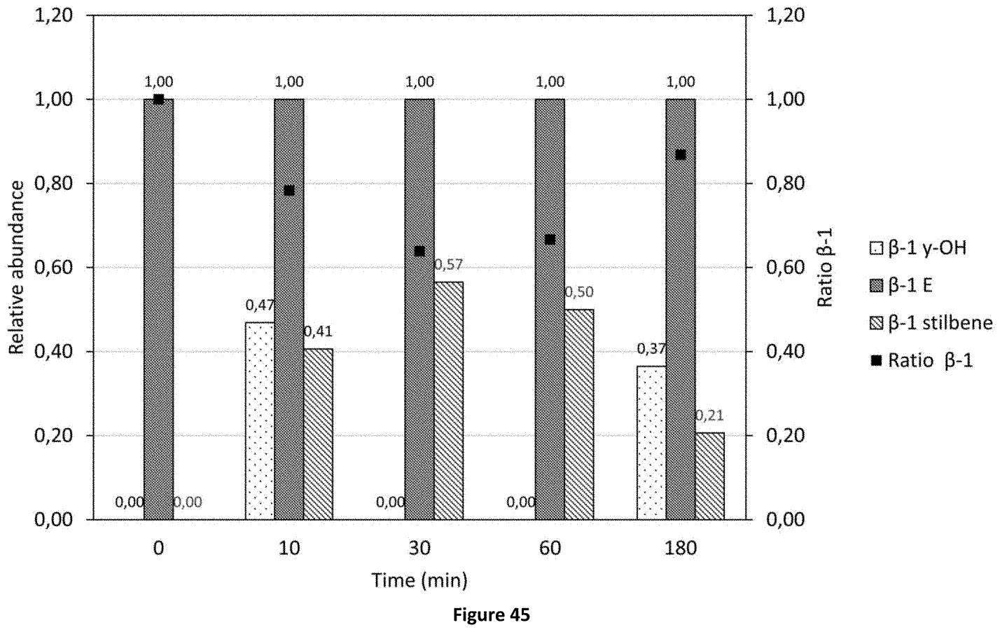

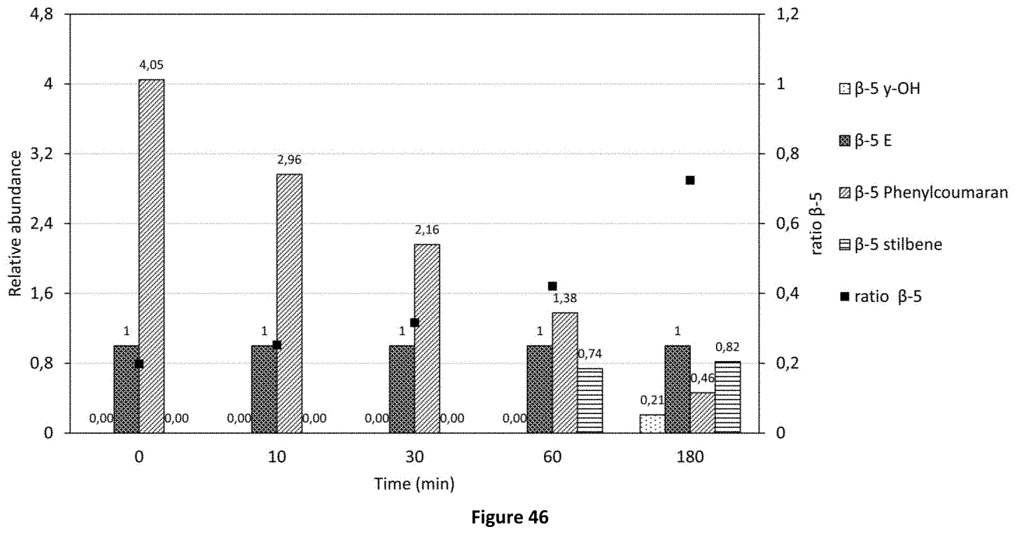

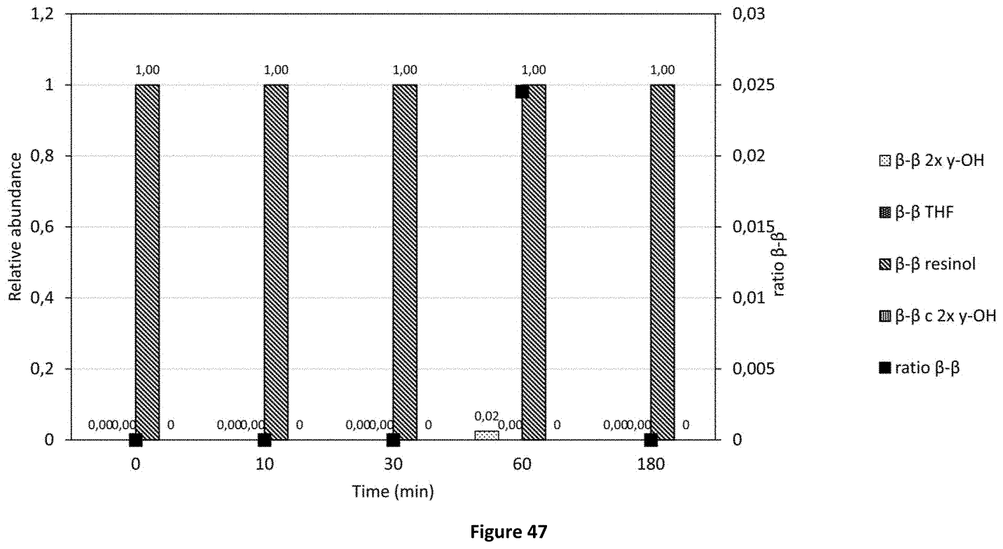

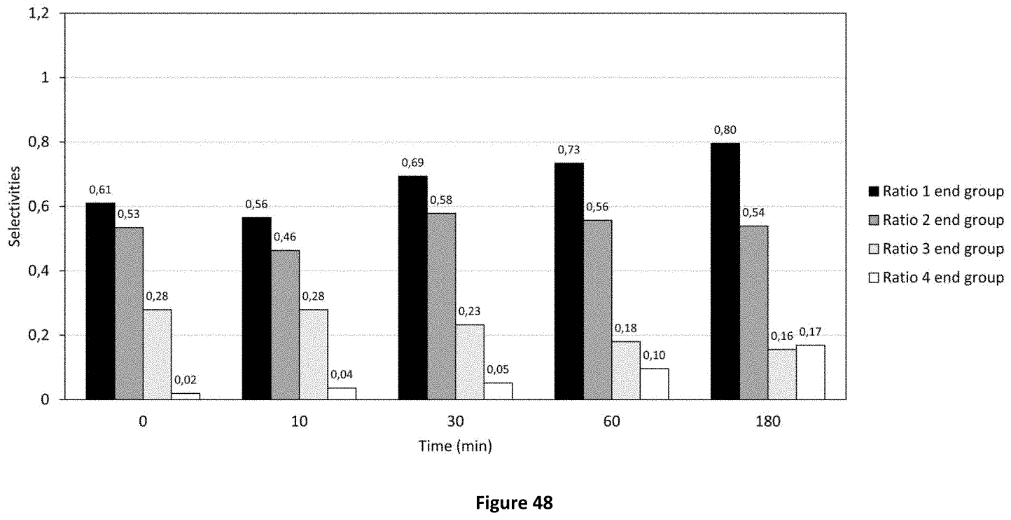

Detailed results on the molecular composition of the different groups are provided in Table 9 and FIGS. 37-48

### Invention Examples 43-50—Determination of the Hydroxyl Content of the Compositions of Invention Examples 5, 16, 21, 26, 34 and 39 and Comparative Examples 3 and 6

Invention examples 43-50 are included to show the effect of the different molecular compositions (of the compositions of different invention examples and comparative examples) on the amount of phenolic OH-units and aliphatic OH-units per gram of lignin oil, which is an important parameter to determine its reactivity and for its valorization.

Reactions with Pd/C as a catalyst resulted in compositions with the highest total OH content, which was mainly a consequence of its high ratio 1 end-groups and its high ratio 2 end-groups combined with a high ratio β-1, ratio β-5 and ratio β-β.

Reactions with Ru/C as a catalyst resulted in compositions with the lowest total OH content of the reactions with a catalyst. However, its phenolic OH content was similar to Pd/C. The lower OH content was mainly a consequence of the lower aliphatic OH content due to the combination of high ratio 1 end-groups, low ratio 2 end-groups, high ratio 4 end-groups and a high ratio β-1, ratio β-5 and ratio β-β.

Reactions with Ni/Al₂O₃ as a catalyst resulted in compositions with an intermediate OH content, with similar numbers of phenolic OH and aliphatic OH. Overall, this was the result of a slightly lower ratio 1 end-groups, intermediate ratio 2 end-groups, intermediate ratio 4 end-groups, an intermediate to high ratio β-1 and ratio β-5 and a low ratio of ratio β-β.

Clearly, when no catalyst was added, compositions with lower OH contents were obtained.

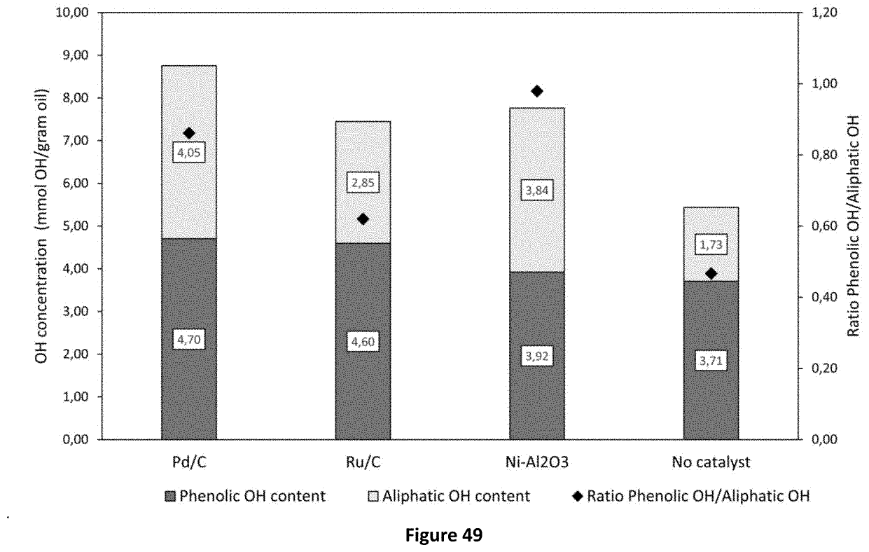

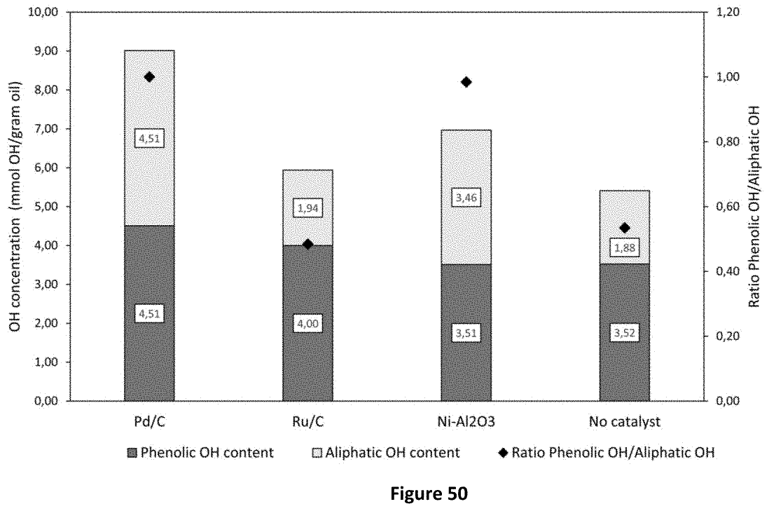

Detailed results on the hydroxyl content of the compositions of invention examples 5, 16, 24, 26, 34 and 39 and comparative examples 3 and 6 are provided in FIGS. 49-50.

### Invention Example 51

Determination of the Molecular Weight Distribution

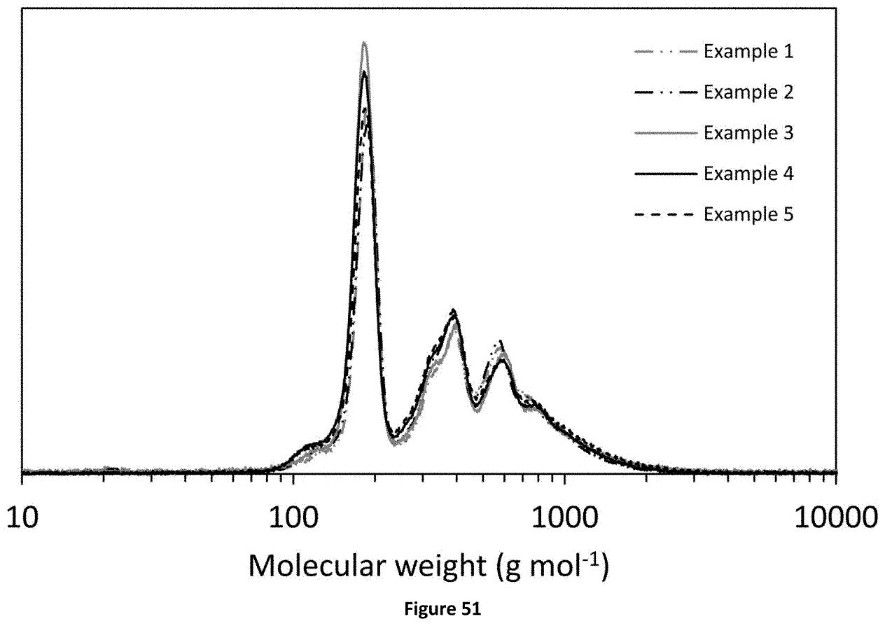

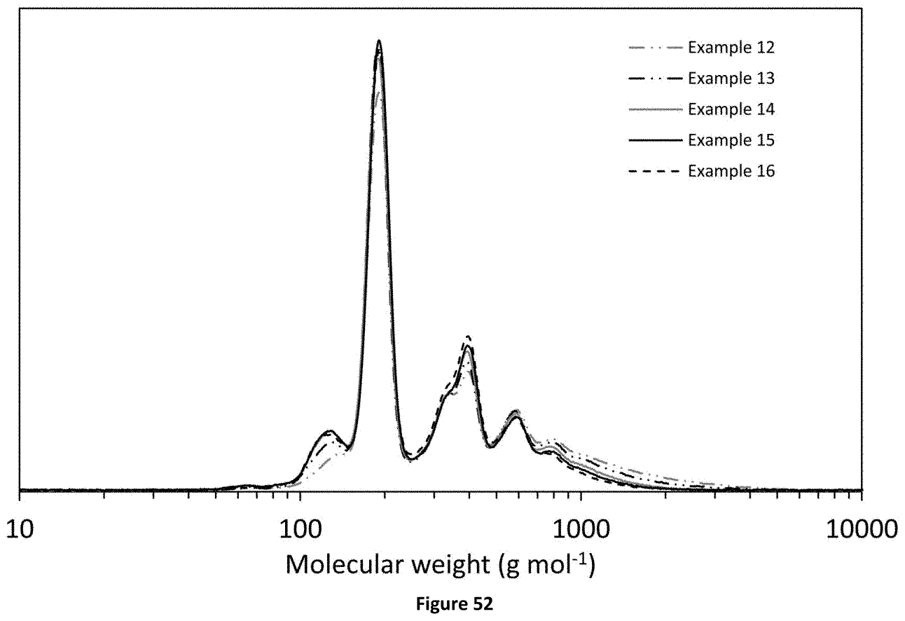

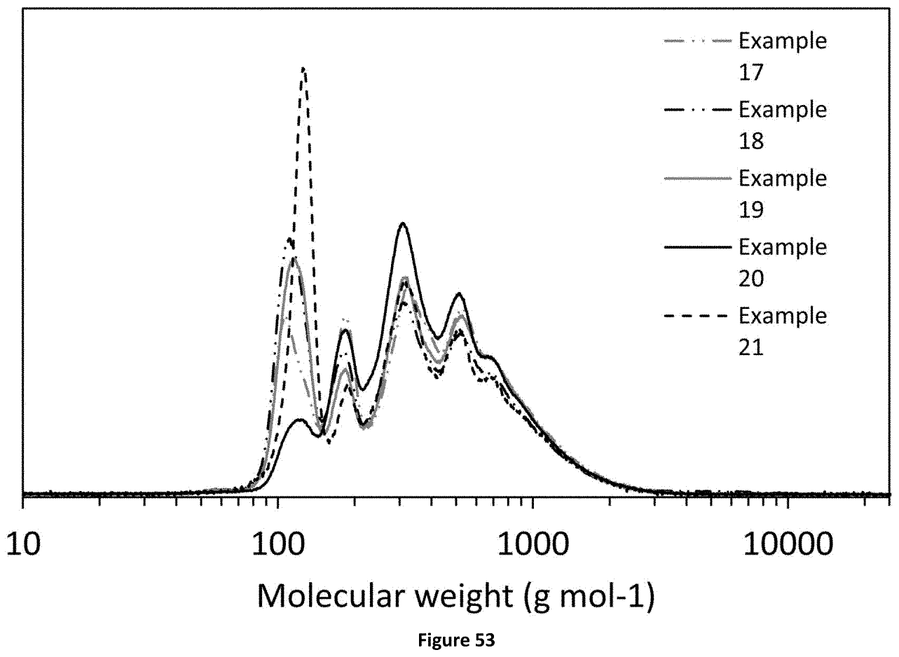

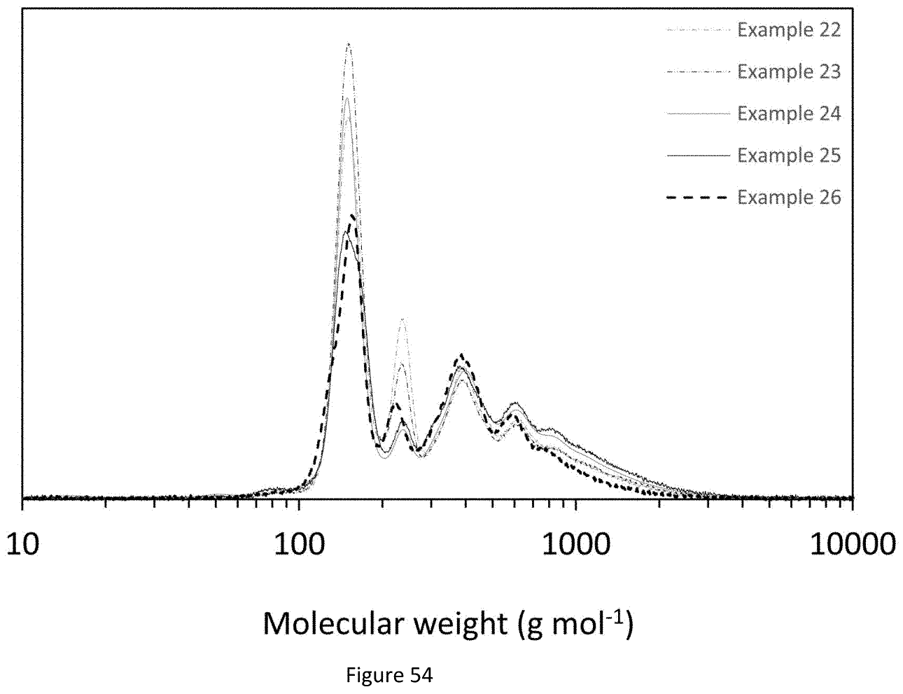

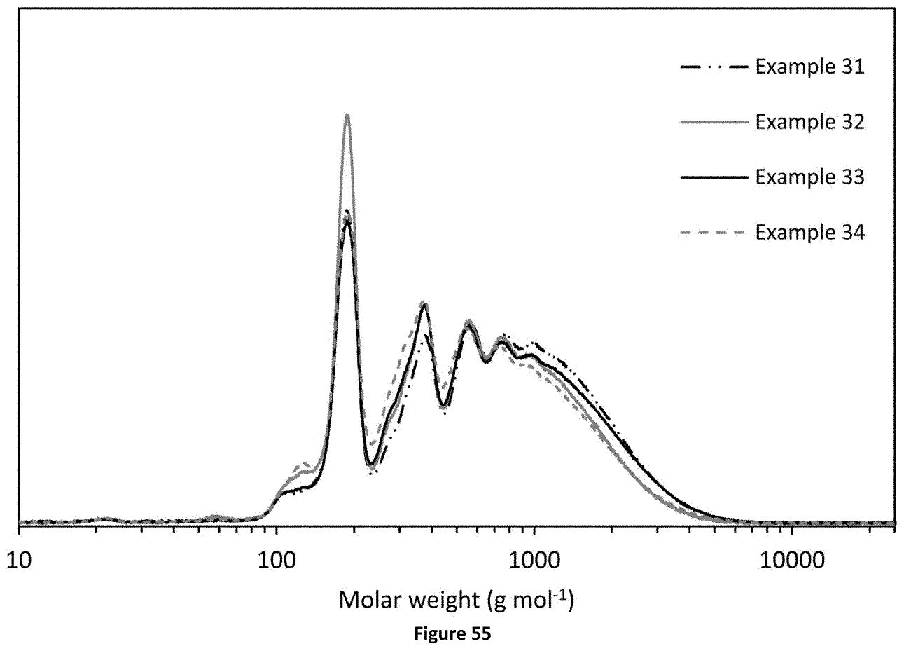

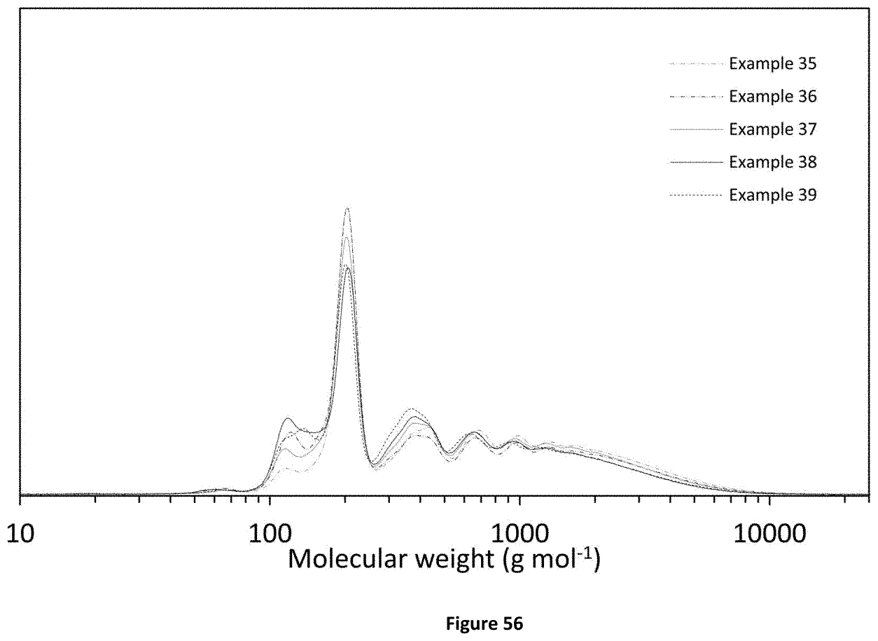

Invention example 51 is included to show the absence of molecular weight fragments higher than 10000 g/mol for lignin with the specific ratio's, as specified in the claims and the detailed description. To this end Gel Permeation Chromatograms (GPC) were obtained according to the method described in the preparation section. GPC's of the compositions of invention examples 1-5; 12-16; 17-21; 22-26; 31-34; and 35-39 are shown in FIG. 51-56. As can be seen from all Gel Permeation Chromatograms, no fragments with a molecular weight higher than 10000 g/mol were present in the compositions according to the present invention.

### Example 52

Invention example 52 is included to show the added benefit of the bioaromatic compositions. To this end, ³¹P of a phospholane chloride (2-chloro-4,4,5,5-tetramethyl-1,3,2-dioxaphospholane) was introduced into the lignin backbone by reacting the phospholane chloride in the presence of pyridine with the compositions of invention examples 5, 16, 21, 26, 34, and 39 and reference examples 3 and 6, using cholesterol as an internal standard. The amount of ³¹P incorporation was quantified by ³¹P-NMR and expressed as mmol ³¹P per gram of lignin. The results are shown in Table 10. Clearly, more phosphorous could be incorporated in the compositions according to the present invention, indicating the potential added benefit of these compositions in applications such as flame retardants (see D. Ghislain, et al., Polymer Chemistry, 2015, 6, 6257-6291).

### Example 53

Invention example 53 is included to show the added benefit of the bioaromatic composition. To this end, urethane groups were introduced on the aliphatic hydroxyl chains of the lignin backbone, by reacting the bioaromatic compositions of invention examples 5, 16, 26, and 39 with 2-naphtylisocyanate in the presence of trimethylamine. The amount of urethane linkages formed was quantified by ¹H-NMR and expressed as the number of urethane linkages per aromatic moiety. The results are shown in Table 11. Clearly, more urethane linkages per aromatic moiety could be incorporated in the bioaromatic compositions of the present invention, indicating the potential added benefit of these mixtures for polymer applications, such as polyurethanes.

Particular and preferred aspects of the invention are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this invention.

### Tables for this Application

TABLE 1
¹H and ¹³C NMR assignments of the diagnostic signals
for the different structures. These diagnostic
signals were used for quantification.

| Compound | Carbon atom | δ_{C} (ppm) | δ_{H} (ppm) | Factor |
|---|---|---|---|---|
| β-β resinol | α | 85 | 4.63 | 1 |
| β-β 2x γ-OH | β | 42 | 1.85 | 1 |
| β-β epiresinol | α | 87 | 4.34 | 0.5 |
| β-β 2x γ-OH condensed | | 46.5 | 1.87 | 0.5 |
| β-β THF | β | 46 | 2.09 | 1 |
| β-1 γ-OH | β | 49.2 | 2.8 | 1 |
| β-1 stilbene | α + β | 125.6 | 6.97 | 2 |
| β-1 E | α + β | 36.8 | 2.7 | 4 |
| β-5 Phenylcoumarane | α | 86.8 | 5.5 | 1 |
| β-5 γ-OH | β | 42.4 | 3.35 | 1 |
| β-5 stilbene | β | 120.1 | 7.22 | 1 |
| β-5 E | β | 32 | 2.7 | 2 |
| 4-propanol | β | 34.3 | 1.67 | 2 |
| 4-(3-methoxypropyl) | β | 30.8 | 1.74 | 2 |
| 4-propenol | γ | 61 | 4 | 2 |
| 4-propyl | β | 24.8 | 1.57 | 2 |
| 4-ethyl | α | 28.7 | 2.5 | 2 |
| 4-methyl | α | 20.4 | 2.22 | 3 |
| 4-(3-methoxypropenyl) | | 72.2 | 3.98 | 2 |
| 4-propenyl | | 17.9 | 1.77 | 3 |
| β-O-4 | α | 70-74 | 5-4.7 | 1 |
| β-O-4 - α-Me | α | 82 | 4.5 | 1 |
| β-O-4 red | β | 80.6 | 4.32 | 1 |

TABLE 2
Overview of reaction conditions for compositions of comparative examples 1-6 and their respective ratio

| | | | | | | | | Gas pressure | |
|---|---|---|---|---|---|---|---|---|---|
| | | | | | Catalyst | | | at room | Biomass |
| Comparative | | Duration* | Temperature | | amount | | | temperature | amount |
| example | Substrate | [min] | [° C.] | Catalyst | [g] | Solvent | Gas | [bar] | [g] |
| 1 | pine | 0 | 235 | — | — | MeOH | H₂ | 30 | 2.0 |
| 2 | pine | 30 | 235 | — | — | MeOH | H₂ | 30 | 2.0 |
| 3 | pine | 180 | 235 | — | — | MeOH | H₂ | 30 | 2.0 |
| 4 | Birch | 0 | 235 | — | — | MeOH | H₂ | 30 | 2.0 |
| 5 | Birch | 30 | 235 | — | — | MeOH | H₂ | 30 | 2.0 |
| 6 | Birch | 180 | 235 | — | — | MeOH | H₂ | 30 | 2.0 |

| | | Oil | | | | | | | | |
|---|---|---|---|---|---|---|---|---|---|---|
| | Biomass/ | production/ | | | | | Ratio | Ratio | Ratio | Ratio |
| Comparative | catalyst | wood | % wood | Ratio | Ratio | Ratio | 1-end | 2-end | 3-end | 4 end- |
| example | wt ratio | [wt ratio] | retention | β-1 | β-5 | β-β | groups | groups | groups | groups |
| 1 | — | 0.04 | 0.91 | 0.11 | 0.00 | 0.29 | 0.10 | 0.07 | 0.38 | 0.00 |
| 2 | — | 0.07 | 0.85 | 0.10 | 0.00 | 0.43 | 0.11 | 0.08 | 0.21 | 0.00 |
| 3 | — | 0.08 | 0.81 | 0.11 | 0.00 | 0.52 | 0.29 | 0.21 | 0.19 | 0.00 |
| 4 | — | 0.04 | 0.86 | 0.00 | 0.00 | 0.02 | 0.03 | 0.03 | 0.50 | 0.00 |
| 5 | — | 0.08 | 0.80 | 0.00 | 0.00 | 0.07 | 0.02 | 0.02 | 0.37 | 0.00 |
| 6 | — | 0.15 | 0.66 | 0.00 | 0.00 | 0.00 | 0.13 | 0.09 | 0.33 | 0.00 |

*Duration at reaction temperature

TABLE 3
Relative abundance of molecular structures for compositions
of comparative examples 1-6 ad their respective ratio's.

| | | | | | | | β-5 | | Ratio | β-β |
|---|---|---|---|---|---|---|---|---|---|---|
| Comparative | β-1 | β-1 | β-1 | Ratio | β-5 | β-5 | Phenyl- | β-5 | β-5 | 2x |
| example | y-OH | E | stilbene | β-1 | y-OH | E | coumaran | stilbene | γ-OH | y-OH |
| 1 | 0.00 | 1.00 | 8.17 | 0.11 | 0.00 | 0.00 | 1.00 | 0.00 | 0.00 | 0.26 |
| 2 | 0.00 | 1.00 | 8.94 | 0.10 | 0.00 | 0.00 | 1.00 | 0.75 | 0.00 | 0.56 |
| 3 | 0.00 | 1.00 | 8.32 | 0.11 | 0.00 | 0.00 | 1.00 | 3.42 | 0.00 | 0.77 |
| 4 | 0.00 | 0.00 | 100 | 0.00 | 0.00 | 0.00 | 1.00 | 0.00 | 0.00 | 0.02 |
| 5 | 0.00 | 0.00 | 1.00 | 0.00 | 0.00 | 0.00 | 1.00 | 0.00 | 0.00 | 0.07 |
| 6 | 0.00 | 0.00 | 1.00 | 0.00 | 0.00 | 0.00 | 0.00 | 0.00 | 0.00 | 0.00 |

| | | β-β | | | Ratio | Ratio | Ratio | Ratio |
|---|---|---|---|---|---|---|---|---|
| Comparative | β-β | c 2x | β-β | Ratio | 1 end- | 2 end- | 3 end- | 4 end- |
| example | THF | y-OH | resinol | β-β | groups | groups | groups | groups |
| 1 | 0.15 | 0.00 | 1.00 | 0.29 | 0.10 | 0.07 | 0.38 | 0.00 |
| 2 | 0.20 | 0.00 | 1.00 | 0.43 | 0.11 | 0.08 | 0.21 | 0.00 |
| 3 | 0.31 | 0.00 | 1.00 | 0.52 | 0.29 | 0.21 | 0.19 | 0.00 |
| 4 | 0.00 | 0.00 | 1.00 | 0.02 | 0.03 | 0.03 | 0.50 | 0.00 |
| 5 | 0.00 | 0.00 | 1.00 | 0.07 | 0.02 | 0.02 | 0.37 | 0.00 |
| 6 | 0.00 | 0.00 | 1.00 | 0.00 | 0.13 | 0.09 | 0.33 | 0.00 |

TABLE 4
Overview of reaction conditions for compositions of invention examples 1-16 and their respective ratio's.

| | | | | | | | | Gas pressure | |
|---|---|---|---|---|---|---|---|---|---|
| | | | | | Catalyst | | | at room | Biomass |
| Invention | | Duration* | Temperature | | amount | | | temperature | amount |
| example | Substrate | [min] | [° C.] | Catalyst | [g] | Solvent | Gas | [bar] | [g] |
| 1 | pine | 0 | 235 | Pd/C | 0.20 | MeOH | H₂ | 30 | 2.0 |
| 2 | pine | 10 | 235 | Pd/C | 0.20 | MeOH | H₂ | 30 | 2.0 |
| 3 | pine | 30 | 235 | Pd/C | 0.20 | MeOH | H₂ | 30 | 2.0 |
| 4 | pine | 60 | 235 | Pd/C | 0.20 | MeOH | H₂ | 30 | 2.0 |
| 5 | pine | 180 | 235 | Pd/C | 0.20 | MeOH | H₂ | 30 | 2.0 |
| 6 | pine | 180 | 235 | Pd/C | 0.20 | MeOH | H₂ | 10 | 2.0 |
| 7 | pine | 180 | 215 | Pd/C | 0.20 | MeOH | H₂ | 30 | 2.0 |
| 8 | pine | 180 | 235 | Pd/C | 0.20 | MeOH | H₂ | 2 | 2.0 |
| 9 | pine | 180 | 215 | Pd/C | 0.20 | MeOH | H₂ | 10 | 2.0 |
| 10 | pine | 180 | 215 | Pd/C | 0.20 | MeOH | H₂ | 2 | 2.0 |
| 11 | Pine | 180 | 195 | Pd/C | 0.20 | MeOH | H₂ | 30 | 2.0 |
| 12 | Birch | 0 | 235 | Pd/C | 0.20 | MeOH | H₂ | 30 | 2.0 |
| 13 | Birch | 10 | 235 | Pd/C | 0.20 | MeOH | H₂ | 30 | 2.0 |
| 14 | Birch | 30 | 235 | Pd/C | 0.20 | MeOH | H₂ | 30 | 2.0 |
| 15 | Birch | 60 | 235 | Pd/C | 0.20 | MeOH | H₂ | 30 | 2.0 |
| 16 | Birch | 180 | 235 | Pd/C | 0.20 | MeOH | H₂ | 30 | 2.0 |

| | | Oil | | | | | | | | |
|---|---|---|---|---|---|---|---|---|---|---|
| | Biomass/ | production/ | wt % | | | | Ratio | Ratio | Ratio | Ratio |
| Invention | catalyst | wood | wood | Ratio | Ratio | Ratio | 1-end | 2-end | 3-end | 4 end- |
| example | wt ratio | [wt ratio] | retention | β-1 | β-5 | β-β | groups | groups | groups | groups |
| 1 | 10 | 0.04 | 0.89 | 1.00 | 0.92 | 1.00 | 0.96 | 0.87 | 0.03 | 0.03 |
| 2 | 10 | 0.07 | 0.83 | 1.00 | 1.00 | 1.00 | 0.96 | 0.86 | 0.02 | 0.04 |
| 3 | 10 | 0.09 | 0.88 | 1.00 | 1.00 | 1.00 | 0.97 | 0.87 | 0.02 | 0.04 |
| 4 | 10 | 0.09 | 0.74 | 1.00 | 1.00 | 1.00 | 0.97 | 0.87 | 0.02 | 0.04 |
| 5 | 10 | 0.11 | 0.72 | 1.00 | 1.00 | 1.00 | 0.97 | 0.83 | 0.01 | 0.06 |
| 6 | 10 | 0.10 | 0.76 | 1.00 | 1.00 | 1.00 | 0.96 | 0.63 | 0.03 | 0.08 |
| 7 | 10 | 0.10 | 0.84 | 1.00 | 1.00 | 1.00 | 0.97 | 0.87 | 0.02 | 0.04 |
| 8 | 10 | 0.10 | 0.74 | 1.00 | 1.00 | 1.00 | 0.95 | 0.49 | 0.03 | 0.10 |
| 9 | 10 | 0.09 | 0.81 | 1.00 | 1.00 | 1.00 | 0.96 | 0.81 | 0.02 | 0.06 |
| 10 | 10 | 0.08 | 0.84 | 1.00 | 1.00 | 1.00 | 0.95 | 0.54 | 0.03 | 0.09 |
| 11 | 10 | 0.07 | 0.88 | 1.00 | 1.00 | 1.00 | 0.97 | 0.87 | 0.02 | 0.04 |
| 12 | 10 | 0.04 | 0.83 | 1.00 | 0.85 | 0.53 | 0.91 | 0.82 | 0.09 | 0.03 |
| 13 | 10 | 0.08 | 0.82 | 1.00 | 0.96 | 0.65 | 0.91 | 0.83 | 0.08 | 0.03 |
| 14 | 10 | 0.11 | 0.73 | 1.00 | 1.00 | 0.96 | 0.95 | 0.87 | 0.04 | 0.03 |
| 15 | 10 | 0.13 | 0.69 | 1.00 | 1.00 | 1.00 | 0.97 | 0.89 | 0.02 | 0.04 |
| 16 | 10 | 0.16 | 0.62 | 1.00 | 1.00 | 1.00 | 0.98 | 0.89 | 0.01 | 0.03 |

*Duration at reaction temperature

TABLE 5
Relative abundance of molecular structures for the compositions
of invention examples 1-16 and their respective ratio's.

| | | | | | | | | | Ratio | |
|---|---|---|---|---|---|---|---|---|---|---|
| Invention | β-1 | β-1 | β-1 | Ratio | β-5 | β-5 | β-5 Phenyl- | β-5 | β-5 | β-β 2x |
| example | y-OH | E | stilbene | β-1 | y-OH | E | coumaran | stilbene | γ-OH | y-OH |
| 1 | 3.28 | 1.00 | 0.00 | 1.00 | 3.26 | 1.00 | 0.36 | 0.00 | 1.00 | 1.00 |
| 2 | 3.44 | 1.00 | 0.00 | 1.00 | 2.5 | 1.00 | 0.00 | 0.00 | 1.00 | 1.00 |
| 3 | 2.81 | 1.00 | 0.00 | 1.00 | 1.65 | 1.00 | 0.00 | 0.00 | 1.00 | 1.00 |
| 4 | 2.26 | 1.00 | 0.00 | 1.00 | 1.1 | 1.00 | 0.00 | 0.00 | 1.00 | 1.00 |
| 5 | 1.48 | 1.00 | 0.00 | 1.00 | 0.94 | 1.00 | 0.00 | 0.00 | 1.00 | 1.00 |
| 6 | 1.01 | 1.00 | 0.00 | 1.00 | 0.95 | 1.00 | 0.00 | 0.00 | 1.00 | 1.00 |
| 7 | 0.60 | 1.00 | 0.00 | 1.00 | 0.72 | 1.00 | 0.00 | 0.00 | 1.00 | 1.00 |
| 8 | 2.56 | 1.00 | 0.00 | 1.00 | 1.62 | 1.00 | 0.00 | 0.00 | 1.00 | 1.00 |
| 9 | 2.30 | 1.00 | 0.00 | 1.00 | 1.76 | 1.00 | 0.00 | 0.00 | 1.00 | 1.00 |
| 10 | 1.04 | 1.00 | 0.00 | 1.00 | 1.20 | 1.00 | 0.00 | 0.00 | 1.00 | 1.00 |
| 11 | 4.34 | 1.00 | 0.00 | 1.00 | 3.49 | 1.00 | 0.00 | 0.00 | 1.00 | 1.00 |
| 12 | 1.27 | 1.00 | 0.00 | 1.00 | 1.31 | 1.00 | 0.40 | 0.00 | 0.85 | 1.00 |
| 13 | 1.65 | 1.00 | 0.00 | 1.00 | 0.86 | 1.00 | 0.07 | 0.00 | 0.96 | 1.00 |
| 14 | 1.46 | 1.00 | 0.00 | 1.00 | 1.01 | 1.00 | 0.00 | 0.00 | 1.00 | 1.00 |
| 15 | 1.64 | 1.00 | 0.00 | 1.00 | 0.91 | 1.00 | 0.00 | 0.00 | 1.00 | 1.00 |
| 16 | 1.51 | 1.00 | 0.00 | 1.00 | 0.74 | 1.00 | 0.00 | 0.00 | 1.00 | 1.00 |

| | | | | | Ratio | Ratio | Ratio | Ratio |
|---|---|---|---|---|---|---|---|---|
| Invention | β-β | β-β c 2x | β-β | Ratio | 1 end- | 2 end- | 3 end- | 4 end- |
| example | THF | y-OH | resinol | β-β | groups | groups | groups | groups |
| 1 | 0.07 | 0.00 | 0.00 | 1.00 | 0.96 | 0.87 | 0.03 | 0.03 |
| 2 | 0.08 | 0.00 | 0.00 | 1.00 | 0.96 | 0.86 | 0.02 | 0.04 |
| 3 | 0.12 | 0.00 | 0.00 | 1.00 | 0.97 | 0.87 | 0.02 | 0.04 |
| 4 | 0.16 | 0.00 | 0.00 | 1.00 | 0.97 | 0.87 | 0.02 | 0.04 |
| 5 | 0.28 | 0.00 | 0.00 | 1.00 | 0.97 | 0.83 | 0.01 | 0.06 |
| 6 | 0.51 | 0.00 | 0.00 | 1.00 | 0.96 | 0.63 | 0.03 | 0.08 |
| 7 | 1.02 | 0.00 | 0.00 | 1.00 | 0.97 | 0.87 | 0.02 | 0.04 |
| 8 | 0.12 | 0.00 | 0.00 | 1.00 | 0.95 | 0.49 | 0.03 | 0.10 |
| 9 | 0.19 | 0.00 | 0.00 | 1.00 | 0.96 | 0.81 | 0.02 | 0.06 |
| 10 | 0.35 | 0.00 | 0.00 | 1.00 | 0.95 | 0.54 | 0.03 | 0.09 |
| 11 | 0.13 | 0.00 | 0.00 | 1.00 | 0.97 | 0.87 | 0.02 | 0.04 |
| 12 | 0.00 | 0.00 | 0.88 | 1.00 | 0.91 | 0.82 | 0.09 | 0.03 |
| 13 | 0.03 | 0.00 | 0.55 | 0.65 | 0.91 | 0.83 | 0.08 | 0.03 |
| 14 | 0.04 | 0.00 | 0.05 | 0.96 | 0.95 | 0.87 | 0.04 | 0.03 |
| 15 | 0.04 | 0.00 | 0.00 | 1.00 | 0.97 | 0.89 | 0.02 | 0.04 |
| 16 | 0.13 | 0.00 | 0.00 | 1.00 | 0.98 | 0.89 | 0.01 | 0.03 |

TABLE 6
Overview of reaction conditions for the compositions of
invention examples 17-30 and their respective ratio's.

| | | | | | | | | Gas pressure | |
|---|---|---|---|---|---|---|---|---|---|
| | | | | | catalyst | | | at room | biomass |
| Invention | | Duration* | Temperature | | amount | | | temperature | amount |
| example | Substrate | (min) | (° C.) | Catalyst | (g) | solvent | Gas | (bar) | (g) |
| 17 | pine | 0 | 235 | Ru/C | 0.20 | MeOH | H₂ | 30 | 2.0 |
| 18 | pine | 10 | 235 | Ru/C | 0.20 | MeOH | H₂ | 30 | 2.0 |
| 19 | pine | 30 | 235 | Ru/C | 0.20 | MeOH | H₂ | 30 | 2.0 |
| 20 | pine | 60 | 235 | Ru/C | 0.20 | MeOH | H₂ | 30 | 2.0 |
| 21 | pine | 180 | 235 | Ru/C | 0.20 | MeOH | H₂ | 30 | 2.0 |
| 22 | Birch | 0 | 235 | Ru/C | 0.20 | MeOH | H₂ | 30 | 2.0 |
| 23 | Birch | 10 | 235 | Ru/C | 0.20 | MeOH | H₂ | 30 | 2.0 |
| 24 | Birch | 30 | 235 | Ru/C | 0.20 | MeOH | H₂ | 30 | 2.0 |
| 25 | Birch | 60 | 235 | Ru/C | 0.20 | MeOH | H₂ | 30 | 2.0 |
| 26 | Birch | 180 | 235 | Ru/C | 0.20 | MeOH | H₂ | 30 | 2.0 |
| 27 | Eucalyptus | 120 | 200 | Ru/C | 0.20 | BuOH/ | H₂ | 30 | 2.0 |
| | | | | | | Water | | | |
| | | | | | | (1/1) | | | |
| 28 | Eucalyptus | 180 | 250 | Ru/C | 0.20 | MeOH | H₂ | 30 | 2.0 |
| 29 | pine | 120 | 200 | Ru/C | 0.20 | BuOH/ | H₂ | 30 | 2.0 |
| | | | | | | Water | | | |
| | | | | | | (1/1) | | | |
| 30 | pine | 180 | 250 | Ru/C | 0.20 | MeOH | H₂ | 30 | 2.0 |

| | | oil | | | | | | | | |
|---|---|---|---|---|---|---|---|---|---|---|
| | Biomass/ | production/ | wt % | | | | Ratio | Ratio | Ratio | Ratio |
| Invention | catalyst | wood | wood | β-1 | β-5 | Ratio | 1 end- | 2 end- | 3 end- | 4 end- |
| example | wt ratio | [wt ratio] | retention | Ratio | Ratio | β-β | groups | groups | groups | groups |
| 17 | 10 | 0.03 | 0.85 | 1.00 | 0.70 | 0.77 | 0.82 | 0.44 | 0.05 | 0.29 |
| 18 | 10 | 0.05 | 0.83 | 0.97 | 0.86 | 0.76 | 0.71 | 0.23 | 0.01 | 0.43 |
| 19 | 10 | 0.08 | 0.83 | 0.97 | 0.93 | 0.90 | 0.78 | 0.16 | 0.01 | 0.57 |
| 20 | 10 | 0.09 | 0.77 | 1.00 | 0.99 | 1.00 | 0.83 | 0.23 | 0.01 | 0.56 |
| 21 | 10 | 0.11 | 0.72 | 1.00 | 1.00 | 1.00 | 0.90 | 0.19 | 0.00 | 0.65 |
| 22 | 10 | 0.04 | 0.84 | 1.00 | 0.55 | 0.05 | 0.78 | 0.26 | 0.07 | 0.46 |
| 23 | 10 | 0.07 | 0.91 | 1.00 | 0.74 | 0.26 | 0.79 | 0.21 | 0.05 | 0.51 |
| 24 | 10 | 0.10 | 0.77 | 1.00 | 0.86 | 0.47 | 0.86 | 0.19 | 0.02 | 0.62 |
| 25 | 10 | 0.12 | 0.72 | 1.00 | 0.94 | 0.70 | 0.89 | 0.17 | 0.02 | 0.67 |
| 26 | 10 | 0.15 | 0.66 | 1.00 | 1.00 | 0.96 | 0.96 | 0.16 | 0.01 | 0.75 |
| 27 | 10 | 0.21 | | 1.00 | 1.00 | 0.60 | 0.96 | 0.80 | 0.02 | 0.08 |
| 28 | 10 | 0.16 | | 1.00 | 1.00 | 1.00 | 0.96 | 0.08 | 0.00 | 0.80 |
| 29 | 10 | 0.16 | | 1.00 | 0.74 | 0.85 | 0.96 | 0.78 | 0.02 | 0.11 |
| 30 | 10 | 0.10 | | 1.00 | 1.00 | 1.00 | 0.94 | 0.22 | 0.01 | 0.67 |

*Duration at reaction temperature

TABLE 7
Relative abundance of molecular structures for
the compositions of invention examples 17-30

| | | | | | | | β-5 | | Ratio | |
|---|---|---|---|---|---|---|---|---|---|---|
| Invention | β-1 | β-1 | β-1 | Ratio | β-5 | β-5 | Phenyl- | β-5 | β-5 | β-β 2x |
| example | y-OH | E | stilbene | β-1 | y-OH | E | coumaran | stilbene | γ-OH | y-OH |
| 17 | 6.01 | 1.00 | 0.00 | 1.00 | 3.98 | 1.00 | 2.15 | 0.00 | 0.70 | 1.00 |
| 18 | 3.92 | 1.00 | 0.14 | 0.97 | 3.52 | 1.00 | 0.72 | 0.39 | 0.86 | 1.00 |
| 19 | 3.11 | 1.00 | 0.11 | 0.97 | 2.14 | 1.00 | 0.25 | 0.26 | 0.93 | 1.00 |
| 20 | 2.15 | 1.00 | 0.00 | 1.00 | 1.82 | 1.00 | 0.04 | 0.29 | 0.99 | 1.00 |
| 21 | 2.28 | 1.00 | 0.00 | 1.00 | 1.28 | 1.00 | 0.00 | 0.13 | 1.00 | 1.00 |
| 22 | 3.31 | 1.00 | 0.00 | 1.00 | 2.05 | 1.00 | 2.49 | 0.00 | 0.55 | 1.00 |
| 23 | 3.73 | 1.00 | 0.00 | 1.00 | 1.20 | 1.00 | 0.78 | 0.00 | 0.74 | 1.00 |
| 24 | 3.50 | 1.00 | 0.00 | 1.00 | 1.38 | 1.00 | 0.39 | 0.00 | 0.86 | 1.00 |
| 25 | 2.38 | 1.00 | 0.00 | 1.00 | 1.29 | 1.00 | 0.15 | 0.00 | 0.94 | 1.00 |
| 26 | 1.91 | 1.00 | 0.00 | 1.00 | 0.91 | 1.00 | 0.00 | 0.00 | 1.00 | 1.00 |
| 27 | 3.00 | 1.00 | 0.00 | 1.00 | 0.00 | 1.04 | 1.00 | 0.00 | 0.00 | 1.00 |
| 28 | 1.01 | 1.00 | 0.00 | 1.00 | 0.00 | 0.71 | 1.00 | 0.00 | 0.00 | 1.00 |
| 30 | 2.13 | 1.00 | 0.00 | 1.00 | 0.00 | 1.31 | 1.00 | 0.00 | 0.00 | 1.00 |
| 29 | 2.85 | 1.00 | 0.00 | 1.00 | 0.00 | 3.89 | 1.00 | 1.72 | 0.00 | 1.00 |

| | | | | | Ratio | Ratio | Ratio | Ratio |
|---|---|---|---|---|---|---|---|---|
| Invention | β-β | β-βc 2x | β-β | Ratio | 1 end- | 2 end- | 3 end- | 4 end- |
| example | THF | y-OH | resinol | β-β | groups | groups | groups | groups |
| 17 | 0.11 | 0.00 | 0.32 | 0.77 | 0.82 | 0.44 | 0.05 | 0.29 |
| 18 | 0.11 | 0.00 | 0.36 | 0.76 | 0.71 | 0.23 | 0.01 | 0.43 |
| 19 | 0.30 | 0.00 | 0.14 | 0.90 | 0.78 | 0.16 | 0.01 | 0.57 |
| 20 | 0.15 | 0.00 | 0.00 | 1.00 | 0.83 | 0.23 | 0.01 | 0.56 |
| 21 | 0.31 | 0.00 | 0.00 | 1.00 | 0.90 | 0.19 | 0.00 | 0.65 |
| 22 | 0.00 | 0.00 | 1..46 | 0.05 | 0.78 | 0.26 | 0.07 | 0.46 |
| 23 | 0.00 | 1.71 | 7.90 | 0.26 | 0.79 | 0.21 | 0.05 | 0.51 |
| 24 | 0.00 | 1.55 | 2.91 | 0.47 | 0.86 | 0.19 | 0.02 | 0.62 |
| 25 | 0.08 | 1.02 | 0.88 | 0.70 | 0.89 | 0.17 | 0.02 | 0.67 |
| 26 | 0.14 | 0.97 | 0.09 | 0.96 | 0.96 | 0.16 | 0.01 | 0.75 |
| 27 | 0.00 | 7.65 | 5.73 | 0.60 | 0.96 | 0.80 | 0.02 | 0.08 |
| 28 | 0.00 | 3.26 | 0.00 | 1.00 | 0.96 | 0.08 | 0.00 | 0.80 |
| 30 | 0.86 | 0.00 | 0.00 | 1.00 | 0.94 | 0.22 | 0.01 | 0.67 |
| 29 | 0.00 | 0.00 | 0.18 | 0.85 | 0.96 | 0.78 | 0.02 | 0.11 |

TABLE 8
Overview of reaction conditions for the compositions of
invention examples 31-42 and their respective ratio's.

| | | | | | | | | Gas pressure | |
|---|---|---|---|---|---|---|---|---|---|
| | | | | | catalyst | | | at room | biomass |
| Invention | | Duration** | Temp. | | amount | | | temperature | amount |
| example | Substrate | (min) | (° C.) | Catalyst | (g) | solvent | Gas | (bar) | (g) |
| 31 | pine | 10 | 235 | Ni—Al₂O₃ | 0.20 | MeOH | H₂ | 30 | 2.0 |
| 32 | pine | 30 | 235 | Ni—Al₂O₃ | 0.20 | MeOH | H₂ | 30 | 2.0 |
| 33 | pine | 60 | 235 | Ni—Al₂O₃ | 0.20 | MeOH | H₂ | 30 | 2.0 |
| 34 | pine | 180 | 235 | Ni—Al₂O₃ | 0.20 | MeOH | H₂ | 30 | 2.0 |
| 35 | Birch | 0 | 235 | Ni—Al₂O₃ | 0.20 | MeOH | H₂ | 30 | 2.0 |
| 36 | Birch | 10 | 235 | Ni—Al₂O₃ | 0.20 | MeOH | H₂ | 30 | 2.0 |
| 37 | Birch | 30 | 235 | Ni—Al₂O₃ | 0.20 | MeOH | H₂ | 30 | 2.0 |
| 38 | Birch | 60 | 235 | Ni—Al₂O₃ | 0.20 | MeOH | H₂ | 30 | 2.0 |
| 39 | Birch | 180 | 235 | Ni—Al₂O₃ | 0.20 | MeOH | H₂ | 30 | 2.0 |
| 40 | pine | 180 | 235 | Ni—Al₂O₃ | 0.20 | MeOH | H₂ | 10 | 2.0 |
| 41 | pine | 180 | 235 | Ni—Al₂O₃ | 0.20 | MeOH | H₂ | 2 | 2.0 |
| 42 | pine | 180 | 215 | Ni—Al₂O₃ | 0.20 | MeOH | H₂ | 30 | 2.0 |

| | Biomass/ | oil | | | | | Ratio | Ratio | Ratio | Ratio |
|---|---|---|---|---|---|---|---|---|---|---|
| Invention | catalyst | production/ | % wood | Ratio | Ratio | Ratio | 1 end- | 2 end- | 3 end- | 4 end- |
| example | ratio | wood | retention | β-1 | β-5 | β-β | groups | groups | groups | groups |
| 31 | 10 | 0.04 | 0.83 | 0.75 | 0.26 | 0.40 | 0.83 | 0.71 | 0.09 | 0.05 |
| 32 | 10 | 0.08 | 0.81 | 0.73 | 0.40 | 0.43 | 0.84 | 0.70 | 0.07 | 0.08 |
| 33 | 10 | 0.09 | 0.77 | 0.79 | 0.53 | 0.47 | 0.90 | 0.77 | 0.06 | 0.06 |
| 34 | 10 | 0.12 | 0.73 | 0.87 | 0.71 | 0.56 | 0.93 | 0.70 | 0.04 | 0.15 |
| 35 | 10 | 0.04 | 0.90 | 1.00 | 0.20 | 0.00 | 0.61 | 0.53 | 0.28 | 0.02 |
| 36 | 10 | 0.01 | 0.85 | 0.78 | 0.25 | 0.00 | 0.56 | 0.46 | 0.28 | 0.04 |
| 37 | 10 | 0.09 | 0.89 | 0.64 | 0.32 | 0.00 | 0.69 | 0.58 | 0.23 | 0.05 |
| 38 | 10 | 0.10 | 0.74 | 0.67 | 0.42 | 0.02 | 0.73 | 0.56 | 0.18 | 0.10 |
| 39 | 10 | 0.14 | 0.69 | 0.87 | 0.72 | 0.00 | 0.80 | 0.54 | 0.16 | 0.17 |
| 40 | 10 | 0.10 | 0.77 | 0.27 | 0.32 | 0.51 | 0.64 | 0.38 | 0.06 | 0.14 |
| 41 | 10 | 0.09 | 0.77 | 0.42 | 0.30 | 0.51 | 0.69 | 0.38 | 0.07 | 0.13 |
| 42 | 10 | 0.07 | 0.81 | 0.67 | 0.36 | 0.49 | 0.91 | 0.75 | 0.06 | 0.10 |

*Duration at reaction temperature

TABLE 9
Relative abundance of molecular structures for the compositions
of invention examples 31-42 and their respective ratio's.

| | | | | | | | β-5 | | Ratio | β-β |
|---|---|---|---|---|---|---|---|---|---|---|
| Invention | β-1 | β-1 | β-1 | Ratio | β-5 | β-5 | Phenyl- | β-5 | B-5 | 2x |
| example | y-OH | E | stilbene | β-1 | y-OH | E | coumaran | stilbene | γ-OH | y-OH |
| 31 | 0.38 | 1.00 | 0.46 | 0.75 | 0.45 | 1.00 | 4.09 | 1.04 | 0.26 | 0.62 |
| 32 | 0.49 | 1.00 | 0.56 | 0.73 | 0.37 | 1.00 | 2.02 | 0.70 | 0.40 | 0.68 |
| 33 | 0.47 | 1.00 | 0.38 | 0.79 | 0.43 | 1.00 | 1.26 | 0.61 | 0.53 | 0.73 |
| 34 | 0.33 | 1.00 | 0.20 | 0.87 | 0.32 | 1.00 | 0.53 | 0.43 | 0.71 | 0.95 |
| 35 | 0.00 | 1.00 | 0.00 | 1.00 | 0.00 | 1.00 | 4.05 | 0.00 | 0.20 | 0.00 |
| 36 | 0.47 | 1.00 | 0.41 | 0.78 | 0.00 | 1.00 | 2.96 | 0.00 | 0.25 | 0.00 |
| 37 | 0.00 | 1.00 | 0.57 | 0.64 | 0.00 | 1.00 | 2.16 | 0.00 | 0.32 | 0.00 |
| 38 | 0.00 | 1.00 | 0.50 | 0.67 | 0.00 | 1.00 | 1.38 | 0.74 | 0.42 | 0.02 |
| 39 | 0.37 | 1.00 | 0.21 | 0.87 | 0.21 | 1.00 | 0.46 | 0.82 | 0.72 | 0.00 |
| 40 | 0.00 | 1.00 | 2.66 | 0.27 | 0.05 | 1.00 | 0.79 | 1.49 | 0.32 | 1.00 |
| 41 | 0.40 | 1.00 | 1.90 | 0.42 | 0.14 | 1.00 | 1.04 | 1.63 | 0.30 | 1.00 |
| 42 | 0.27 | 1.00 | 0.62 | 0.67 | 0.20 | 1.00 | 1.14 | 0.97 | 0.36 | 1.00 |

| | | β-β | | | Ratio | Ratio | Ratio | Ratio |
|---|---|---|---|---|---|---|---|---|
| Invention | β-β | c 2x | β-β | Ratio | 1 end- | 2 end- | 3 end- | 4 end- |
| example | THF | y-OH | resinol | β-β | groups | groups | groups | groups |
| 31 | 0.06 | 0.00 | 1.62 | 0.40 | 0.83 | 0.71 | 0.09 | 0.05 |
| 32 | 0.07 | 0.00 | 1.46 | 0.43 | 0.84 | 0.70 | 0.07 | 0.08 |
| 33 | 0.17 | 0.00 | 1.37 | 0.47 | 0.90 | 0.77 | 0.06 | 0.06 |
| 34 | 0.35 | 0.00 | 1.06 | 0.56 | 0.93 | 0.70 | 0.04 | 0.15 |
| 35 | 0.00 | 0.00 | 1.00 | 0.00 | 0.61 | 0.53 | 0.28 | 0.02 |
| 36 | 0.00 | 0.00 | 1.00 | 0.00 | 0.56 | 0.46 | 0.28 | 0.04 |
| 37 | 0.00 | 0.00 | 1.00 | 0.00 | 0.69 | 0.58 | 0.23 | 0.05 |
| 38 | 0.00 | 0.00 | 1.00 | 0.02 | 0.73 | 0.56 | 0.18 | 0.10 |
| 39 | 0.00 | 0.00 | 1.00 | 0.00 | 0.80 | 0.54 | 0.16 | 0.17 |
| 40 | 0.53 | 0.00 | 1.45 | 0.51 | 0.64 | 0.38 | 0.06 | 0.14 |
| 41 | 0.62 | 0.00 | 1.59 | 0.51 | 0.69 | 0.38 | 0.07 | 0.13 |
| 42 | 0.00 | 0.00 | 1.03 | 0.49 | 0.91 | 0.75 | 0.06 | 0.10 |

1.-39. (canceled)

40. Use of an engineered composition comprising aromatic compounds as a starting material or an intermediate composition in the production of a flame retardant,
wherein the molecular mass of the aromatic compounds is between 90 g/mol and 10000 g/mol, wherein the aromatic compounds comprise at least one aromatic compound selected from the formula 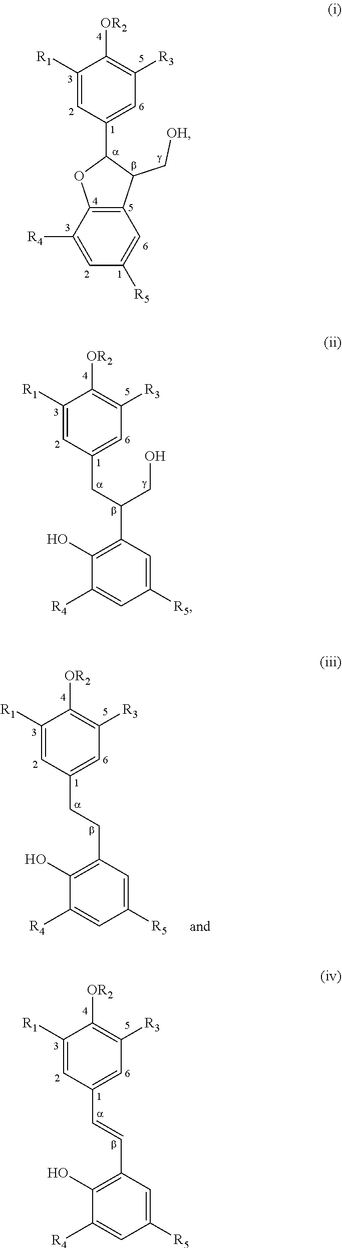
wherein the molecular ratio of ((ii)+(iii))/((i)+(ii)+(iii)+(iv)) is higher than 0.1,
wherein each of R₁, R₃, and R₄ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or any carbon-oxygen linkage to an aromatic monomer or aromatic oligomer and wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO,
—CH═CHCH₂OH, —(CH₂)₂CH₂OCH₃, —CH═CHCH₂OCH₃, —(CH₂)₂CH₂OCH₂CH₃, —CH═CHCH₂OCH₂CH₃,
—(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂,
—(CH₂)₂CH₂O(CH₂)₃CH₃, —CH═CH—CH₂O(CH₂)₃CH₃, or a carbon linkage to an aromatic monomer or aromatic oligomer; and
wherein the aromatic compounds comprise at least one aromatic compound selected from the formulae 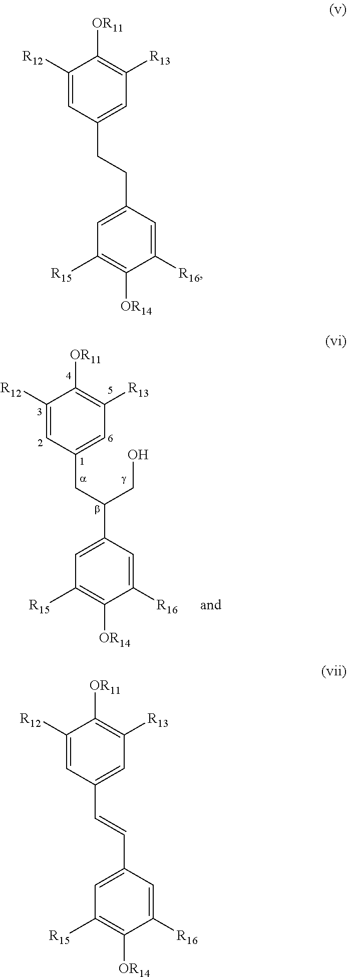
and
wherein the molecular ratio of ((v)+(vi))/((v)+(vi)+(vii)) is higher than 0.15, wherein each of R₁₂, R₁₃, R₁₅ and R₁₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer and
wherein each of R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer; and
wherein the aromatic compounds comprise at least one aromatic compound selected from the formulae 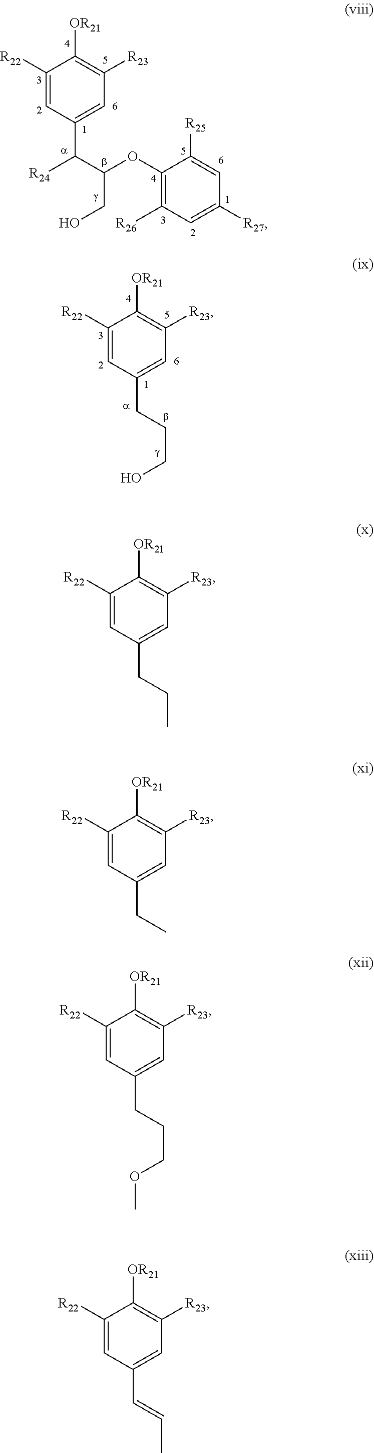 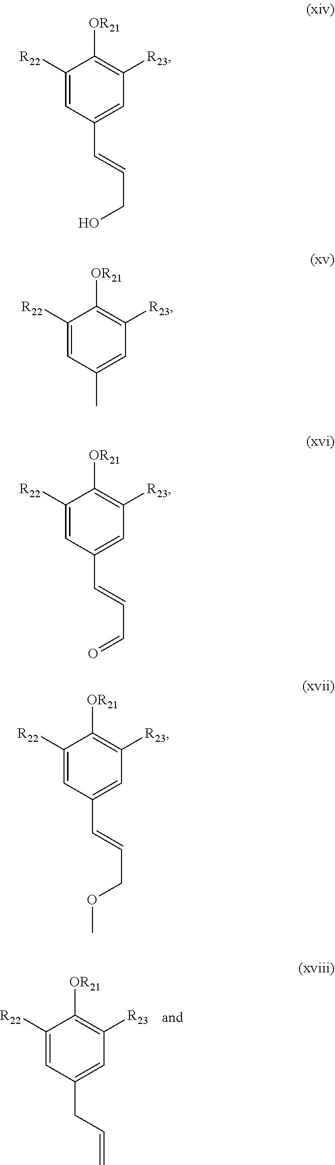
wherein the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.5
wherein each of R₂₂, R₂₃, R₂₅ and R₂₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, a carbon-oxygen linkage to an aromatic monomer or aromatic oligomer, and
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, a carbon-oxygen linkage to an aromatic monomer or aromatic oligomer, and
wherein R₂₄ is independently chosen from —H, —OH, or —O-Alkyl wherein the alkyl group is derived from the alcohol solvent of the process and
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃,
(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃,
—CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂,
—CH═CHCH₂OCH(CH₃)₂, —(CH₂)₂CH₂O(CH₂)₃CH₃, —CH═CHCH₂O(CH₂)₃CH₃ a carbon linkage to an aromatic monomer or an aromatic oligomer.

41. The use according to claim 40, wherein the aromatic compounds comprise at least one aromatic compound selected from the formulae 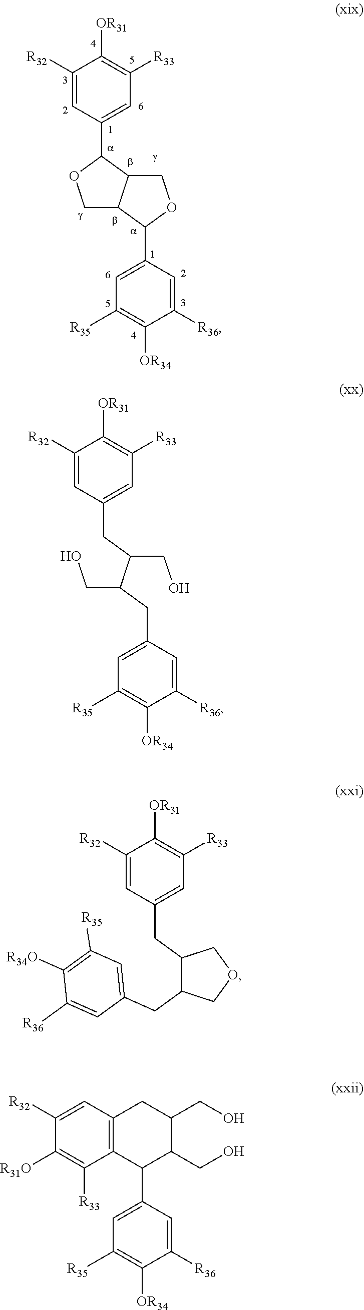
wherein the molecular ratio of ((xx)+(xxi)+(xxii))/((xix)+(xx)+(xxi)+(xxii)) is higher than 0.25, wherein each of R₃₂, R₃₃, R₃₅ and R₃₆ is independently chosen from —H, —OH, —O—CH₃, 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, a carbon-oxygen linkage to an aromatic monomer or aromatic oligomer, and wherein each of R₃₁ and R₃₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer.

42. The use according to claim 40, wherein the composition is composed of aromatic compounds, wherein the molecular mass of the aromatic compounds is between 90 g/mol and 10000 g/mol.

43. The use according to claim 40, wherein the aromatic compounds comprise at least one aromatic compound selected from the formulae 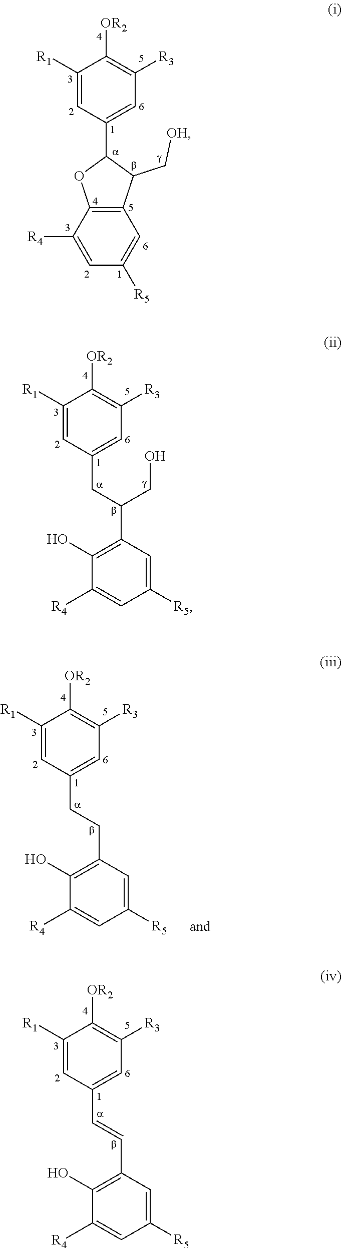
and
wherein the molecular ratio of ((ii)+(iii))/((i)+(ii)+(iii)+(iv)) is higher than 0.7, and preferably higher than 0.9.

44. The use according to claim 40, wherein the aromatic compounds comprise at least one aromatic compound selected from the formulae 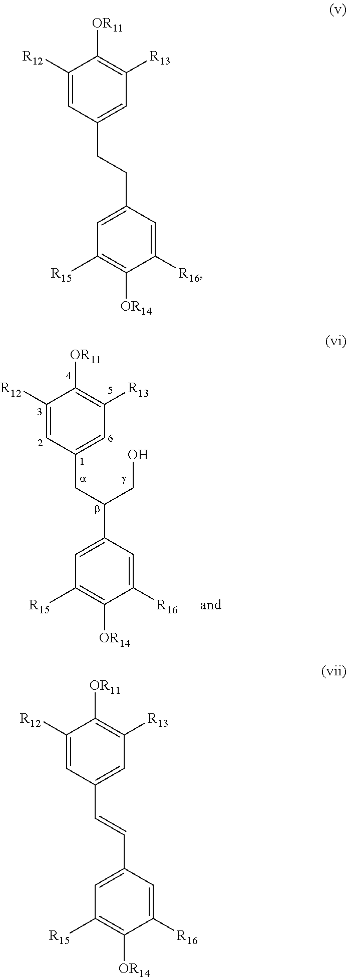
and
wherein the molecular ratio of ((v)+(vi))/((v)+(iv)+(vii)) is higher than 0.6, and preferably higher than 0.9.

45. The use according to claim 40, wherein the aromatic compounds comprise at least one aromatic compound selected from the formulae 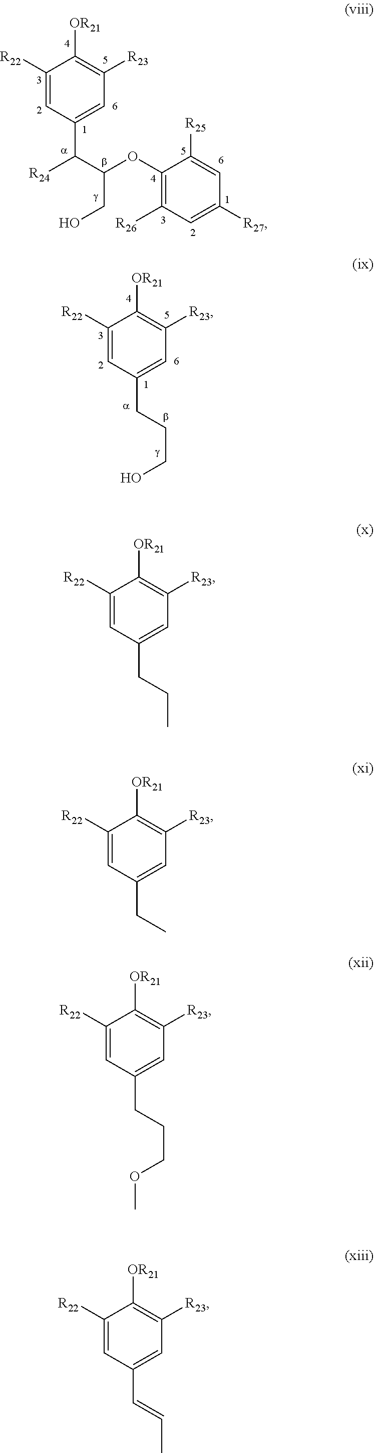 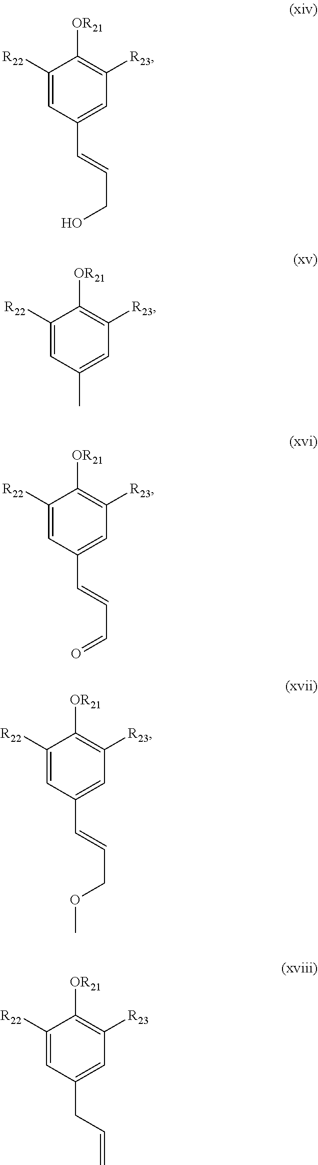
wherein the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.7, and preferably higher than 0.9.

46. The use according to claim 40, wherein the aromatic compounds comprise at least one aromatic compound selected from the formulae 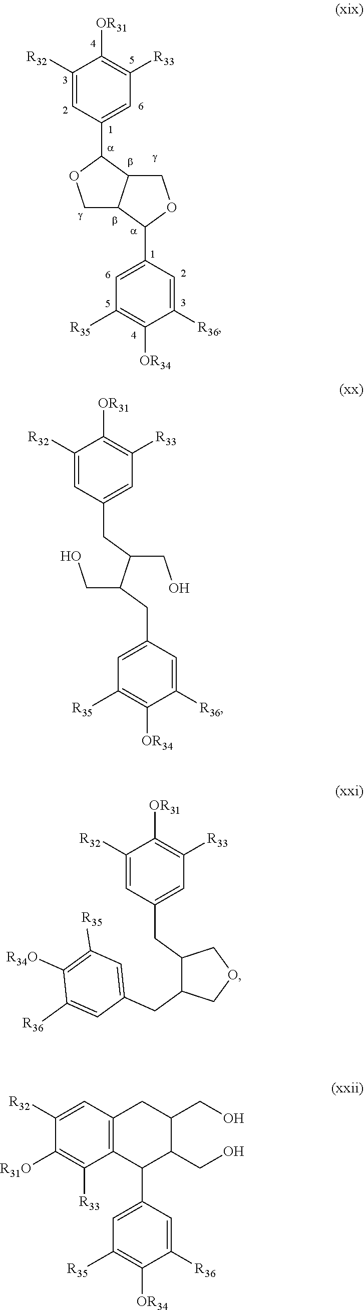
and
wherein the molecular ratio of ((xx)+(xxi)+(xxii))/((xix)+(xx)+(xxi)+(xxii)) is higher than 0.5, and preferably higher than 0.9.

47. The use according to claim 40, wherein the engineered composition has a dispersity index lower than 2.5.

48. The use according to claim 40, wherein the engineered composition has more than 3 mmol aromatic OH per gram of said mixture and more than 1 mmol aliphatic OH per gram of said mixture.

49. The use according to claim 40, wherein the aromatic compounds are phenols and/or phenol ethers.

50. The use according to claim 40, wherein
the molecular ratio of ((ix)]/[(viii)+(ix)+(x)+(xi)+(xii)+(xiii)+(xiv)+(xv)+(xvi)+(xvii)+(xviii)) is higher than 0.5 and the molecular ratio of [(ix)+(x)+(xi)+(xii)]/[(viii)+(ix)+(x)+(xi)+(xii)+(xiii)+(xiv)+(xv)+(xvi)+(xvii)+(xviii)] is higher than 0.7,
more preferably the molecular ratio of ((ix)]/[(viii)+(ix)+(x)+(xi)+(xii)+(xiii)+(xiv)+(xv)+(xvi)+(xvii)+(xviii)) is higher than 0.7 and the molecular ratio of [(ix)+(x)+(xi)+(xii)]/[(viii)+(ix)+(x)+(xi)+(xii)+(xiii)+(xiv)+(xv)+(xvi)+(xvii)+(xviii)] is higher than 0.8,
and most preferably the molecular ratio of ((ix)]/[(viii)+(ix)+(x)+(xi)+(xii)+(xiii)+(xiv)+(xv)+(xvi)+(xvii)+(xviii)) higher than 0.8 and the molecular ratio of [(ix)+(x)+(xi)+(xii)]/[(viii)+(ix)+(x)+(xi)+(xii)+(xiii)+(xiv)+(xv)+(xvi)+(xvii)+(xviii)] is higher than 0.9.

51. The use according to claim 40, wherein the engineered composition is a lignin degradation mixture.

52. The use according to claim 40, wherein the aromatic compounds are lignin derived aromatic compounds.

53. The use according to claim 40, wherein the engineered composition is a lignin conversion in lignin derived aromatic compounds.

54. The use according to claim 40, wherein the engineered composition is a mixture with lignin derived aromatic compounds from catalytic degradation of lignocellulose.

55. The use according to claim 40, wherein the engineered composition is an engineered catalytic degradation product of lignocellulose.

56. The use according to claim 40, wherein the engineered composition is a non-naturally occurring composition.

57. A process for producing an engineered composition comprising aromatic compounds, suitable for use as a starting material or an intermediate composition in the production of a flame retardant,
wherein the molecular mass of the aromatic compounds is between 90 g/mol and 10000 g/mol, wherein the aromatic compounds comprise at least one aromatic compound selected from the formula 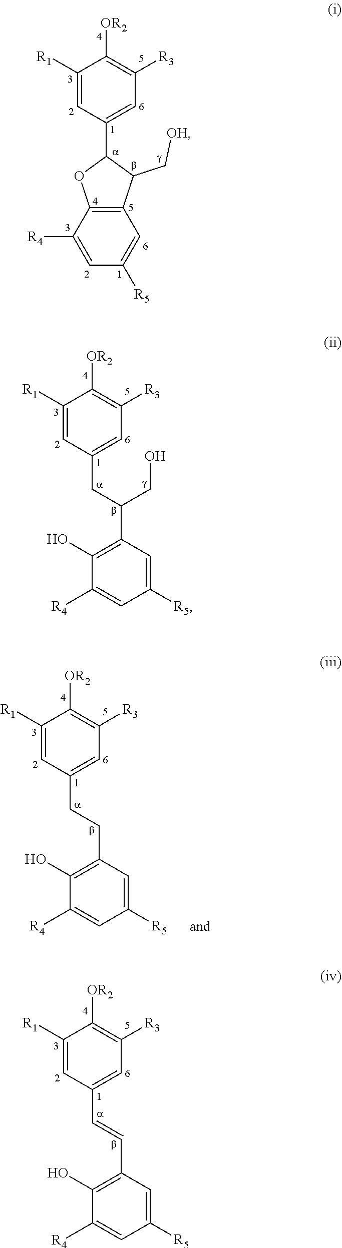
wherein the molecular ratio of ((ii)+(iii))/((i)+(ii)+(iii)+(iv)) is higher than 0.1,
wherein each of R₁, R₃, and R₄ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or any carbon-oxygen linkage to an aromatic monomer or aromatic oligomer and wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an ‘end-unit’ selected from CH₃, —CH₂CH₃, —(CH₂)₂CH₃, —CH₂CH═CH₂, —CH═CHCH₃, —(CH₂)₂CH₂OH, —(CH₂)₂CHO,
—CH═CHCH₂OH, —(CH₂)₂CH₂OCH₃, —CH═CHCH₂OCH₃, —(CH₂)₂CH₂OCH₂CH₃, —CH═CHCH₂OCH₂CH₃,
(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂,
(CH₂)₂CH₂O(CH₂)₃CH₃, —CH═CH—CH₂O(CH₂)₃CH₃, or a carbon linkage to an aromatic monomer or aromatic oligomer; and
wherein the aromatic compounds comprise at least one aromatic compound selected from the formulae 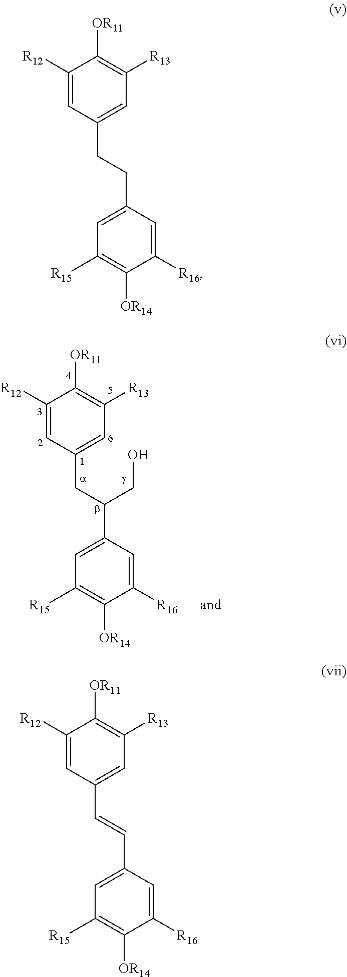
and
wherein the molecular ratio of ((v)+(vi))/((v)+(vi)+(vii)) is higher than 0.15, wherein each of R₁₂, R₁₃, R₁₅ and R₁₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer and wherein each of R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer; and
wherein the aromatic compounds comprise at least one aromatic compound selected from the formulae 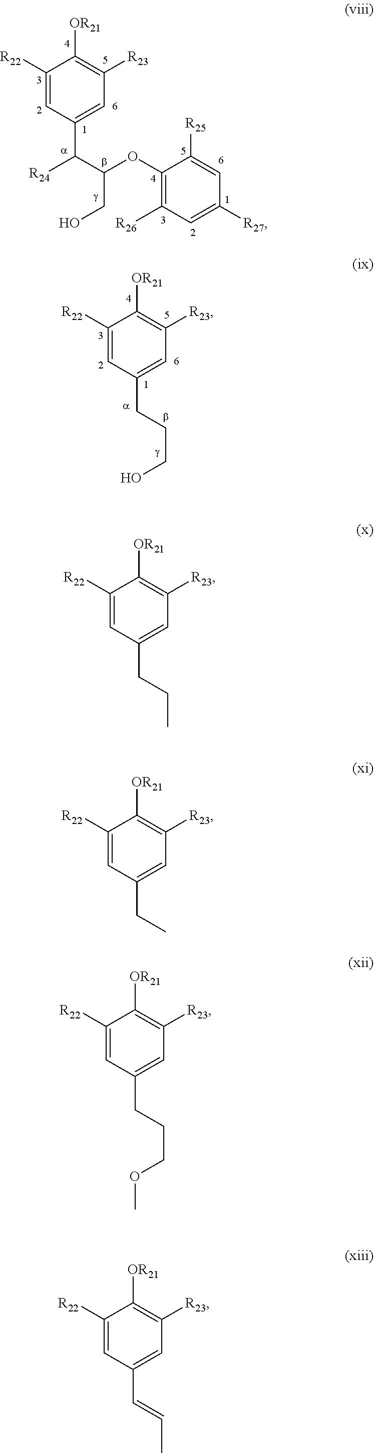 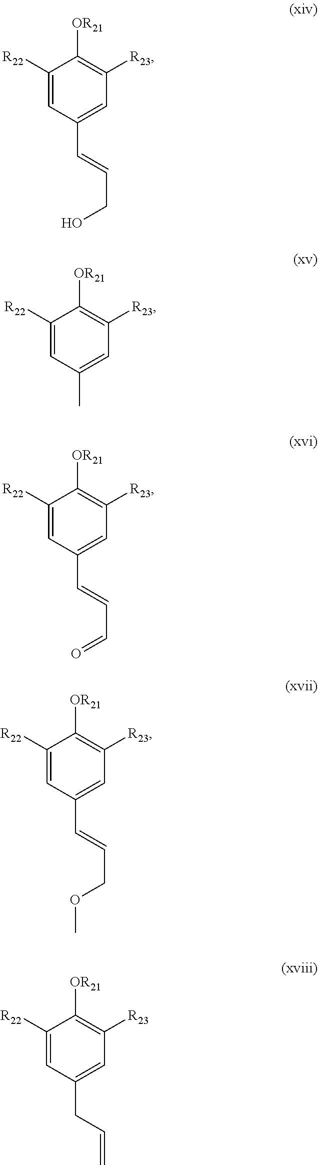
and
wherein the molecular ratio of ((ix)+(x)+(xi)+(xii))/((viii)+(ix)+(x)+(xi)+(xii)+(xiii)+(xiv)+(xv)+(xvi)+(xvii)+(xviii)) in the aromatic mixture is higher than 0.5 wherein each of R₂₂, R₂₃, R₂₅ and R₂₆ is independently chosen from —H, —OH, —O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, a carbon-oxygen linkage to an aromatic monomer or aromatic oligomer, and
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, a carbon-oxygen linkage to an aromatic monomer or aromatic oligomer, and wherein R₂₄ is independently chosen from —H, —OH, or —O-Alkyl wherein the alkyl group is derived from the alcohol solvent of the process and
wherein R₂₇ is independently chosen from —H, a β-O-4 linkage to an aromatic monomer or aromatic oligomer, a β-5 linkage to an aromatic monomer or aromatic oligomer, a β-βlinkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃,
(CH₂)₂CH₂OH, —(CH₂)₂CHO, —CH═CHCH₂OH, —(CH₂)₂CH₂OCH₃, —CH═CHCH₂OCH₃, (CH₂)₂CH₂OCH₂CH₃,
—CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂,
—CH═CHCH₂OCH(CH₃)₂, —(CH₂)₂CH₂O(CH₂)₃CH₃, —CH═CHCH₂O(CH₂)₃CH₃ a carbon linkage to an aromatic monomer or an aromatic oligomer,
The process comprising subjecting a mixture of (A) a feedstock of lignocellulosic material in a feedstock medium comprising an alcohol or alcohol/water mixture and (B) a catalytic medium comprising an alcohol or alcohol/water mixture, hydrogen gas and a catalyst to a temperature of at least 150° C.

58. The process according to claim 57, wherein
(i) lignocellulose, lignocellulosic material or a feedstock comprising lignocellulose in a medium of alcohol or alcohol/water mixture is subjected to a temperature of at least 150° C.;
(ii) a medium comprising a metal catalyst in an alcohol or alcohol/water mixture is subjected to a temperature of at least 150° C. under a hydrogen atmosphere, and
iii) the reaction product of the processed lignocellulosic material is supplied to the catalyst medium.

59. The process according to claim 57, wherein the catalyst comprises nickel in the presence of a hydrogen pressure higher than 10 bar, the reaction time is higher than 0.5 h,
wherein the feedstock is a softwood.
